(19) United States
(12) Patent Application Publication
(10) Pub. No.: US 2024/0384246 A1
(43) Pub. Date: Nov. 21, 2024

(54) CA S9 EFFECTOR PROTEINS WITH ENHANCED STABILITY

(71) Applicant: ASTRAZENECA AB, Södertälje (SE)

(72) Inventors: GRZEGORZ SIENSKI, Södertälje (SE); MARCELLO MARESCA, Södertälje (SE)

(21) Appl. No.: 18/559,598

(22) PCT Filed: May 26, 2022
(86) PCT No.: PCT/EP2022/064368

§ 371 Date: Nov. 8, 2023

Related U.S. Application Data
(60) Provisional application No. 63/193,620, filed on May 27, 2021.

(51) Int. Cl. C12N 9/22; C12N 15/11; C12N 15/90

(52) U.S. Cl. CPC C12N 9/22; C12N 15/11; C12N 15/907; C07K 2319/09; C12N 2310/20

(57) ABSTRACT

Provided are Cas9 effector proteins having enhanced stability. Embodiments of the Cas9 effector proteins have a first nuclear localization signal attached to the N-terminus and a second nuclear localization signal attached to the C-terminus. Also provided are Cas9 systems comprising Cas9 effector proteins having enhanced stability and a guide polynucleotide that forms a complex with the Cas9 effector protein. Further provided are methods for providing site-specific modification of a target sequence in a eukaryotic cell using the Cas9 effector proteins.

### FIELD OF THE INVENTION

The present disclosure provides Cas9 effector proteins having enhanced stability. Embodiments of the Cas9 effector proteins have a first nuclear localization signal attached to the N-terminus and a second nuclear localization signal attached to the C-terminus. The present disclosure also provides Cas9 systems comprising such Cas9 effector proteins and a guide polynucleotide that forms a complex with the Cas9 effector protein. The present disclosure further provides methods for providing site-specific modification of a target sequence in a eukaryotic cell using the Cas9 effector proteins.

### BACKGROUND

The use of the CRIPR/Cas gene editing technology has revolutionized biotechnology. The CRISPR-Cas9 gene editing system has been used successfully in a wide range of organisms and cell lines, both in order to induce double stranded break (DSB) formation in DNA using the wild type Cas9 protein or to nick a single DNA strand using a mutant protein termed Cas9n/Cas9 D10A (see, e.g., Mali et al., Science, 339 (6121): 823-826 (2013) and Sander and Joung, Nature Biotechnology 32(4): 347-355 (2014), each of which is incorporated by reference herein in its entirety). While DSB formation results in creation of small insertions and deletions (indels) that can disrupt gene function, the Cas9n/Cas9 D10A nickase avoids indel creation (the result of repair through non-homologous end-joining) while stimulating the endogenous homologous recombination machinery. Thus, the Cas9n/Cas9 D10A nickase can be used to insert regions of DNA into the genome with high-fidelity.

In addition to genome editing, the CRISPR system has a multitude of other applications, including regulating gene expression, genetic circuit construction, and functional genomics, amongst others (reviewed in Sander and Joung, 2014).

While the Cas9 protein has been shown to be effective in a wide variety of in vivo and in vitro applications, as a protein, it is susceptible to potential degradation, particularly in the cellular environment.

### SUMMARY OF THE INVENTION

The present disclosure is directed to a Cas9 effector protein comprising: a) a first nuclear localization signal attached to the N-terminus of the Cas9 effector protein; and b) a second nuclear localization signal attached to the C-terminus of the Cas9 effector protein.

In some embodiments of the protein, the first nuclear localization signal is a monopartite nuclear localization signal. In some embodiments, the first nuclear localization signal is a bipartite nuclear localization signal. In some embodiments, the second nuclear localization signal is a monopartite nuclear localization signal. In some embodiments, the second nuclear localization signal is a bipartite nuclear localization signal. In some embodiments, the first nuclear localization signal is a bipartite nuclear localization signal and the second nuclear localization signal is a monopartite nuclear localization signal.

In some embodiments, the monopartite nuclear localization signal is SV40 Large T-Antigen, nucleoplasmin, EGL-13, c-Myc, TUS-protein nuclear localization signal, or combinations thereof. In some embodiments, the bipartite nuclear localization signal is classical bipartite nuclear localization signal. In some embodiments, the first nuclear localization signal is classic bipartite nuclear localization signal and the second nuclear localization signal is SV40 Large T-Antigen nuclear localization signal.

In some embodiments of the protein, the first nuclear localization signal is directly attached to the Cas9 effector protein. In some embodiments, the first nuclear localization signal is attached to the Cas9 effector protein via a linker. In some embodiments, the second nuclear localization signal is directly attached to the Cas9 effector protein. In some embodiments, the second nuclear localization signal is attached to the Cas9 effector protein via a linker. In some embodiments, the linker is a peptide linker having from 2 to 30 residues.

In some embodiments, the protein comprises two copies of the first nuclear localization signal. In some embodiments, the protein comprises three copies of the first nuclear localization signal. In some embodiments, the protein comprises two copies of the second nuclear localization signal. In some embodiments, the protein comprises three copies of the second nuclear localization signal.

In some embodiments, the Cas9 effector protein is derived from a bacterial species having a Type II-B CRISPR system. In some embodiments, the Cas9 effector protein comprises a polypeptide sequence having at least 95% identity to any one of SEQ ID NOs: 10-97. In some embodiments, the Cas9 effector protein comprises a domain that matches a TIGR03031 protein family with an E-value cut-off of 1E-5. In some embodiments, the Cas9 effector protein comprises a polypeptide sequence at least 90% identical to SEQ ID NO: 98. In some embodiments, the Cas9 effector protein comprises a modified polypeptide of SEQ ID NO: 98, wherein one or more modifications are selected from N1164R, N1265R, N1300R, N1412R, N347R, N651A, D1266R, D309R, D345R, D487R, D607R, Q1129R, Q1381A, Q1381A, Q1381R, Q661A, Q713R, Q734R, E1032G, E1032R, E1409A, E436R, E611R, E691R, E697R, G1335R, L125R, L1264S, L1299S, K1031R, K490R, K615R, K656R, F636R, S1334A, S1334A, S1334R, S1380R, S1410R, S1413R, S634R, S638R, S711R, S1006R, S1017R, T1267A, T1267R, T551R, Y1338A, Y1338R, V1273S, V1274S, V486R, V644R, V736R and V736Y.

The present disclosure is also directed to a CRISPR-Cas system comprising: a) a Cas9 effector protein comprising: i) a first nuclear localization signal attached to the N-terminus of the Cas9 effector protein; and ii) a second nuclear localization signal attached to the C-terminus of the Cas9 effector protein; and b) a guide polynucleotide comprising a guide sequence and forms a complex with the Cas9 effector protein, wherein the guide sequence is capable of hybridizing with a target sequence in a eukaryotic cell.

The present disclosure is further directed to a CRISPR-Cas system comprising: a) a nucleic acid sequence encoding a Cas9 effector protein comprising: i) a first nuclear localization signal attached to the N-terminus of the Cas9 effector protein; and ii) a second nuclear localization signal attached to the C-terminus of the Cas9 effector protein; and b) a nucleic acid sequence encoding a guide polynucleotide that comprises a guide sequence and forms a complex with the Cas9 effector protein, wherein the guide sequence is capable of hybridizing with a target sequence in a eukaryotic cell.

In some embodiments of the systems, the nucleotide sequences of (a) and (b) are under control of a eukaryotic promoter. In some embodiments, the nucleic acid sequences of (a) and (b) are in a single vector.

The present disclosure is further directed to a CRISPR-Cas system comprising one or more vectors comprising: a) a regulatory element operably linked to one or more nucleotide sequences encoding a Cas9 effector protein comprising: i) a first nuclear localization signal attached to the N-terminus of the Cas9 effector protein; and ii) a second nuclear localization signal attached to the C-terminus of the Cas9 effector protein; and b) a guide polynucleotide that comprises a guide sequence and forms a complex with the Cas9 effector protein, wherein the guide sequence is capable of hybridizing with a target sequence in a eukaryotic cell.

In some embodiments of the systems, the regulatory element is a eukaryotic regulatory element.

In some embodiments of the systems, the first nuclear localization signal is a monopartite nuclear localization signal. In some embodiments, the first nuclear localization signal is a bipartite nuclear localization signal. In some embodiments, the second nuclear localization signal is a monopartite nuclear localization signal. In some embodiments, the second nuclear localization signal is a bipartite nuclear localization signal. In some embodiments, the first nuclear localization signal is a bipartite nuclear localization signal and the second nuclear localization signal is a monopartite nuclear localization signal. In some embodiments, the first nuclear localization signal and the second nuclear localization signal are each bipartite nuclear localization signals.

In some embodiments, the monopartite nuclear localization signal is SV40 Large T-Antigen, nucleoplasmin, EGL-13, c-Myc, TUS-protein nuclear localization signal, or combinations thereof. In some embodiments, the bipartite nuclear localization signal is classical bipartite nuclear localization signal. In some embodiments, the first nuclear localization signal is classic bipartite nuclear localization signal and the second nuclear localization signal is SV40 Large T-Antigen nuclear localization signal.

In some embodiments of the systems, the first nuclear localization signal is directly attached to the Cas9 effector protein. In some embodiments, the first nuclear localization signal is attached to the Cas9 effector protein via a linker. In some embodiments, the second nuclear localization signal is directly attached to the Cas9 effector protein. In some embodiments, the second nuclear localization signal is attached to the Cas9 effector protein via a linker. In some embodiments, the linker is a peptide linker having from 2 to 30 residues.

In some embodiments of the systems, the protein comprises two copies of the first nuclear localization signal. In some embodiments, the protein comprises three copies of the first nuclear localization signal. In some embodiments, the protein comprises two copies of the second nuclear localization signal. In some embodiments, the protein comprises three copies of the second nuclear localization signal.

In some embodiments of the systems, the Cas9 effector protein is derived from a bacterial species having a Type II-B CRISPR system. In some embodiments, the Cas9 effector protein comprises a polypeptide sequence having at least 95% identity to any one of SEQ ID NOs: 10-97. In some embodiments, the Cas9 effector protein comprises a domain that matches a TIGR03031 protein family with an E-value cut-off of 1E-5. In some embodiments, the Cas9 effector protein comprises a polypeptide sequence at least 90% identical to SEQ ID NO: 98.

In some embodiments of the systems, the guide polynucleotide is an RNA. In some embodiments, the guide sequence is from 19 to 30 bases in length. In some embodiments, the guide sequence is from 19 to 25 bases in length. In some embodiments, the guide sequence is from 21 to 26 bases in length. In some embodiments, the guide polynucleotide further comprises a tracrRNA sequence.

In some embodiments of the systems, the Cas9 effector protein generates cohesive ends. In some embodiments, the cohesive ends comprise a single-stranded polynucleotide overhang of 1 to 10 nucleotides. In some embodiments, the cohesive ends comprise a single-stranded polynucleotide overhang of 2 to 6 nucleotides. In some embodiments, the cohesive ends comprise a single-stranded polynucleotide overhang of 3 to 5 nucleotides.

The present disclosure provides a eukaryotic cell comprising a protein as described above. The present disclosure further provides a eukaryotic cell comprising a system as described above.

The present disclosure provides a delivery particle comprising a protein as described above. The present disclosure further provides a delivery particle comprising a system. In some embodiments of the delivery particles, the Cas9 effector protein and the guide polynucleotide are in a complex. In some embodiments, the complex further comprises a polynucleotide comprising a tracrRNA sequence. In some embodiments, the delivery particle further comprises a lipid, a sugar, a metal, or a protein.

The present disclosure provides a vesicle comprising a protein as described above. The present disclosure further provides a vesicle comprising a system as described above. In some embodiments of the vesicles, the Cas9 effector protein and the guide polynucleotide are in a complex. In some embodiments, the vesicle further comprises a polynucleotide comprising a tracrRNA sequence. In some embodiments, the vesicle is an exosome or a liposome.

The present disclosure provides a viral vector comprising a protein as described above. The present disclosure further provides a viral vector comprising a system as described above. In some embodiments, the viral vector further comprises a nucleic acid sequence encoding a tracrRNA sequence. In some embodiments, the viral vector is an adenovirus particle, an adeno-associated virus particle or a herpes simplex virus particle.

The present disclosure also provides a method for providing site-specific modification of a target sequence in a eukaryotic cell, the method comprising:
a) introducing into the cell: i) a nucleotide encoding a Cas9 effector protein comprising: A) a first nuclear localization signal attached to the N-terminus of the Cas9 effector protein; and B) a second nuclear localization signal attached to the C-terminus of the Cas9 effector protein; and ii) a nucleotide encoding a guide polynucleotide that forms a complex with the Cas9 effector protein and comprises a guide sequence, wherein the guide sequence is capable of hybridizing with a target sequence in host polynucleotide;
b) generating cohesive ends in the host polynucleotide with the Cas9 effector protein and the guide polynucleotide; and
c) ligating i) the cohesive ends of (b) together, or ii) a 3′ end of a polynucleotide sequence of interest to one cohesive end, and a 5′ end of the polynucleotide sequence to one cohesive end; thereby modifying the target sequence.

In some embodiments of the methods, the first nuclear localization signal is a monopartite nuclear localization signal. In some embodiments, the first nuclear localization signal is a bipartite nuclear localization signal. In some embodiments, the second nuclear localization signal is a monopartite nuclear localization signal. In some embodiments, the second nuclear localization signal is a bipartite nuclear localization signal. In some embodiments, the first nuclear localization signal is a bipartite nuclear localization signal and the second nuclear localization signal is a monopartite nuclear localization signal. In some embodiments, the monopartite nuclear localization signal is SV40 Large T-Antigen, nucleoplasmin, EGL-13, c-Myc, TUS-protein nuclear localization signal, or combinations thereof. In some embodiments, the bipartite nuclear localization signal is classical bipartite nuclear localization signal. In some embodiments, the first nuclear localization signal and the second nuclear localization signal are each a bipartite nuclear localization signal. In some embodiments, the first nuclear localization signal is classic bipartite nuclear localization signal and the second nuclear localization signal is SV40 Large T-Antigen nuclear localization signal.

In some embodiments of the methods, the first nuclear localization signal is directly attached to the Cas9 effector protein. In some embodiments, the first nuclear localization signal is attached to the Cas9 effector protein via a linker. In some embodiments, the second nuclear localization signal is directly attached to the Cas9 effector protein. In some embodiments, the second nuclear localization signal is attached to the Cas9 effector protein via a linker. In some embodiments, the linker is a peptide linker having from 2 to 30 residues.

In some embodiments of the methods, the protein comprises two copies of the first nuclear localization signal. In some embodiments, the protein comprises three copies of the first nuclear localization signal. In some embodiments, the protein comprises two copies of the second nuclear localization signal. In some embodiments, the protein comprises three copies of the second nuclear localization signal.

In some embodiments of the methods, the Cas9 effector protein is derived from a bacterial species having a Type II-B CRISPR system. In some embodiments, the Cas9 effector protein comprises a polypeptide sequence having at least 95% identity to any one of SEQ ID NOs: 10-97. In some embodiments, the Cas9 effector protein comprises a domain that matches a TIGR03031 protein family with an E-value cut-off of 1E-5.

In some embodiments of the methods, the guide polynucleotide is an RNA. In some embodiments, the guide polynucleotide is from 19 to 30 bases in length. In some embodiments, the guide polynucleotide is from 19 to 25 bases in length. In some embodiments, the guide polynucleotide is from 21 to 26 bases in length. In some embodiments, the guide polynucleotide further comprises a tracrRNA sequence.

In some embodiments of the methods, the Cas9 effector protein generates cohesive ends. In some embodiments, the cohesive ends comprise a single-stranded polynucleotide overhang of 1 to 10 nucleotides. In some embodiments, the cohesive ends comprise a single-stranded polynucleotide overhang of 2 to 6 nucleotides. In some embodiments, the cohesive ends comprise a single-stranded polynucleotide overhang of 3 to 5 nucleotides. In embodiments, the cohesive ends are blunt ends. In embodiments, the cohesive ends have a 5′ single-stranded polynucleotide overhang. In embodiments, the cohesive ends have a 3′ single-stranded polynucleotide overhang.

In some embodiments of the method, the eukaryotic cell is an animal or human cell. In some embodiments, the eukaryotic cell is a human cell. In some embodiments, the eukaryotic cell is a plant cell.

In some embodiments of the method, the modification is deletion of at least part of the target sequence. In some embodiments, the modification is mutation of the target sequence. In some embodiments, the modification is inserting a sequence of interest into the target sequence.

The present disclosure also provides a method for reducing degradation of Cas9 effector protein in a cell comprising a) attaching a first nuclear localization signal to the N-terminus of the Cas9 effector protein; and b) attaching a second nuclear localization signal to the C-terminus of the Cas9 effector protein.

In embodiments of the method, the first nuclear localization signal is a monopartite nuclear localization signal. In embodiments, the first nuclear localization signal is a bipartite nuclear localization signal. In embodiments, the second nuclear localization signal is a monopartite nuclear localization signal. In embodiments, the second nuclear localization signal is a bipartite nuclear localization signal. In embodiments, the first nuclear localization signal is a bipartite nuclear localization signal and the second nuclear localization signal is a monopartite nuclear localization signal. In embodiments, the monopartite nuclear localization signal is SV40 Large T-Antigen, nucleoplasmin, EGL-13, c-Myc, TUS-protein nuclear localization signal, or combinations thereof. In embodiments, the bipartite nuclear localization signal is classical bipartite nuclear localization signal. In embodiments, the first nuclear localization signal is classic bipartite nuclear localization signal and the second nuclear localization signal is SV40 Large T-Antigen nuclear localization signal.

### BRIEF DESCRIPTION OF THE DRAWINGS

The following drawings form part of the present specification and are included to further demonstrate exemplary embodiments of certain aspects of the present invention.

FIG. 1 provides amino acid sequences of Cas9 proteins that can be used in the Cas9 effector proteins described herein.

FIG. 2 provides additional amino acid sequences of Cas9 proteins that can be used in the Cas9 effector proteins described herein.

FIG. 3A is a western blot showing expression of MHCas9 in the absence or presence of the inhibitors: 1) proteasome inhibitor MG132 at a concentration of 5 μM; 2) lysosomal vATPase inhibitor bafilomycin A1 at a concentration of 20 nM; or 3) nuclear export inhibitor leptomycin B at a concentration of 10 nM as described in Example 1. FIG. 3B is a bar graph of quantification of the western blot using MAPK for normalization for each inhibitor and control.

FIG. 4 is a western blot showing expression of the Cas9 constructs: 1) 3×SV40-MHCas9; 2) MHCas9-NLSSV40; 3) 3XNLSSV40-MHCas9-NLSSV40; and 4) bpNLS-MHCas9-SLSSV40 (SpOT-ON) as described in Example 2. GFP expressed by the cloning vector is detected as a transfection control, while tubulin is detected as a gel loading control.

FIG. 5 shows western blots of the expression of SpOT-ON (5A) or bpNLS-SpCas9-NLSSV40 (5B) as described in Example 3. The Cas9 constructs were tested in the absence or presence of the inhibitors: 1) proteasome inhibitor MG132 at a concentration of 5 μM; 2) lysosomal vATPase inhibitor bafilomycin A1 at a concentration of 20 nM; or 3) nuclear export inhibitor leptomycin B at a concentration of 10 nM.

FIG. 6 shows plots of titrations of DNA cleavage activity at different sites with either SpOT-ON (MHCas9) or SpCas9 as described in Example 4.

FIG. 7 shows a bar graph of the DNA cleavage speed constant k when different protospacer lengths are used as described in Example 5.

FIG. 8 shows bar graphs plotting show the mean percentage of mutated reads in mapped reads for different protospacer lengths at the EMX1 site (8A) and the CD34 site (8B) as described in Example 6. Bar graphs represent average editing efficiency of HEK293T-cells±SD of n=3 different PBMC donors targeting CD34 or EMX1 as assessed through Amplicon-Seq and RIMA analysis. Allele frequency <0.1% were excluded from the analysis.

FIG. 9 shows a plot of the percentage of modified reads at off-target sites for SpOT-ON and SPCas9 as described in Example 7.

FIG. 10 shows a bar graph plotting cleavage speed constants for DNA substrates having mismatches at positions 1, 2 and 3 from the PAM as described in Example 8.

FIG. 11 shows a bar graphs of the mean percentage of mutated reads in mapped reads at different positions for mismatch editing of EMX1 was tested with a 23 nucleotide guide RNA as described in Example 9. Bar graphs represent average editing efficiency of HEK293T-cells±SD of n=3 different PBMC donors targeting EMX1 as assessed through Amplicon-Seq and RIMA analysis. Allele frequency <0.1% were excluded from the analysis.

FIG. 12 shows qualitative analysis of DNA editing at the EMX locus (FIG. 12A) and CD34 locus (FIG. 12B) as described in Example 10. FIG. 12C shows qualitative analysis of the comparison of DNA repair after SpCas9 DNA cleavage at the CD34 locus.

FIG. 13 is a bar graph showing the percentage of non-homologous end joining (NHEJ) knock-in at the CD34 locus for substrates having different overhangs as shown.

Experiments were performed as in Example 11. Plots are shown for both potential directionalities of the insert, with dark grey representing forward (expected) insertion and light grey representing reverse insertion.

FIG. 14 is a bar graph showing the percentage of NHEJ knock-in at the STAT1 locus for substrates having different overhangs as shown. Experiments were performed as in Example 11. Plots are shown for both potential directionalities of the insert, with dark grey representing forward (expected) insertion and light grey representing reverse insertion.

### DETAILED DESCRIPTION OF THE INVENTION

The present disclosure provides Cas9 effector proteins having enhanced stability which comprise a nuclear localization signal on both the N-terminus and the C-terminus of the Cas9 effector protein. The present disclosure also provides systems comprising a Cas9 effector protein having enhanced stability and a nucleic acid guide sequence that complexes with the Cas9 effector protein. The present disclosure also provides methods for site-specific modification of a target sequence in a eukaryotic cell using a Cas9 effector protein having enhanced stability. The present disclosure further provides methods for enhancing the stability of Cas9 effector proteins by attaching nuclear localization signals to both the N-terminus and the C-terminus of the protein.

Without wishing to be bound by theory, it is thought that the presence of additional nuclear localization signals on the Cas9 effector protein lead to enhanced nuclear import of the protein. This enhanced nuclear import causes the Cas9 effector protein to spend less time in the cytoplasm, where it can be a substrate for lysosomal degradation, which is a common breakdown pathway for cytosolic proteins. In embodiments, the Cas9 effector proteins described herein have enhanced stability but retain significant Cas9 effector activity compared to Cas9 proteins not having enhanced stability.

As used herein, a protein having “enhanced stability” means a protein with a longer life in an in vivo environment, e.g., a cell, or inside of an in vitro environment. In some embodiments, a protein having “enhanced stability” can be more resistant to degradation in the environment, by having less exposure to factors that degrade proteins, such as proteases, and/or by being a poorer substrate to a factor that degrades proteins, e.g., by being more resistant to the cleavage of bonds within the protein. In embodiments, the “enhanced stability” is enhanced compared to a protein in an unmodified state. In embodiments, the “enhanced stability” of a Cas9 effector protein as described herein is enhanced compared to a Cas9 effector protein that does not have a nuclear localization signal. In embodiments, the “enhanced stability” of a Cas9 effector protein as described herein is enhanced compared to a Cas9 effector protein that only has one nuclear localization signal. In embodiments, the “enhanced stability” of a Cas9 effector protein as described herein is enhanced compared to a Cas9 effector protein that only has one nuclear localization signal that is attached the N-terminus of the Cas9 effector protein. In some embodiments, the stability of the Cas9 effector protein is enhanced greater than 10%, greater than 20%, greater than 30%, greater than 40%, greater than 50%, greater than 60%, greater than 70%, greater than 80%, greater than 90%, greater than 100%, greater than 120%, greater than 140%, greater than 160%, greater than 180%, greater than 200%, greater than 300%, or greater than 400% after 30 minutes of expression, 60 minutes of expression, 90 minutes of expression, 120 minutes of expression, 150 minutes of expression, 120 minutes of expression, as measured by means known to the skill artisan for determining the quantity of a protein (e.g., Western blot) or by means known to the skilled artisan for determining the quantity of a protein by measuring the activity of the protein (e.g., the activity assays described herein).

As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability, depending on the situation. In embodiments, one of skill in the art will understand the level of variability indicated by the term “about,” due to the context in which it is used herein. It should also be understood that use of the term “about” also includes the specifically recited value.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

### Cas9 Effector Proteins

In embodiments, the present disclosure provides a Cas9 effector protein having enhanced stability. In embodiments, the present disclosure provides a Cas9 effector protein comprising more than one nuclear localization signal. In embodiments, the present disclosure provides a Cas9 effector protein comprising: a) a first nuclear localization signal attached to the N-terminus of the Cas9 effector protein; and b) a second nuclear localization signal attached to the C-terminus of the Cas9 effector protein.

As described herein, Cas proteins are components of the CRISPR-Cas system, which can be used for, inter alia, genome editing, gene regulation, genetic circuit construction, and functional genomics. While the Cas1 and Cas2 proteins appear to be universal to all the presently identified CRISPR systems, the Cas3, Cas9, and Cas10 proteins are thought to be specific to the Type I, Type II, and Type III CRISPR systems, respectively.

Following initial publications around the CRISPR-Cas9 system (Type II system), Cas9 variants have been identified in a range of bacterial species and a number have been functionally characterized. See, e.g., Chylinski et al., “Classification and evolution of type II CRISPR-Cas systems”, Nucleic Acids Research 42(10): 6091-6105 (2014), Ran et al., “In vivo genome editing using Staphylococcus aureus Cas9”, Nature 520(7546): 186-91 (2015), and Esvelt et al., “Orthogonal Cas9 proteins for RNA-guided gene regulation and editing”, Nature Methods 10(11): 1116-1121 (2013), each of which is incorporated by reference herein in its entirety.

The present disclosure encompasses novel effector proteins of CRISPR-Cas9 systems having enhanced Cas9 stability. The terms “Cas9,” “Cas 9 protein” and “Cas9 effector protein” are interchangeable and are used herein to describe effector proteins which are capable of providing cohesive ends, blunt end or nicked dsDNA when used in the CRISPR-Cas9 system.

In embodiments, the nuclear localization signals are monopartite nuclear localization signals, bipartite nuclear localization signals or combinations thereof. A nuclear localization signal, also called a nuclear localization sequence or NLS, is an amino acid sequence that causes a protein having the sequence to be imported into the cell nucleus. In embodiments, a monopartite nuclear localization signal is a signal having a single contiguous sequence that is recognized for nuclear import. In embodiments, a bipartite nuclear localization signal is a signal having two sequences that are recognized for nuclear import separated by a spacer sequence. Examples of both monopartite and bipartite nuclear localization signals are provided herein.

In embodiments of the Cas9 effector protein, the first nuclear localization signal is a monopartite nuclear localization signal. In embodiments, the first nuclear localization signal is a bipartite nuclear localization signal. In embodiments, the second nuclear localization signal is a monopartite nuclear localization signal. In embodiments, the second nuclear localization signal is a bipartite nuclear localization signal.

In embodiments, the first and second nuclear localization signals can both be monopartite, both be bipartite or can be a mixture of monopartite and bipartite. In embodiments, the first nuclear localization signal is a bipartite nuclear localization signal and the second nuclear localization signal is a monopartite nuclear localization signal. In embodiments, the first nuclear localization signal is a monopartite nuclear localization signal and the second nuclear localization signal is a bipartite nuclear localization signal. In embodiments, the first nuclear localization signal is a monopartite nuclear localization signal and the second nuclear localization signal is a monopartite nuclear localization signal. In embodiments, the first nuclear localization signal is a bipartite nuclear localization signal and the second nuclear localization signal is a bipartite nuclear localization signal.

In embodiments, the monopartite nuclear localization signal is a monopartite nuclear localization signal known in the art. In embodiments, the monopartite nuclear localization signal is one of the monopartite nuclear localization signals listed in Table 1, or combinations thereof.

TABLE 1
Monopartite Nuclear Localization Signals

| | | SEQ |
|---|---|---|
| | | ID |
| NLS Name | Sequence | NO: |
| SV40 Large T-Antigen | PKKKRKV | 1 |
| SV40 Large T-Antigen | PKKKRRV | 2 |
| Nucleoplasmin | AVKRPAATKKAGQAKKKKLD | 3 |
| EGL-13 | MSRRRKANPTKLSENAKKLAKEVEN | 4 |
| c-Myc | PAAKRVKLD | 5 |
| TUS-protein | KLKIKRPVK | 6 |

In embodiments, the bipartite nuclear localization signal is a bipartite nuclear localization signal known in the art. In embodiments, the bipartite nuclear localization signal is a classical bipartite nuclear localization signal. In embodiments, the bipartite nuclear localization signal is one of the bipartite nuclear localization signals listed in Table 2, or combinations thereof.

TABLE 2
Bipartite Nuclear Localization Signals

| | | SEQ |
|---|---|---|
| | | ID |
| NLS Name | Sequence | NO: |
| Classical Bipartite | KRTADGSEFESPKKKRKV | 7 |
| Classical Bipartite | KRPAATKKAGQAKKKK | 8 |
| Classical Bipartite | KRPAATKKAGQAKKK | 9 |

In embodiments of the protein, the first nuclear localization signal is classic bipartite nuclear localization signal (SEQ ID NO: 7) and the second nuclear localization signal is SV40 Large T-Antigen nuclear localization signal (SEQ ID NO: 1).

In embodiments, the nuclear localization signals are attached to the Cas9 effector protein using standard methods in the art. In embodiments, nucleic acid sequences encoding for the nuclear localization signals are placed upstream and downstream from a nucleic acid sequence encoding the Cas9 effector protein using standard molecular biology methods such as restriction enzyme digestion and ligation, so that a nucleic acid is formed that encodes the Cas9 effector protein comprising a nuclear localization signal on its N-terminus and C-terminus. This nucleic acid can then be subsequently expressed in a cell, e.g., a eukaryotic cell. In other embodiments, the Cas9 effector protein comprising nuclear localization signals on its N-terminus and C-terminus is fully or partially synthesized using solid-phase protein synthesis methods.

In embodiments of the protein, the first nuclear localization signal is directly attached to the Cas9 effector protein. In embodiments, the first nuclear localization signal is attached to the Cas9 effector protein via a linker. In embodiments, the second nuclear localization signal is directly attached to the Cas9 effector protein. In embodiments, the second nuclear localization signal is attached to the Cas9 effector protein via a linker.

In embodiments where a linker is used, the linker is a peptide linker having from 2 to 30 residues. In embodiments, linker is a peptide linker having from 2 to 20 residues. In embodiments, linker is a peptide linker having from 2 to 15 residues. In embodiments, linker is a peptide linker having from 2 to 10 residues. In embodiments, linker is a peptide linker having from 2 to 5 residues. In embodiments, the linker is a substituted or unsubstituted C₂-C₂₀ alkyl, alkene or alkynyl chain.

In embodiments, the Cas9 effector protein comprises more than one copy of a nuclear localization signal on its N-terminus. In embodiments, the Cas9 effector protein comprises more than one type of nuclear localization signal on its N-terminus. In embodiments, the Cas9 effector protein comprises more than one copy of a nuclear localization signal on its C-terminus. In embodiments, the Cas9 effector protein comprises more than one type of nuclear localization signal on its C-terminus.

In embodiments, the protein comprises two copies of the first nuclear localization signal. In embodiments, the protein comprises three copies of the first nuclear localization signal. In embodiments, the protein comprises two copies of the second nuclear localization signal. In embodiments, the protein comprises three copies of the second nuclear localization signal.

The Cas9 portion of the Cas9 protein comprising a first and a second nuclear localization signal can be derived from any Cas9 effector domain known in the art. In embodiments, the Cas9 effector protein is derived from a bacterial species having a Type II-B CRISPR system. Examples of suitable Type II-B Cas9 proteins are described in WO/2019/099943, which is hereby incorporated by reference herein. In embodiments, suitable Type II-B Cas9 are capable of generating cohesive ends. As described herein, Type II-B CRISPR systems are identified, inter alia, by the presence of a cas4 gene on the cas operon, and Type II-B Cas9 proteins is of the TIGR03031 TIGRFAM protein family. Thus, in embodiments, the Cas9 portion is of the TIGR03031 TIGRFAM protein family. In embodiments, the Cas9 portion comprises a domain that matches the TIGR03031 protein family with an E-value cut-off of 1E-5. In embodiments, the site-specific nuclease comprises a domain that matches the TIGR03031 protein family with an E-value cut-off of 1E-10. Type II-B CRISPR systems are found in bacterial species such as, e.g., Legionella pneumophila, Francisella novicida, gamma proteobacterium HTCC5015, Parasutterella excrementihominis, Sutterella wadsworthensis, Sulfurospirillum sp. SCADC, Ruminobacter sp. RM87, Burkholderiales bacterium 1_1_47, Bacteroidetes oral taxon 274 str. F0058, Wolinella succinogenes, Burkholderiales bacterium YL45, Ruminobacter amylophilus, Campylobacter sp. P0111, Campylobacter sp. RM9261, Campylobacter lanienae strain RM8001, Camplylobacter lanienae strain P0121, Turicimonas muris, Legionella londiniensis, Salinivibrio sharmensis, Leptospira sp. isolate FW.030, Moritella sp. isolate NORP46, Endozoicomonas sp. S-B4-1U, Tamilnaduibacter salinus, Vibrio natriegens, Arcobacter skirrowii, Francisella philomiragia, Francisella hispaniensis, or Parendozoicomonas haliclonae.

In some embodiments, the Cas9 is capable of generating a double-stranded polynucleotide cleavage, e.g., a double-stranded DNA cleavage. In some embodiments, a Cas9 can include one or more nuclease domains, such as RuvC and HNH, and can cleave double-stranded DNA. In some embodiments, a Cas9 can comprises a RuvC domain and an HNH domain, each of which cleaves one strand of double-stranded DNA. In some embodiments, the Cas9 generates blunt ends. In some embodiments, the RuvC and HNH of a Cas nuclease cleaves each DNA strand at the same position, thereby generating blunt ends. In some embodiments, the Cas9 generates cohesive ends. In some embodiments, the RuvC and HNH of a Cas9 cleaves each DNA strand at different positions (i.e., cut at an “offset”), thereby generating cohesive ends. As used herein, the terms “cohesive ends,” “staggered ends,” or “sticky ends” refer to a nucleic acid fragment with strands of unequal length. In contrast to “blunt ends,” cohesive ends are produced by a staggered cut on a double-stranded nucleic acid (e.g., DNA). A sticky or cohesive end has protruding singles strands with unpaired nucleotides, or “overhangs,” e.g., a 3′ or a 5′ overhang.

In embodiments, the term Cas9 refers to engineered Cas9 variants, such as, e.g., deadCas9-FokI, Cas9n^{D10A}-FokI, and Cas9n^{H840A}-FokI. In embodiments of the disclosure, the Cas9 effector proteins comprise: a) a first nuclear localization signal attached to the N-terminus of the Cas9 effector protein; and b) a second nuclear localization signal attached to the C-terminus of the Cas9 effector protein.

In some embodiments, the Cas9 (e.g., the Cas9 domain of the fusion protein) comprises a nuclease-inactivated Cas9 (e.g., a Cas9 lacking DNA cleavage activity; “dCas9”) that retains RNA (gRNA) binding activity and is thus able to bind a target site complementary to a gRNA. In embodiments, the fusion protein comprises a linker between the dCas9 domain and the transcriptional regulator domain. In embodiments, the dCas9 domain is fused with a transcriptional activator or repressor domain, forming a dCas9 transcriptional regulator that can be directed to a specific target site through a complementary gRNA sequence. Examples of linkers are described herein. In embodiments, the fusion protein of a dCas9 domain and transcriptional regulator domain has a nuclear localization signal, as described herein, attached to the N-terminus of the dCas9 domain and nuclear localization signal attached to the C-terminus of the transcriptional regulator domain.

In embodiments, the dCas9 domain is a dCas9 domain that functions as a roadblock blocking transcription. In embodiments, the dCas9 domain can sterically block the transcriptional elongation of RNA polymerase.

In embodiments, the dCa9 domain is fused to a VP64 transcriptional activation domain. In embodiments, the dCas9 domain is modified using the SunTag gene activating system where tandem repeats of a small peptide GCN4 are utilized to recruit multiple copies of single-chain variable fragments in fusion with the transcriptional activator VP64. In embodiments, the dCas9 domain is modified using the synergistic activation mediator (SAM) system where the dCas9 is fused to VP64 and the sgRNA has been modified to contain two MS2 RNA aptamers to recruit the MS2 bacteriophage coat protein (MCP), which is fused to the transcriptional activators p65 and heat shock factor 1 (HSF1). In embodiments, the dCas9 domain is modified with VP64-p65-Rta (VPR) for gene activation, where the dCas9 is fused to the combinatory VPR transcriptional activator domains to amplify the activation effects. In embodiments, the dCas9 domain is modified with scRNA for simultaneous gene activation and repression, where a hybrid RNA scaffold coupling an sgRNA and an RNA aptamer (e.g., MS2, com, PP7) can recruit RNA-binding proteins s (e.g., MCP, COM, PCP) tethered to either a transcriptional activator or repressor.

In embodiments, the dCas9 domain is modified with a chemical or light controlled dimerization systems, where chemical or light induced dimerizers (e.g., PYL1::ABI, GID::GAI and PhyB::PIF) are fused to dCas9 and transcriptional effectors, respectively.

In these embodiments, the addition of corresponding chemical (e.g., abscisic acid [ABA], or gibberellin [GA]) or light can induce the gene regulation. In embodiments, the dCas9 domain is modified using a split dCas system or a receptor-coupled systems: I/O molecular devices.

In embodiments, the dCas9 is a second generation or third generation transcriptional regulator as described in Xu et al., “A CRISPR-dCas Toolbox for Genetic Engineering and Synthetic Biology,” J. Mol. Biol., 2019, 431:34-47, which is hereby incorporated by reference herein.

In embodiments, the dCas9 is a dCas9 fusion protein for epigenome engineering. In embodiments, the dCas9 for epigenome engineering is a dCas9 fusion protein as described in Xu et al., J. Mol. Biol., 2019, 431:34-47, which is hereby incorporated by reference herein. In embodiments, the dCas9 is fused to a methyltransferase, e.g., DNMT3A, DNMT3B or DNMT3L. In embodiments, the dCas9 is fused to a KRAB domain. In embodiments, the dCas9 is fused to a DNA demethylase, e.g. TET1. In embodiments, the dCas9 is fused to a histone methyltransferase, e.g., PRDM9 or DOT1L. In embodiments, the dCas9 is fused to a histone demethylase, e.g. LSD1. In embodiments, the dCas9 is fused to a histone acetyltransferase, e.g., p300. In embodiments, the dCas9 is fused to a histone deacetylase, e.g., HDAC1, HDAC2, HDAC3, HDAC4, HDAC5, HDAC6, HDAC7, HDAC8, HDAC9, HDAC 10 or HDAC11, or SIRT1, SIRT2, SIRT3, SIRT4, SIRT5, SIRT6 or SIRT7.

In embodiments, the dCas9 is a dCas9 fusion protein for genome imaging. In embodiments, the dCas9 for genome imaging is a dCas9 fusion protein as described in Xu et al., J. Mol. Biol., 2019, 431:34-47, which is hereby incorporated by reference herein. In embodiments, the dCas9 is fused to a fluorescent protein, e.g., a green fluorescent protein, a yellow fluorescent protein, a blue fluorescent protein, a cyan fluorescent protein, an orange fluorescent protein or a red fluorescent protein.

In embodiments, the dCas9 is a dCas9 fusion protein for base editing. In embodiments, the dCas9 is fused to a cytosine base editor. In embodiments, the dCas9 is fused to an adenine base editor. In embodiments, the dCas9 is fused to a uracil base editor. In embodiments, the dCas9 is fused to a cytidine deaminase. In embodiments, the dCas9 is fused to an adenine deaminase. In embodiments, the dCas9 is fused to a uracil DNA glycosylase.

In embodiments, the Cas9 domain is a Cas9 nickase fusion protein for base editing. A “Cas9 nickase” as used herein is a Cas9 protein that only cleaves one strand of the target DNA. In embodiments, the Cas9 nickase is fused to a cytosine base editor. In embodiments, the Cas9 nickase is fused to an adenine base editor. In embodiments, the Cas9 nickase is fused to a uracil base editor. In embodiments, the Cas9 nickase is fused to a cytidine deaminase. In embodiments, the Cas9 nickase is fused to an adenine deaminase. In embodiments, the Cas9 nickase is fused to a uracil DNA glycosylase. In embodiments, the Cas9 domain is a Cas9 nickase fusion for base editing as described in US2018/0312828, US2018/0237787 and US2020/0010835, each of which is hereby incorporated by reference herein.

In embodiments, the Cas9 domain is a Cas9 nickase fusion protein for prime editing. In embodiments, the Cas9 nickase is fused to a reverse transcriptase. In embodiments the Cas9 domain is a Cas9 nickase fusion for prime editing as described in WO2020/191248, which is hereby incorporated by reference herein.

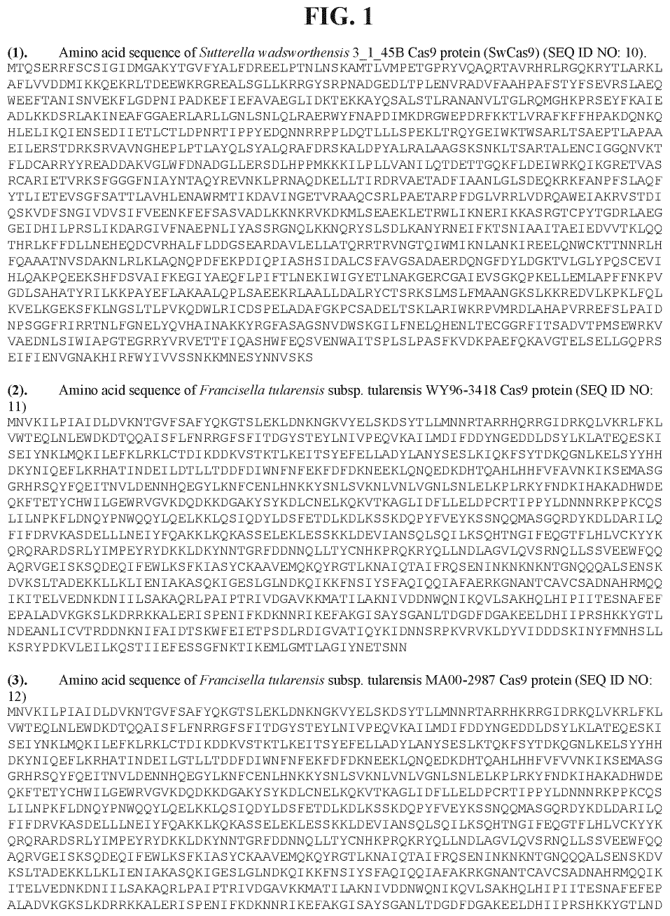

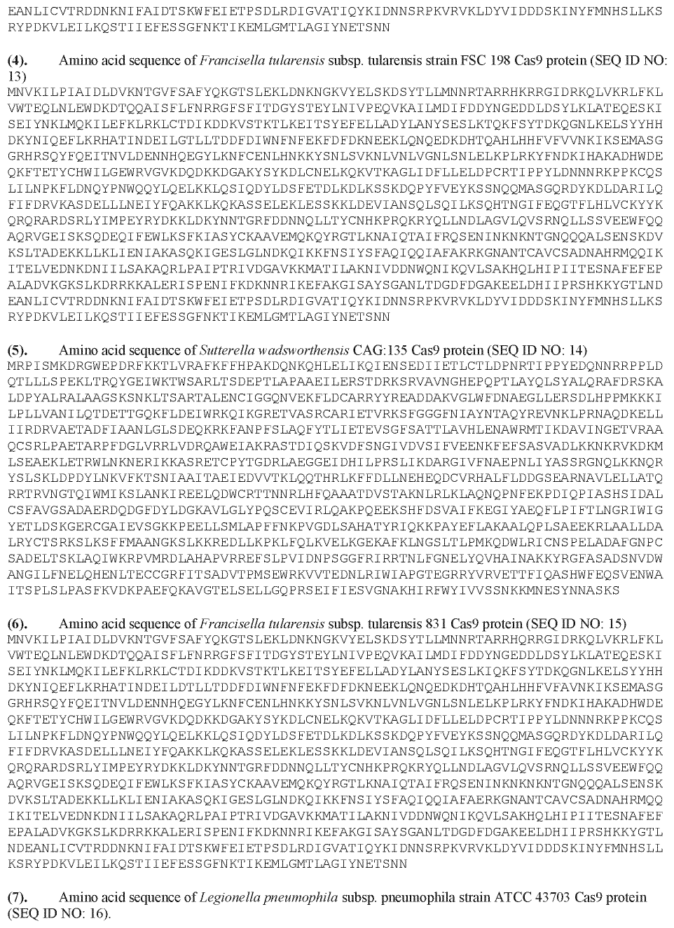

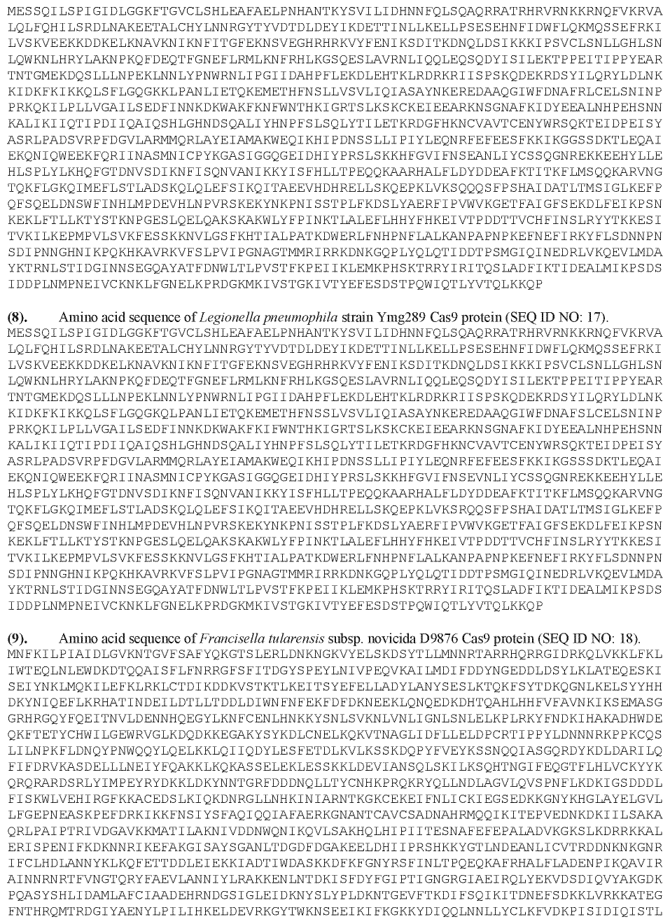

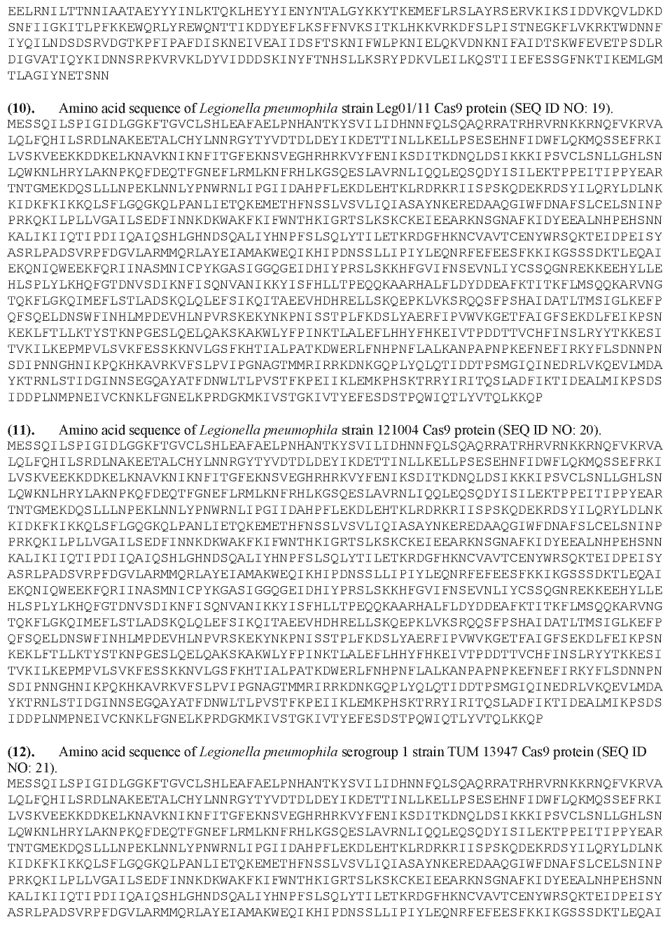

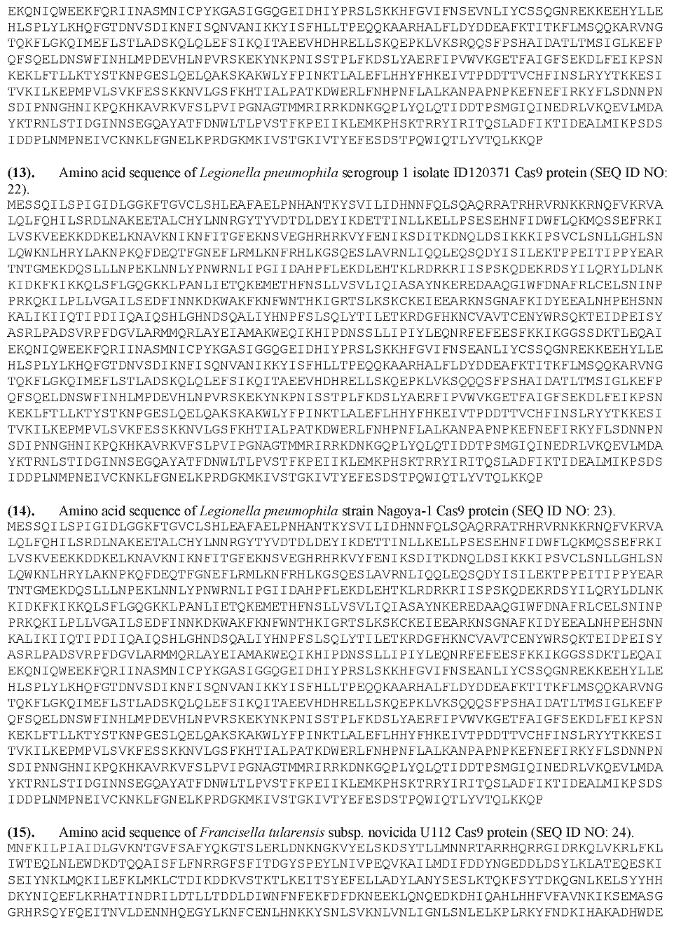

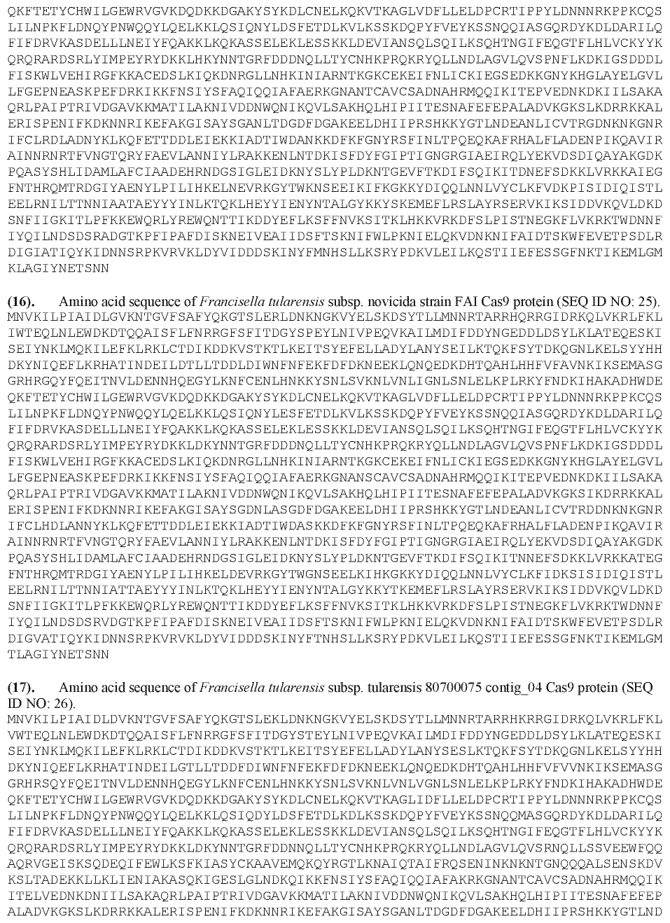

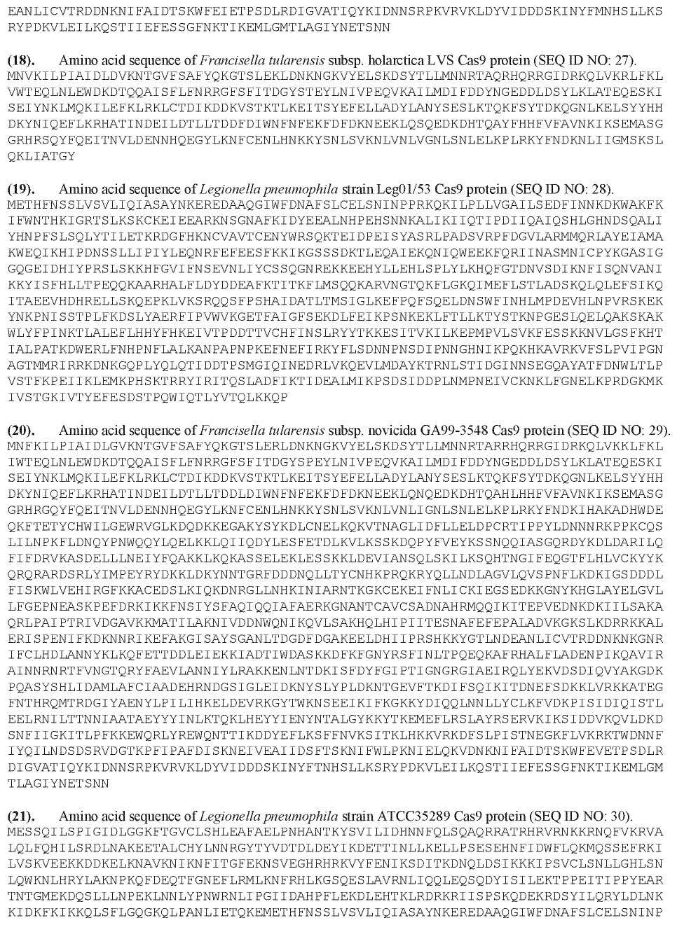

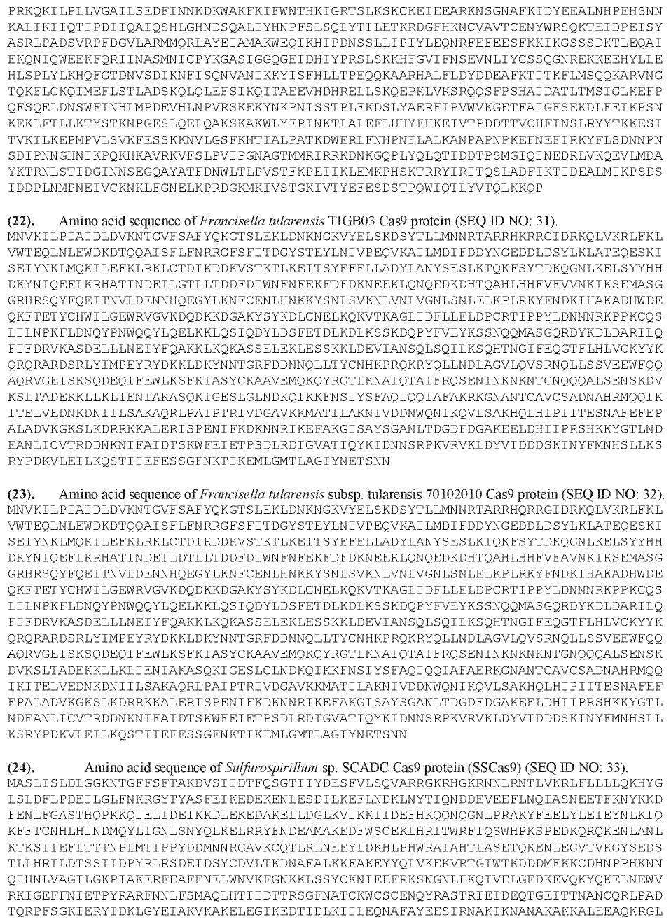

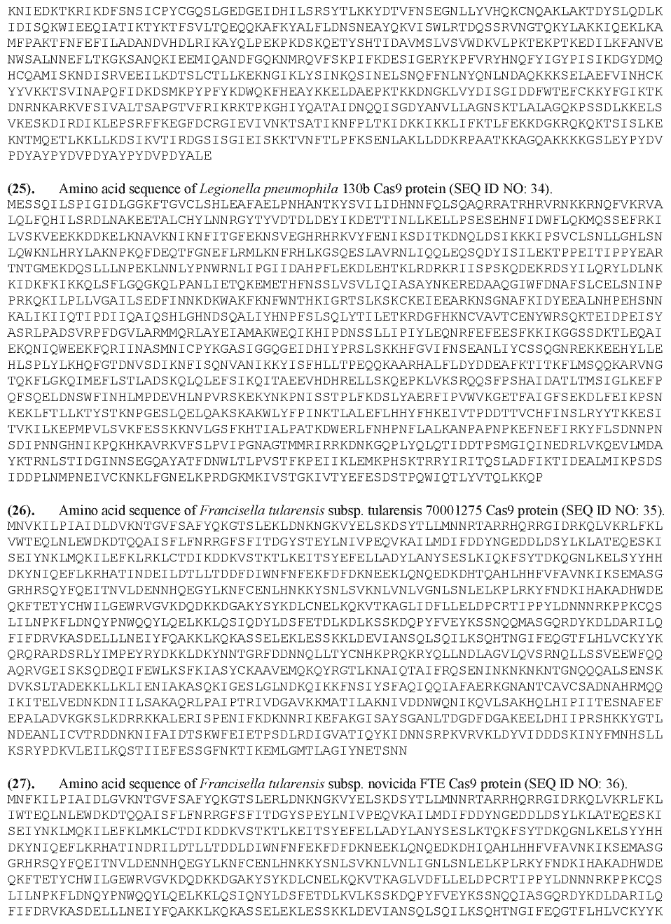

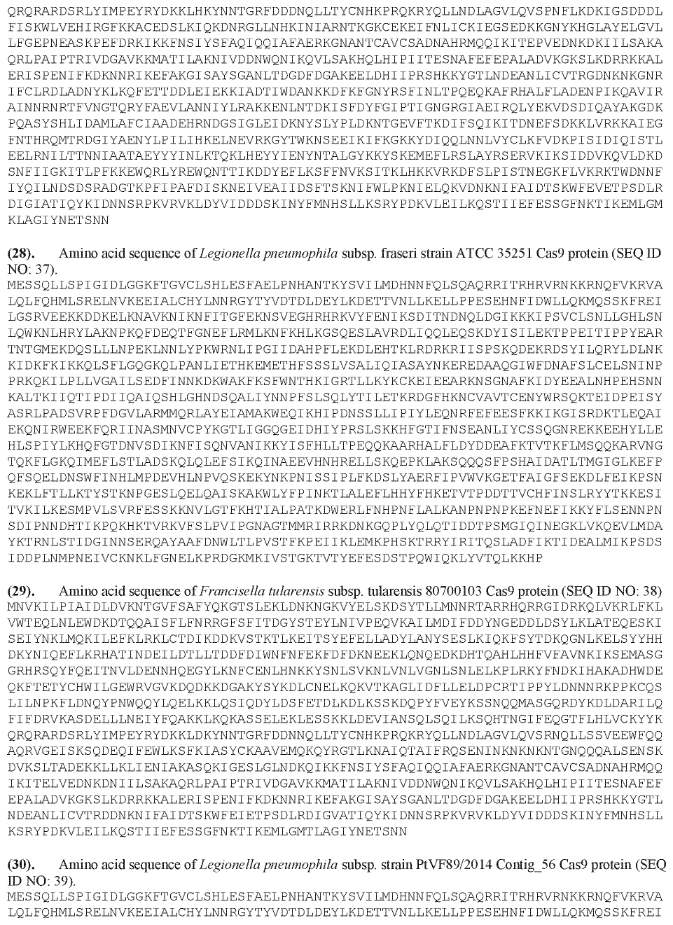

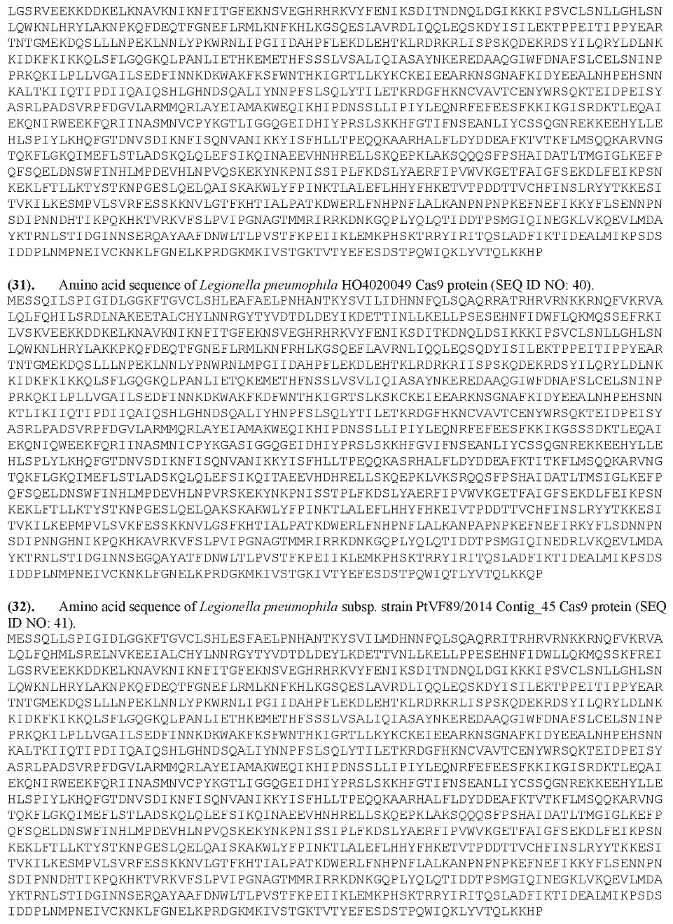

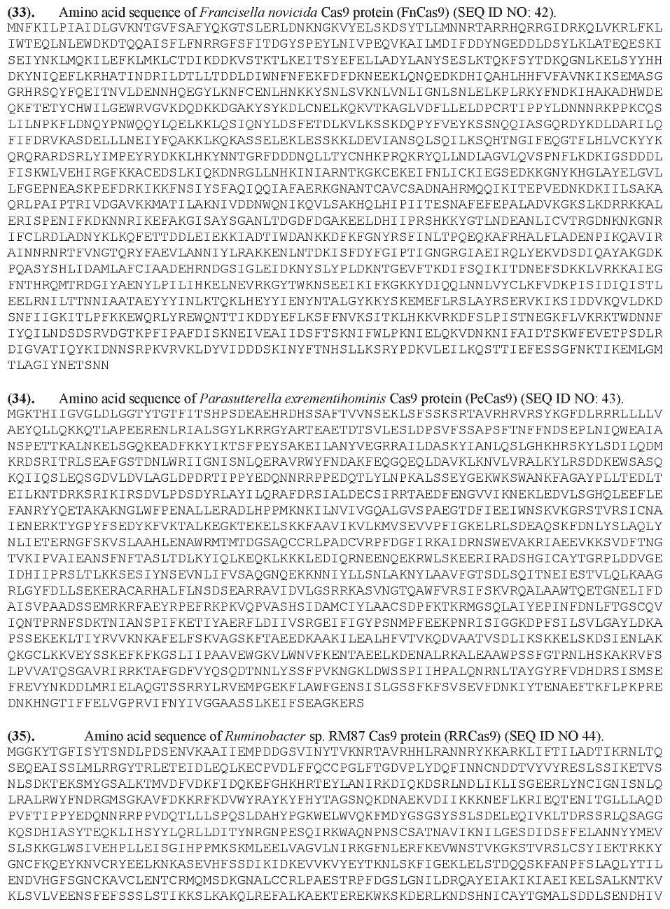

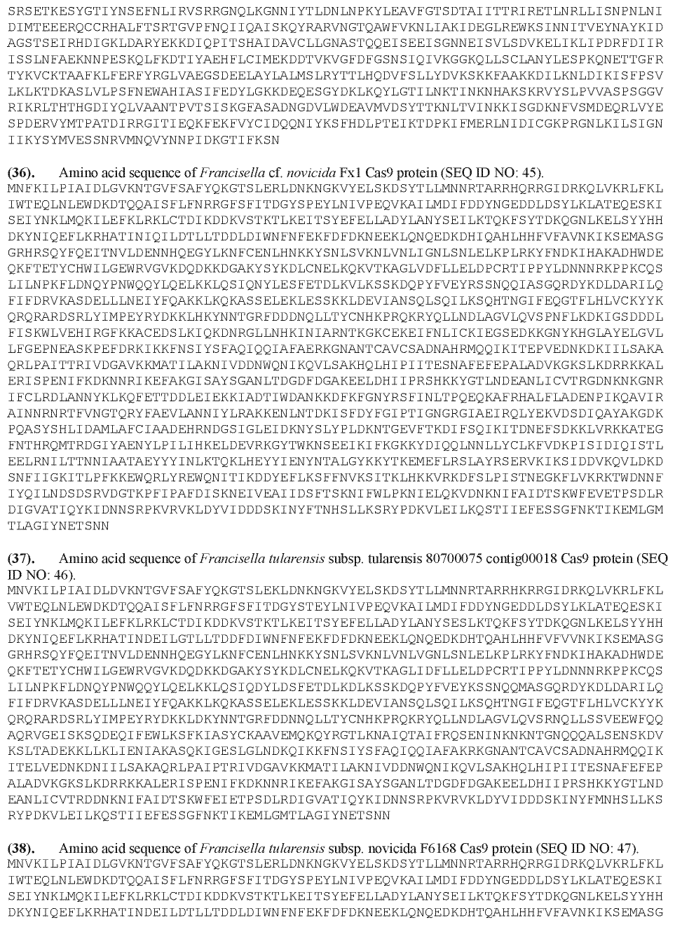

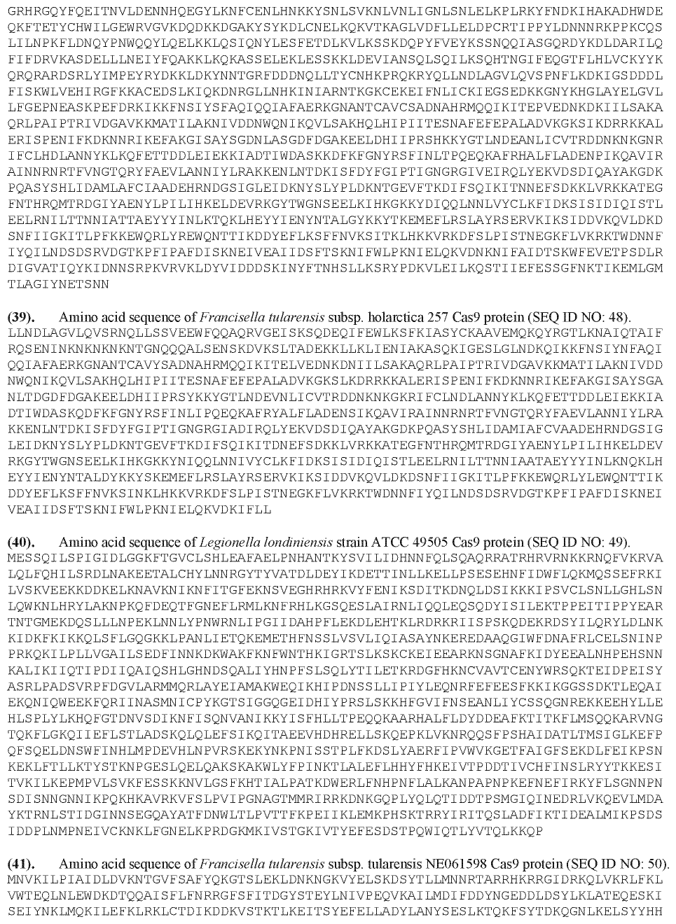

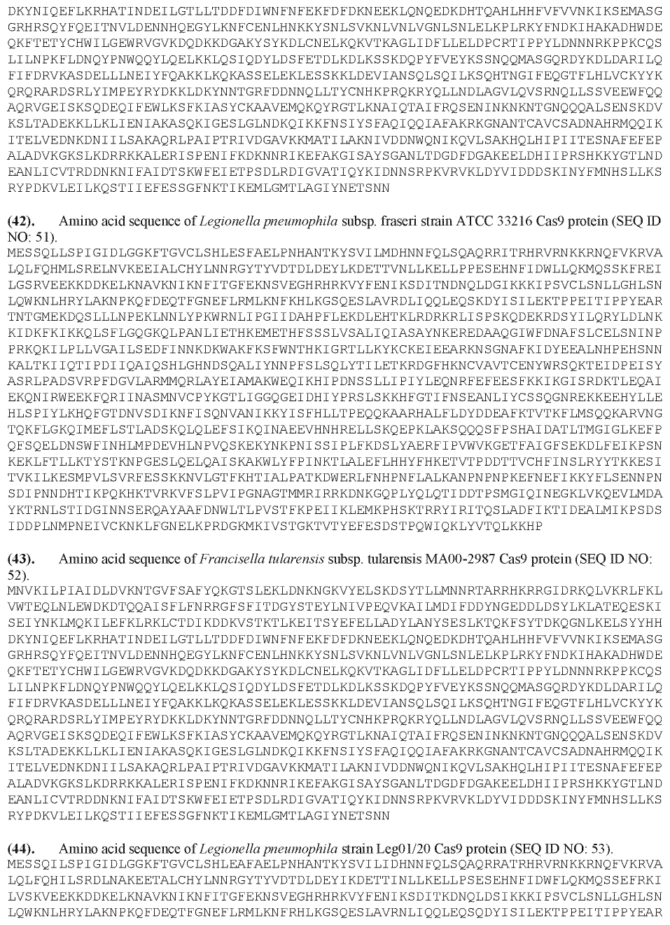

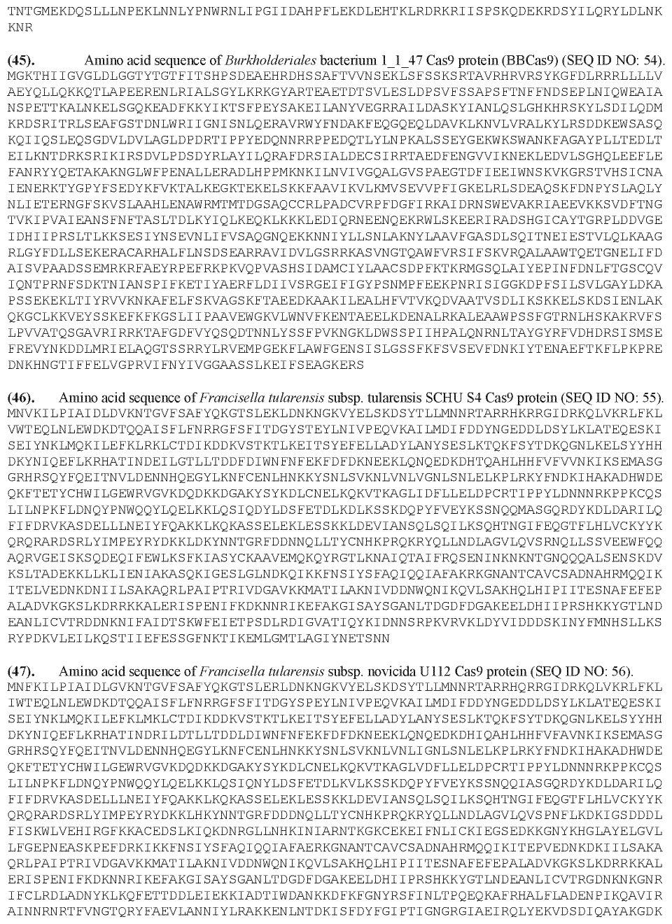

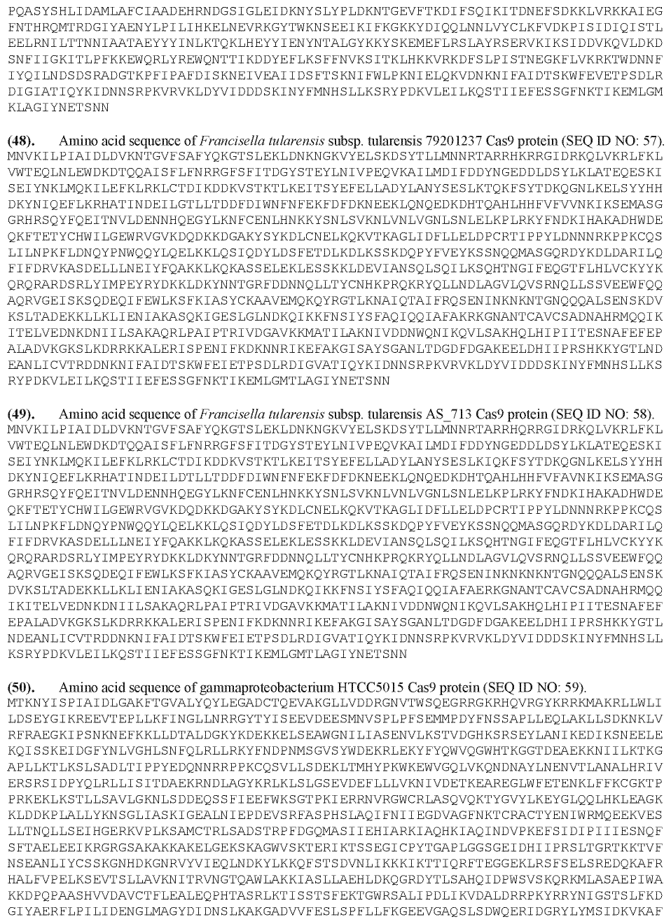

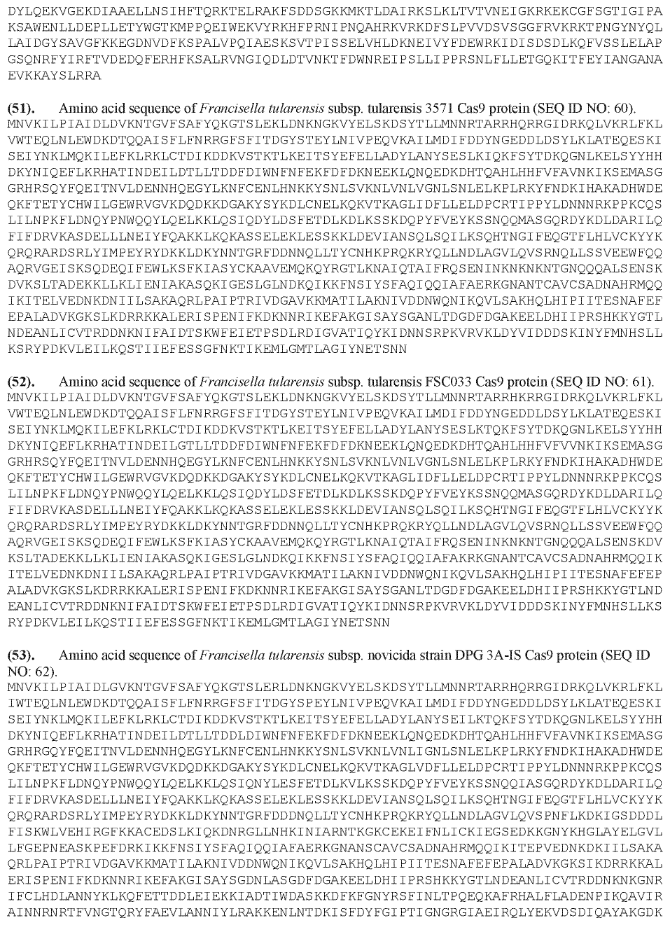

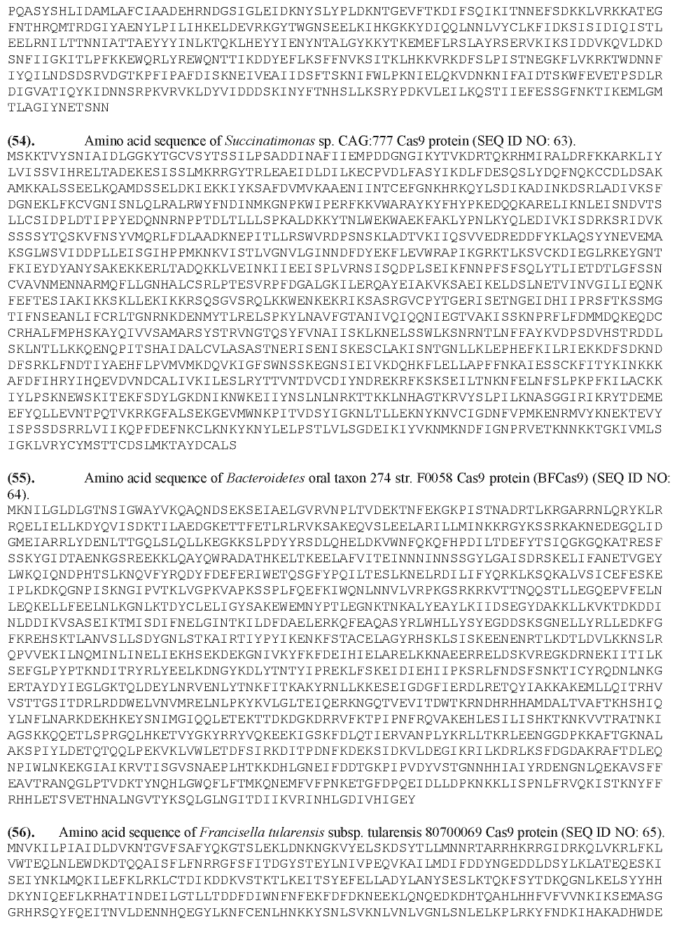

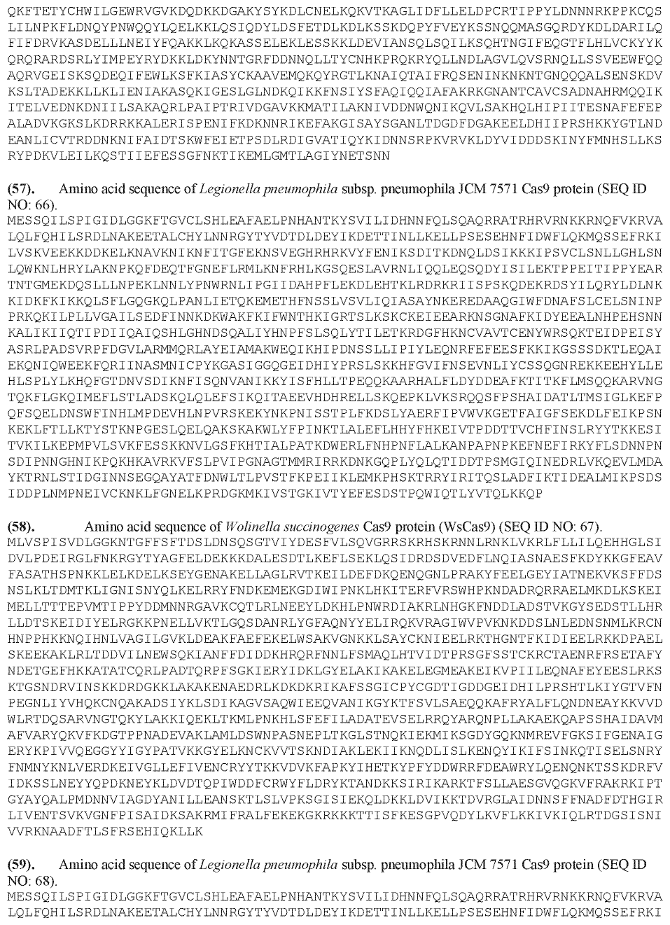

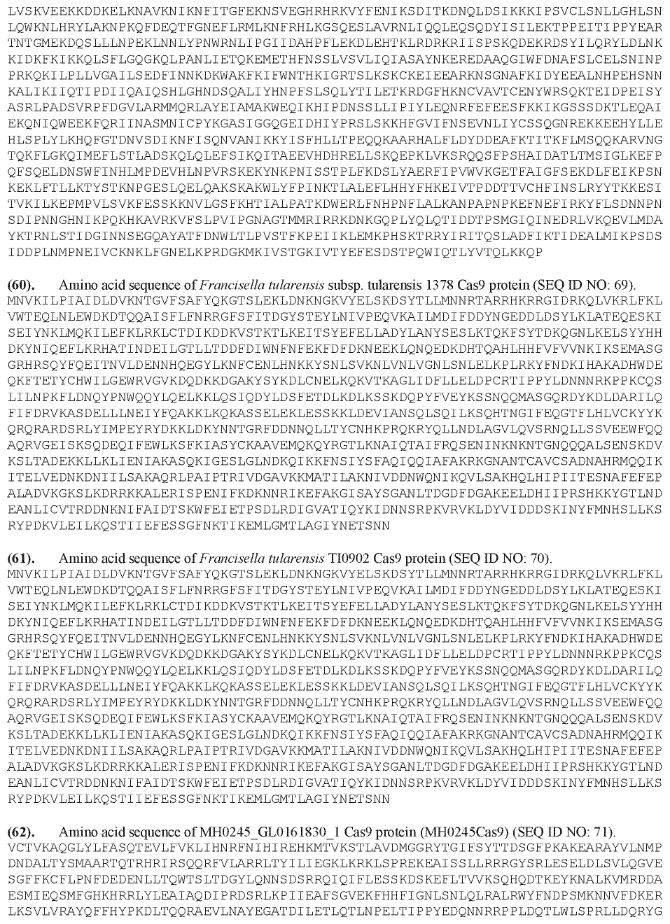

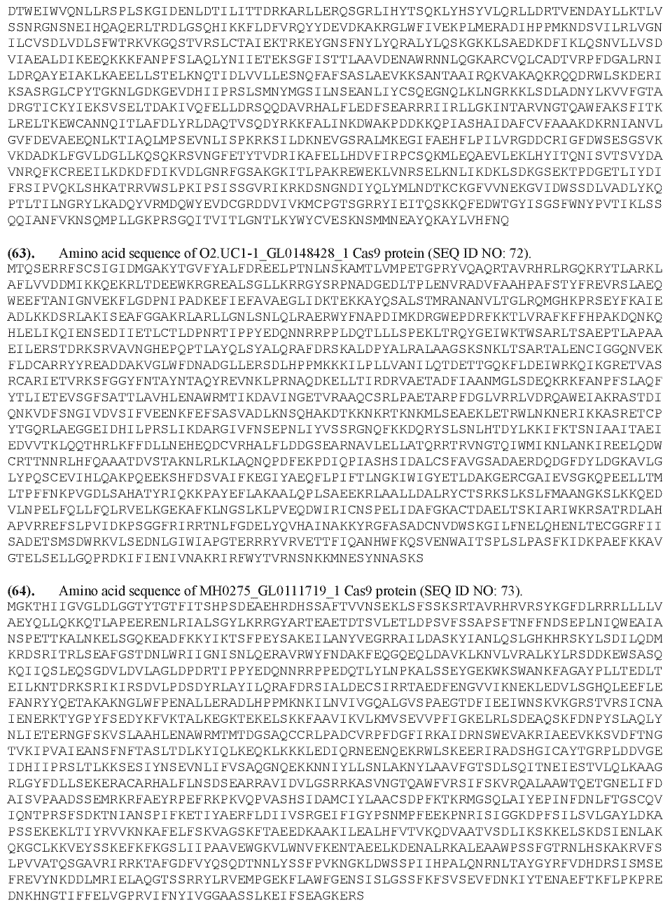

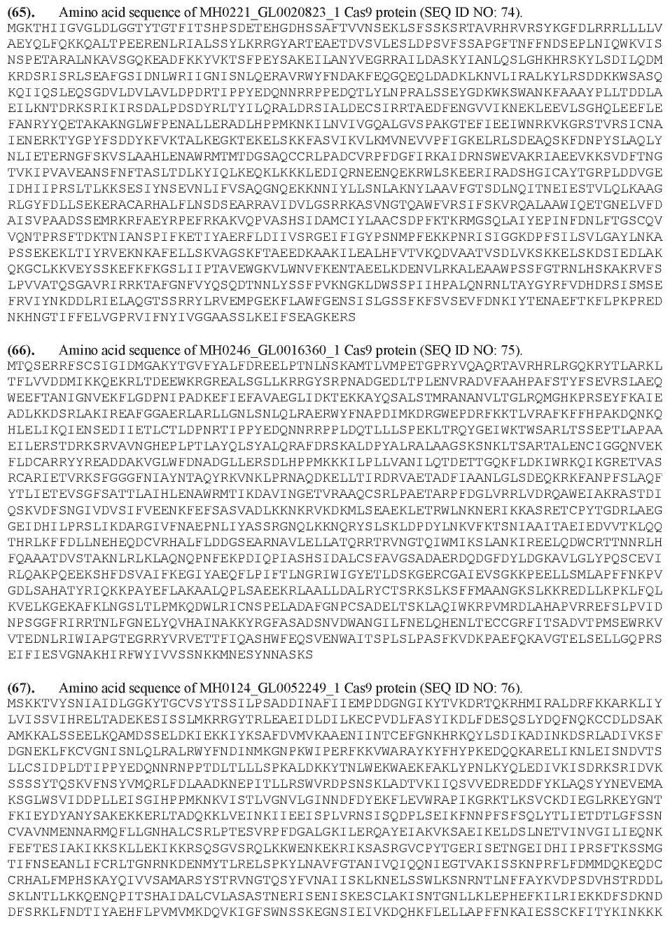

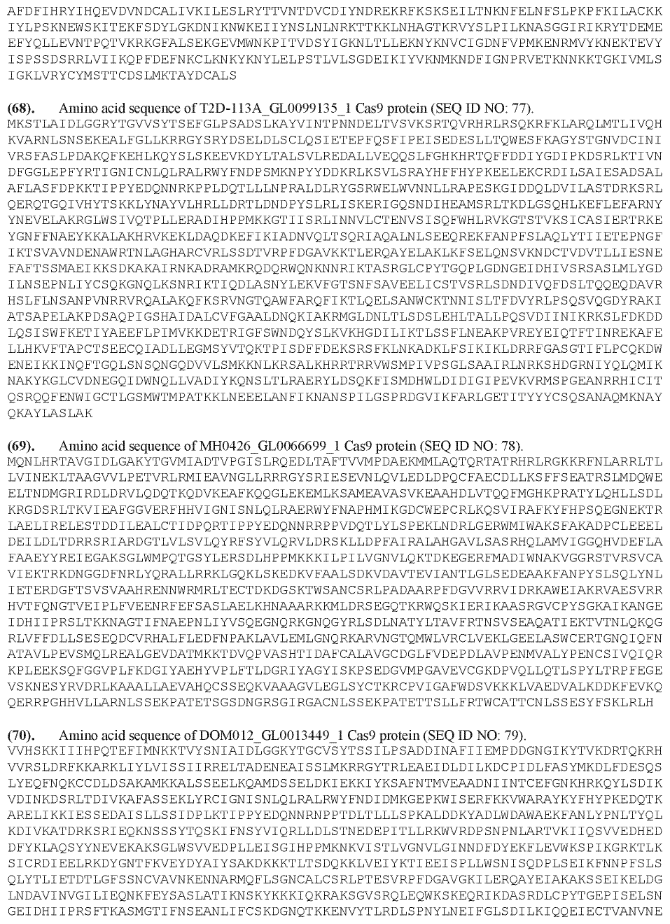

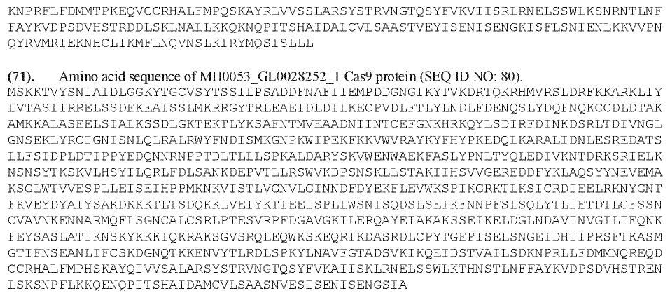

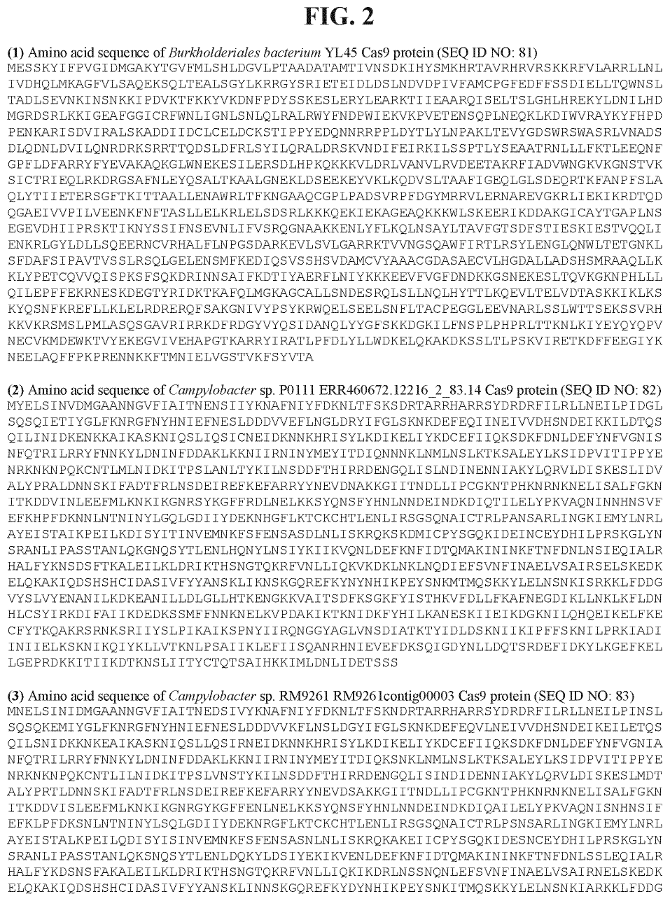

In embodiments, the Cas9 effector protein comprises a polypeptide sequence having at least 95% identity to any one of SEQ ID NOs: 10-97 as shown in FIG. 1 and FIG. 2. In embodiments, the Cas9 effector protein comprises a polypeptide sequence having at least 90% identity to any one of SEQ ID NOs: 10-97 as shown in FIG. 1 and FIG. 2. In embodiments, the Cas9 effector protein comprises a polypeptide sequence having at least 98% identity to any one of SEQ ID NOs: 10-97 as shown in FIG. 1 and FIG. 2. In embodiments, the Cas9 effector protein comprises a polypeptide sequence having at least 99% identity to any one of SEQ ID NOs: 10-97 as shown in FIG. 1 and FIG. 2. In embodiments, the Cas9 effector protein comprises a polypeptide selected from one of SEQ ID NOs: 10-97 as shown in FIG. 1 and FIG. 2.

In embodiments, the Cas9 effector protein comprises a polypeptide sequence having at least 95% identity to SEQ ID NO: 71. In embodiments, the Cas9 effector protein comprises a polypeptide sequence having at least 90% identity to SEQ ID NO: 71. In embodiments, the Cas9 effector protein comprises a polypeptide sequence having at least 98% identity to SEQ ID NO: 71. In embodiments, the Cas9 effector protein comprises a polypeptide sequence having at least 99% identity to SEQ ID NO: 71. In embodiments, the Cas9 effector protein comprises SEQ ID NO: 71.

In embodiments of the protein, the Cas9 effector protein comprises a polypeptide sequence at least 90% identical to SEQ ID NO: 98. In embodiments, the Cas9 effector protein comprises a polypeptide sequence at least 95% identical to SEQ ID NO: 98. In embodiments, the Cas9 effector protein comprises a polypeptide sequence at least 98% identical to SEQ ID NO: 98. In embodiments, the Cas9 effector protein comprises a polypeptide sequence at least 99% identical to SEQ ID NO: 98. In embodiments, the Cas9 effector protein comprises SEQ ID NO: 98.

SEQ ID NO: 98
MKRTADGSEFESPKKKRKVTGTVKSTLAVDMGGRYTGIFSYTTDS
GFPKAKEARAYVLNMPDNDALTYSMAARTQTRHRIRSQQRFVLA
RRLTYILIEGKLKRKLSPREKEAISSLLRRRGYSRLESELDLSVLQGV
ESGFFKCFLPNFDEDENLLTQWTSLTDGYLQNNSDSRRQIQIFLESS
KDSKEFLTVVKSQHQDTKEYKNALKVMRDDAESMIEQSMFGHKH
RRLYLEAIAQDIPRDSRLKPIIEAFSGVEKFHHFIGNLSNLQLRALRW
YFNDPSMKNNVFDKERLKSVLVRAYQFFHYPKDLTQQRAEVLNA
YEGATDILETLQTLNPELTIPPYEDQNNRRPPLDQTLWLSPRLLDQR
YGDTWEIWVQNLLRSPLSKGIDENLDTILITTDRKARLLERQSGRLI
HYTSQKLYHSYVLQRLLDRTVENDAYLLKTLVSSNRGNSNEIHQA
QERLTRDLGSQHIKKFLDFVRQYYDEVDKAKRGLWFIVEKPLMER
ADIHPPMKNDSVILRLVGNILCVSDLVDLSFWTRKVKGQSTVRSLC
TAIEKTRKEYGNSFNYLYQRALYLQSKGKKLSAEDKDFIKLQSNVL
LVSDVIAEALDIKEEQKKKFANPFSLAQLYNIIETEKSGFISTTLAAV
DENAWRNNLQGKARCVQLCADTVRPFDGALRNILDRQAYEIAKL
KAEELLSTELKNQTIDLVVLLESNQFAFSASLAEVKKSANTAAIRQ
KVAKAQKRQQDRWLSKDERIKSASRGLCPYTGKNLGDKGEVDHII
PRSLSMNYMGSILNSEANLIYCSQEGNQLKLNGRKKLSDLADNYL
KVVFGTADRGTICKYIEKSVSELTDAKIVQFELLDRSQQDAVRHAL
FLEDFSEARRRIIRLLGKINTARVNGTQAWFAKSFITKLRELTKEWC
ANNQITLAFDLYRLDAQTVSQDYRKKFALINKDWAKPDDKKQPIA
SHAIDAFCVFAAAKDKRNIANVLGVFDEVAEEQNLKTIAQLMPSE
VNLISPKRKSILDKNEVGSRALMKEGIFAEHFLPILVRGDDCRIGFD
WSESGSVKVKDADKLFGVLDGLLKQSQKRSVNGFETYTVDRIKAF
ELLHDVFIRPCSQKMLEQAEVLEKLHYITQNISVTSVYDAVNRQFK
CREEILKDKDFDIKVDLGNRFGSAKGKITLPAKREWEKLVNRSELK
NLIKDKLSDKGSEKTPDGETLIYDIFRSIPVQKLSHKATRRVWSLPKI
PSISSGVRIKRKDSNGNDIYQLYMLNDTKCKGFVVNEKGVIDWSSD
LVADLYKQPTLTILNGRYLKADQYVRMDQWYEVDCGRDDVIVK
MCPGTSGRRYIEITQSKKQFEDWTGYISGSFWNYPVTIKLSSQQIAN
FVKNSQMPLLGKPRSGQITVITLGNTLKYWYCVESKNSMMNEAYQ
KAYLVHFNQGTPKKKRKV

In some embodiments, the Cas9 effector protein comprises SEQ ID NO: 98 including an amino acid modification at one or more position of R1336, R1389, R668, N1164, N1265, N1300, N1412, N347, N348, N562, N565, N618, N651, D1266, D309, D345, D487, D607, D30, Q1129, Q1381, Q624, Q661, Q713, Q734, E1032, E1409, E436, E610, E611, E691, E697, G1245, G1335, H777, 11242, L125, L1162, L1264, L1299, K1031, K443, K490, K615, K656, F1035, F620, F636, F670, S1243, S1334, S1380, S1410, S1413, S634, S638, S711, S1006, S1017, T1267, T1333, T551, T639, T639, T640, T666, T897, Y1338, Y343, Y566, V1273, V1274, V486, V644, V660, V667, V736 of SEQ ID NO:98, or combinations thereof.

In some embodiments, the amino acid modification includes one or more of the following mutations R668A, N1164R, N1265R, N1300R, N1412R, N347R, N348R, N562R, N565R, N618R, N651A, N651R, D1266R, D309R, D345R, D487R, D607R, Q1129R, Q1381A, Q1381A, Q1381R, Q624R, Q661A, Q661R, Q713R, Q734R, E1032G, E1032R, E1409A, E1409R, E436R, E610R, E611R, E691R, E697R, G1245R, G1335R, H777A, 11242S, L125R, L125Y, L1162S, L1264S, L1299S, K1031R, K443R, K490R, K615R, K656R, F1035R, F620R, F636R, F670Y, S1243R, S1334A, S1334A, S1334R, S1380R, S1410R, S1413R, S634R, S638A, S638R, S711R, S1006R, S1017R, T1267A, T1267R, T1333A, T1333R, T551R, T639A, T639R, T640R, T666R, T897R, Y1338A, Y1338R, Y343R, Y566R, V1273S, V1274S, V486R, V644R, V660R, V660Y, V667R, V667S, V736R or V736Y. In some embodiments, the amino acid modification includes one or more of the following mutations N1164R, N1265R, N1300R, N1412R, N347R, N651A, D1266R, D309R, D345R, D487R, D607R, Q1129R, Q1381A, Q1381A, Q1381R, Q661A, Q713R, Q734R, E1032G, E1032R, E1409A, E436R, E611R, E691R, E697R, G1335R, L125R, L1264S, L1299S, K1031R, K490R, K615R, K656R, F636R, S1334A, S1334A, S1334R, S1380R, S1410R, S1413R, S634R, S638R, S711R, S1006R, S1017R, T1267A, T1267R, T551R, Y1338A, Y1338R, V1273S, V1274S, V486R, V644R, V736R or V736Y. In some embodiments, the amino acid modification includes one or more of the following mutations N1265R, N1300R, N1412R, D1266R, E436R, G1335R, S1334R, S1380R, S1017R, T1267R, V736R or V736Y.

In some embodiments, the amino acid modification results in an increased binding affinity between Cas9 effector protein and DNA.

### CRISPR-Cas Systems

In embodiments, the present disclosure provides a CRISPR-Cas system comprising a Cas9 effector protein having enhanced stability.

In general, a CRISPR or CRISPR-Cas or CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide polynucleotide is designed to target, e.g. have complementarity, where hybridization between a target sequence and a guide polynucleotide promotes the formation of a CRISPR complex. The section of the guide polynucleotide through which complementarity to the target sequence can be important for cleavage activity is referred to herein as the guide sequence. A target sequence may comprise any polynucleotide, such as DNA or RNA polynucleotides and can be located within a target locus of interest. In embodiments, a target sequence is located in the nucleus or cytoplasm of a cell. In embodiments, the target sequence is located on the chromosome (TSC). In embodiments, the target sequence is located on a vector (TSV).

In embodiments, the present disclosure provides a CRISPR-Cas system comprising:
a) a Cas9 effector protein comprising: i) a first nuclear localization signal attached to the N-terminus of the Cas9 effector protein; and ii) a second nuclear localization signal attached to the C-terminus of the Cas9 effector protein; and
b) a guide polynucleotide comprising a guide sequence and forms a complex with the Cas9 effector protein, wherein the guide sequence is capable of hybridizing with a target sequence in a eukaryotic cell.

In embodiments, the present disclosure provides a CRISPR-Cas system comprising:
a) a nucleic acid sequence encoding a Cas9 effector protein comprising: i) a first nuclear localization signal attached to the N-terminus of the Cas9 effector protein; and ii) a second nuclear localization signal attached to the C-terminus of the Cas9 effector protein; and
b) a nucleic acid sequence encoding a guide polynucleotide that comprises a guide sequence and forms a complex with the Cas9 effector protein, wherein the guide sequence is capable of hybridizing with a target sequence in a eukaryotic cell.

In embodiments of this system, the nucleotide sequences of (a) and (b) are under control of the same promoter. In embodiments, the nucleotide sequences of (a) and (b) are under control of different promoters.

As used herein, “promoter,” “promoter sequence,” or “promoter region” refers to a DNA regulatory region/sequence capable of binding RNA polymerase and involved in initiating transcription of a downstream coding or non-coding sequence. In some examples of the present disclosure, the promoter sequence includes the transcription initiation site and extends upstream to include the minimum number of bases or elements used to initiate transcription at levels detectable above background. In embodiments, the promoter sequence includes a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present disclosure.

In embodiments, the nucleotide sequences of (a) and (b) are under control of a eukaryotic promoter. In embodiments, the nucleotide sequences of (a) and (b) are under control of two different eukaryotic promoters. In embodiments, at least one of the eukaryotic promoters is a promoter that is active in human induced pluripotent stem cells.

In embodiments at least one of the eukaryotic promoters is EF1alpha (EF1a). In embodiments at least one of the eukaryotic promoters is human cytomegalovirus (CMV) promoter. In embodiments at least one of the eukaryotic promoters is doxycycline regulatable promoter TRE3G. In embodiments, the nucleotide sequences of (a) and (b) are under control of a bacterial promoter. In embodiments, the nucleotide sequences of (a) and (b) are under control of two different bacterial promoters. In embodiments, the nucleotide sequences of (a) and (b) are under control of a viral promoter. In embodiments, the nucleotide sequences of (a) and (b) are under control of two different viral promoters.

In embodiments, the nucleic acid sequences of (a) and (b) are in a single vector. In embodiments, the nucleic acid sequences of (a) and (b) are in separate vectors.

In embodiments, the present disclosure provides a CRISPR-Cas system comprising one or more vectors comprising:
a) a regulatory element operably linked to one or more nucleotide sequences encoding a Cas9 effector protein comprising: i) a first nuclear localization signal attached to the N-terminus of the Cas9 effector protein; and ii) a second nuclear localization signal attached to the C-terminus of the Cas9 effector protein; and
b) a guide polynucleotide that comprises a guide sequence and forms a complex with the Cas9 effector protein, wherein the guide sequence is capable of hybridizing with a target sequence in a eukaryotic cell.

In embodiments of this system, the regulatory element is a eukaryotic regulatory element. In embodiments of this system, the regulatory element is a prokaryotic regulatory element.

In embodiments, the nucleotide encoding a Cas9 effector protein and a guide polynucleotide is on a single vector. In embodiments, a nucleotide encoding a Cas9 effector protein, a guide polynucleotide (or nucleotide that can be transcribed into a guide polynucleotide), and a tracrRNA are on a single vector. In embodiments, the nucleotide encoding a Cas9 effector protein, a guide polynucleotide (or nucleotide that can be transcribed into a guide polynucleotide), a tracrRNA, and a direct repeat sequence are on a single vector. In embodiments, the vector is an expression vector. In embodiments, the vector is a mammalian expression vector. In embodiments, the vector is a human expression vector. In embodiments, the vector is a plant expression vector.

In embodiments, the nucleotide encoding a Cas9 effector protein and a guide polynucleotide is a single nucleic acid molecule. In embodiments, the nucleotide encoding a Cas9 effector protein, a guide polynucleotide, and a tracrRNA is a single nucleic acid molecule. In embodiments, the nucleotide encoding a Cas9 effector protein, a guide polynucleotide, a tracrRNA, and a direct repeat sequence is a single nucleic acid molecule. In embodiments, the single nucleic acid molecule is an expression vector. In embodiments, the single nucleic acid molecule is a mammalian expression vector. In embodiments, the single nucleic acid molecule is a human expression vector. In embodiments, the single nucleic acid molecule is a plant expression vector.

“Operably linked” means that the nucleotide of interest, i.e., the nucleotide encoding a Cas9 effector protein, is linked to the regulatory element in a manner that allows for expression of the nucleotide sequence. Thus, In embodiments, the vector is an expression vector.

In embodiments, the regulatory element is a promoter. In embodiments, the regulatory element is a bacterial promoter. In embodiments, the regulatory element is a viral promoter.

In embodiments, the regulatory element is a eukaryotic regulatory element, i.e., a eukaryotic promoter. In embodiments, the eukaryotic regulatory element is a mammalian promoter.

In embodiments of any of the above systems, the first nuclear localization signal is a monopartite nuclear localization signal. In embodiments, the first nuclear localization signal is a bipartite nuclear localization signal. In embodiments, the second nuclear localization signal is a monopartite nuclear localization signal. In embodiments, the second nuclear localization signal is a bipartite nuclear localization signal.

In embodiments, the first and second nuclear localization signals can both be monopartite, both be bipartite or can be a mixture of monopartite and bipartite. In embodiments, the first nuclear localization signal is a bipartite nuclear localization signal and the second nuclear localization signal is a monopartite nuclear localization signal. In embodiments, the first nuclear localization signal is a monopartite nuclear localization signal and the second nuclear localization signal is a bipartite nuclear localization signal.

In embodiments, the first nuclear localization signal is a monopartite nuclear localization signal and the second nuclear localization signal is a monopartite nuclear localization signal. In embodiments, the first nuclear localization signal is a bipartite nuclear localization signal and the second nuclear localization signal is a bipartite nuclear localization signal.

In embodiments of any of the above systems, the monopartite nuclear localization signal is a monopartite nuclear localization signal known in the art. In embodiments, the monopartite nuclear localization signal is one of the monopartite nuclear localization signals listed in Table 1 above (SEQ ID NOs: 1-6), or combinations thereof.

In embodiments of any of the above systems, the bipartite nuclear localization signal is a bipartite nuclear localization signal known in the art. In embodiments, the bipartite nuclear localization signal is a classical bipartite nuclear localization signal. In embodiments, the bipartite nuclear localization signal is one of the bipartite nuclear localization signals listed in Table 2 above (SEQ ID NOs: 7-9), or combinations thereof.

In embodiments of any of the above systems, the first nuclear localization signal is classic bipartite nuclear localization signal (SEQ ID NO: 7) and the second nuclear localization signal is SV40 Large T-Antigen nuclear localization signal (SEQ ID NO: 1).

In embodiments of any of the above systems, the first nuclear localization signal is directly attached to the Cas9 effector protein. In embodiments, the first nuclear localization signal is attached to the Cas9 effector protein via a linker. In embodiments, the second nuclear localization signal is directly attached to the Cas9 effector protein. In embodiments, the second nuclear localization signal is attached to the Cas9 effector protein via a linker.

In embodiments where a linker is used, the linker is a peptide linker having from 2 to 30 residues. In embodiments, linker is a peptide linker having from 2 to 20 residues. In embodiments, linker is a peptide linker having from 2 to 15 residues. In embodiments, linker is a peptide linker having from 2 to 10 residues. In embodiments, linker is a peptide linker having from 2 to 5 residues. In embodiments, the linker is a substituted or unsubstituted C₂-C₂₀ alkyl, alkene or alkynyl chain.

In embodiments of any of the above systems, the Cas9 effector protein comprises more than one copy of a nuclear localization signal on its N-terminus. In embodiments, the Cas9 effector protein comprises more than one type of nuclear localization signal on its N-terminus. In embodiments, the Cas9 effector protein comprises more than one copy of a nuclear localization signal on its C-terminus. In embodiments, the Cas9 effector protein comprises more than one type of nuclear localization signal on its C-terminus.

In embodiments of any of the above systems, the protein comprises two copies of the first nuclear localization signal. In embodiments, the protein comprises three copies of the first nuclear localization signal. In embodiments, the protein comprises two copies of the second nuclear localization signal. In embodiments, the protein comprises three copies of the second nuclear localization signal.

In embodiments of any of the above systems, the Cas9 portion of the Cas9 protein comprising a first and a second nuclear localization signal can be derived from any Cas9 effector domain known in the art. In embodiments, the Cas9 effector protein is derived from a bacterial species having a Type II-B CRISPR system. Examples of suitable Type II-B Cas9 proteins are described above. In embodiments, the Cas9 portion comprises a domain that matches the TIGR03031 protein family with an E-value cut-off of 1E-5. In embodiments, the site-specific nuclease comprises a domain that matches the TIGR03031 protein family with an E-value cut-off of 1E-10.

In embodiments of any of the above systems, the Cas9 effector protein comprises a polypeptide sequence having at least 95% identity to any one of SEQ ID NOs: 10-97 as shown in FIG. 1 and FIG. 2. In embodiments, the Cas9 effector protein comprises a polypeptide sequence having at least 90% identity to any one of SEQ ID NOs: 10-97 as shown in FIG. 1 and FIG. 2. In embodiments, the Cas9 effector protein comprises a polypeptide sequence having at least 98% identity to any one of SEQ ID NOs: 10-97 as shown in FIG. 1 and FIG. 2. In embodiments, the Cas9 effector protein comprises a polypeptide sequence having at least 99% identity to any one of SEQ ID NOs: 10-97 as shown in FIG. 1 and FIG. 2. In embodiments, the Cas9 effector protein comprises a polypeptide selected from one of SEQ ID NOs: 10-97 as shown in FIG. 1 and FIG. 2.

In embodiments of any of the above systems, the Cas9 effector protein comprises a polypeptide sequence having at least 95% identity to SEQ ID NO: 71. In embodiments, the Cas9 effector protein comprises a polypeptide sequence having at least 90% identity to SEQ ID NO: 71. In embodiments, the Cas9 effector protein comprises a polypeptide sequence having at least 98% identity to SEQ ID NO: 71. In embodiments, the Cas9 effector protein comprises a polypeptide sequence having at least 99% identity to SEQ ID NO: 71. In embodiments, the Cas9 effector protein comprises SEQ ID NO: 71.

In embodiments of any of the above systems, the Cas9 effector protein comprises a polypeptide sequence at least 90% identical to SEQ ID NO: 98. In embodiments, the Cas9 effector protein comprises a polypeptide sequence at least 95% identical to SEQ ID NO: 98. In embodiments, the Cas9 effector protein comprises a polypeptide sequence at least 98% identical to SEQ ID NO: 98. In embodiments, the Cas9 effector protein comprises a polypeptide sequence at least 99% identical to SEQ ID NO: 98. In embodiments, the Cas9 effector protein comprises SEQ ID NO: 98.

In other embodiments of any of the above systems, the Cas9 portion of the Cas9 effector protein comprises a dCas9, i.e., a deactivated or “dead” Cas9 lacking DNA double strand break activity. In embodiments, the dCas9 can be fused with other active domains, such as transcriptional regulators, epigenetic regulator proteins or fluorescent proteins as described elsewhere herein. In embodiments where a dCas9 is fused to another active domain, the nuclear localization signals described herein are present at the N-terminus and C-terminus of the overall Cas9 effector protein construct.

In other embodiments of any of the above systems, the Cas9 portion of the Cas9 effector protein comprises a Cas9 nickase, i.e., a Cas9 protein that only cleaves one strand of the DNA double strand. In embodiments, the Cas9 nickase can be fused with other active domains, such as transcriptional regulators, epigenetic regulator proteins or fluorescent proteins as described elsewhere herein. In embodiments where a Cas9 nickase is fused to another active domain, the nuclear localization signals described herein are present at the N-terminus and C-terminus of the overall Cas9 effector protein construct.

The systems and methods described herein can comprise a guide polynucleotide. In embodiments, the guide polynucleotide is an RNA. The RNA that binds to CRISPR-Cas9 components and targets them to a specific location within the target DNA is referred to herein as “guide RNA,” “gRNA,” or “small guide RNA” and may also be referred to herein as a “DNA-targeting RNA.” A guide polynucleotide, e.g., guide RNA, comprises at least two nucleotide segments: at least one “DNA-binding segment” and at least one “polypeptide-binding segment.” By “segment” is meant a part, section, or region of a molecule, e.g., a contiguous stretch of nucleotides of guide polynucleotide molecule. The definition of “segment,” unless otherwise specifically defined, is not limited to a specific number of total base pairs.

In embodiments, the DNA-binding segment of the guide polynucleotide hybridizes with a target sequence in a eukaryotic cell, but not a sequence in a bacterial cell. A sequence in a bacterial cell, as used herein, refers to a polynucleotide sequence that is native to a bacterial organism, i.e., a naturally-occurring bacterial polynucleotide sequence, or a sequence of bacterial origin. For example, the sequence can be a bacterial chromosome or bacterial plasmid, or any other polynucleotide sequence that is found naturally in bacterial cells.

In embodiments, the polypeptide-binding segment of the guide polynucleotide binds to a Cas9 effector protein having enhanced stability as described herein.

In embodiments, the guide polynucleotide is 10 to 150 nucleotides. In embodiments, the guide polynucleotide is 20 to 120 nucleotides. In embodiments, the guide polynucleotide is 30 to 100 nucleotides. In embodiments, the guide polynucleotide is 40 to 80 nucleotides. In embodiments, the guide polynucleotide is 50 to 60 nucleotides. In embodiments, the guide polynucleotide is 10 to 35 nucleotides. In embodiments, the guide polynucleotide is 15 to 30 nucleotides. In embodiments, the guide polynucleotide is 20 to 25 nucleotides.

The guide polynucleotide, e.g., guide RNA, can be introduced into the target cell as an isolated molecule, e.g., RNA molecule, or is introduced into the cell using an expression vector containing DNA encoding the guide polynucleotide, e.g., guide RNA.

The “DNA-binding segment” (or “DNA-targeting sequence”) of the guide polynucleotide, e.g., guide RNA, comprises a nucleotide sequence that is complementary to a specific sequence within a target DNA.

The guide polynucleotide, e.g., guide RNA, of the present disclosure can include a polypeptide-binding sequence/segment. The polypeptide-binding segment (or “protein-binding sequence”) of the guide polynucleotide, e.g., guide RNA, interacts with the polynucleotide-binding domain of a Cas protein of the present disclosure. Such polypeptide-binding segments or sequences are known to those of skill in the art, e.g., those disclosed in U.S. patent application publications 2014/0068797, 2014/0273037, 2014/0273226, 2014/0295556, 2014/0295557, 2014/0349405, 2015/0045546, 2015/0071898, 2015/0071899, and 2015/0071906, the disclosures of which are incorporated herein in their entireties.

In some embodiments, the polypeptide-binding segment has been modified to improve binding to a polypeptide of the invention. Methods modify polypeptide-binding segments to improve binding are described in Riesenberg et al. (Nature Communications, 2021) and references therein. Optimized polypeptide-binding segments of guide RNAs suitable for SEQ ID NO. 98 are shown in Table 3 as SEQ ID NO: 100-107. SEQ ID NO:99 is a polypeptide-binding segment sequence suitable for SEQ ID NO:98 before optimization. In some embodiments, the guide RNA comprises a sequence selected from SEQ ID NO. 99, SEQ ID NO. 100, SEQ ID NO. 101, SEQ ID NO. 102, SEQ ID NO. 103, SEQ ID NO. 104, SEQ ID NO. 105, SEQ ID NO. 106, or SEQ ID NO. 107.

TABLE 3

| | |
|---|---|
| SEQ ID | GUUUCAGUUAUUCGUGAAAACGAAUGAAGUCACUCUUAAAGU |
| NO: 99 | GAGCUGAAAUCACUAAAAAUUAAGAUUGAACCCGGCUACUGA |
| | CUCUGUCAUCCGGGUUUACUUAUUU |
| SEQ ID | AUGUCACCUCCAAUGACUAGGGGUUUCAGUUAUUCGUGAAAA |
| NO: 100 | CGAAUGAAGUCACUCUUAAAGUGAGCUGAAAUCACUAAAAAU |
| | UAAGAUUGAACCCGGCUACUGACUCUGUCAUCCGGGUUU |
| SEQ ID | AUGUCACCUCCAAUGACUAGGGGUUUCAGUUAUUCGUGAAAA |
| NO: 101 | CGAAUGAAGUCACUCUUAAAGUGAGCUGAAAUCACUAAAAAU |
| | UAAGAUUGAACCCGGCUACUGACUCUGUCAUCCGGGUU |
| SEQ ID | AUGUCACCUCCAAUGACUAGGGGUUUCAGUUAUUCGUGAAAA |
| NO: 102 | CGAAUGAAGUCACUCUUAAAGUGAGCUGAAAUCACUAAAAAU |
| | UAAGAUUGAACCCGGCUACUGACUCUGUCAUCCGGGU |
| SEQ ID | AUGUCACCUCCAAUGACUAGGGGUUUCAGUUAUUCGUGAAAA |
| NO: 103 | CGAAUGAAGUCACUCUUAAAGUGAGCUGAAAUCACUAAAAAU |
| | UAAGAUUGAACCCGGCUACUGACUCUGUCAUCCGGG |
| SEQ ID | AUGUCACCUCCAAUGACUAGGGGUUUCAGUUAGAAAUGAAGU |
| NO: 104 | CACUCUUAAAGUGAGCUGAAAUCACUAAAAAUUAAGAUUGAA |
| | CCCGGCUACUGACUCUGUCAUCCGGGUUU |
| SEQ ID | AUGUCACCUCCAAUGACUAGGGGUUUCAGUUAUGAAGUCACU |
| NO: 105 | CUUAAAGUGAGCUGAAAUCACUAAAAAUUAAGAUUGAACCCG |
| | GCUACUGACUCUGUCAUCCGGGUUU |
| SEQ ID | AUGUCACCUCCAAUGACUAGGGGUUUCAGUUAUGAAGUCACU |
| NO: 106 | GAAAAGUGAGCUGAAAUCACUAAAAAUUAAGAUUGAACCCGG |
| | CUACUGACUCUGUCAUCCGGGUUU |
| SEQ ID | AUGUCACCUCCAAUGACUAGGGGUUUCAGUUGAAAGAAGUCA |
| NO: 107 | CUCUUAAAGUGAGCUGAAAUCACUAAAAAUUAAGAUUGAACC |
| | CGGCUACUGACUCUGUCAUCCGGGUUU |

In embodiments of the present disclosure, the Cas9 effector protein and the guide polynucleotide can form a complex. A “complex” is a group of two or more associated nucleic acids and/or polypeptides. In embodiments, a complex is formed when all the components of the complex are present together, i.e., a self-assembling complex. In embodiments, a complex is formed through chemical interactions between different components of the complex such as, for example, hydrogen-bonding. In embodiments, a guide polynucleotide forms a complex with a Cas9 effector protein through secondary structure recognition of the guide polynucleotide by the Cas9 effector protein. In embodiments, a Cas9 effector protein is inactive, i.e., does not exhibit nuclease activity, until it forms a complex with a guide polynucleotide. Binding of guide RNA induces a conformational change in Cas9 effector protein to convert the Cas9 effector protein from the inactive form to an active, i.e., catalytically active, form.

In embodiments of any of the above systems, the guide sequence is from 19 to 30 bases in length. In embodiments, the guide sequence is from 19 to 25 bases in length. In embodiments, the guide sequence is from 21 to 26 bases in length.

In embodiments of any of the above systems, the guide polynucleotide further comprises a tracrRNA sequence. A “tracrRNA,” or trans-activating CRISPR-RNA, forms an RNA duplex with a pre-crRNA, or pre-CRISPR-RNA, and is then cleaved by the RNA-specific ribonuclease RNase III to form a crRNA/tracrRNA hybrid. In embodiments, the guide RNA comprises the crRNA/tracrRNA hybrid. In embodiments, the tracrRNA component of the guide RNA activates the Cas9 effector protein.

In embodiments of the systems disclosed herein, the Cas9 effector protein, guide polynucleotide, and tracrRNA are capable of forming a complex.

In embodiments of any of the above systems, the Cas9 effector protein generates cohesive ends. In embodiments, the cohesive ends generated by the Cas9 effector protein comprise a 5′ overhang. In embodiments, the cohesive ends generated by the Cas9 effector protein comprise a 3′ overhang. In embodiments, the cohesive ends comprise a single-stranded polynucleotide overhang of 1 to 10 nucleotides. In embodiments, the cohesive ends comprise a single-stranded polynucleotide overhang of 2 to 6 nucleotides. In embodiments, the cohesive ends comprise a single-stranded polynucleotide overhang of 3 to 5 nucleotides.

In embodiments, the Cas9 effector protein prefers cohesive ends with multiple nucleotides on the 5′ end. In embodiments, the Cas9 effector protein prefers cohesive ends with 3 nucleotides on the 5′ end. In embodiments, the Cas9 effector protein prefers cohesive ends with 2, 3, 4, 5 or 6 nucleotides on the 5′ end. In embodiments, this preference is in contrast to traditionally used S. pyogenes Cas9 (SpCas9), which prefers a single nucleotide 5′ cohesive end.

In embodiments, the presence of a single nucleotide 5′ cohesive end can be used to direct insertion of a nucleic acid of interest in a specific orientation. In embodiments, the presence of three nucleotides on the 5′ cohesive end can be used to direct insertion of a nucleic acid of interest in a specific orientation. In embodiments, the presence of two, three, four, five or six nucleotides on the 5′ cohesive end can be used to direct insertion of a nucleic acid of interest in a specific orientation.

### Cells

In embodiments, the present disclosure provides a eukaryotic cell comprising a Cas9 effector protein as described herein. In embodiments, the present disclosure also provides a eukaryotic cell comprising a system comprising a Cas9 effector protein as described herein.

In embodiments, the eukaryotic cell is an animal or human cell. In embodiments, the eukaryotic cell is a human or rodent or bovine cell line or cell strain. Examples of such cells, cell lines, or cell strains include, but are not limited to, mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EB1, EB2, EB3, oncolytic or hybridoma-cell lines. In embodiments, the eukaryotic cells are CHO-cell lines. In embodiments, the eukaryotic cell is a CHO cell. In embodiments, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as for example, EBx® cells, EB14, EB24, EB26, EB66, or EBvl3.

In embodiments, the eukaryotic cell is a human cell. In embodiments, the human cell is a stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In embodiments, the human cell is a differentiated form of any of the cells described herein. In embodiments, the eukaryotic cell is a cell derived from any primary cell in culture. In embodiments, the cell is a stem cell or stem cell line.

In embodiments, the eukaryotic cell is a hepatocyte such as a human hepatocyte, animal hepatocyte, or a non-parenchymal cell. For example, the eukaryotic cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57Bl/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In embodiments, the eukaryotic cell is a plant cell. For example, the plant cell can be of a crop plant such as cassava, corn, sorghum, wheat, or rice. The plant cell can be of an algae, tree, or vegetable. The plant cell can be of a monocot or dicot or of a crop or grain plant, a production plant, fruit, or vegetable. For example, the plant cell can be of a tree, e.g., a citrus tree such as orange, grapefruit, or lemon tree; peach or nectarine trees; apple or pear trees; nut trees such as almond or walnut or pistachio trees; nightshade plants, i.e., potatoes; plants of the genus Brassica, plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

### Delivery Particles

In embodiments, the present disclosure provides a delivery particle comprising a Cas9 effector protein as described herein. In embodiments, the present disclosure also provides a delivery particle comprising a system comprising a Cas9 effector protein as described herein.

In embodiments where the delivery particle comprises a system as described herein, the Cas9 effector protein and the guide polynucleotide are in a complex. In embodiments, the complex further comprises a polynucleotide comprising a tracrRNA sequence.

In embodiments, the delivery particle is a lipid-based system, a liposome, a micelle, a microvesicle, an exosome, or a gene gun. In embodiments, the delivery particle comprises a Cas9 effector protein and a guide polynucleotide. In embodiments, the delivery particle comprises a Cas9 effector protein and a guide polynucleotide, wherein the Cas9 effector protein and the guide polynucleotide are in a complex. In embodiments, the delivery particle comprises a polynucleotide encoding a Cas9 effector protein, a polynucleotide encoding a guide polynucleotide, and a polynucleotide comprising a tracrRNA. In embodiments, the delivery particle comprises a Cas9 effector protein, a guide polynucleotide, and a tracrRNA. In embodiments, the delivery particle comprises a polynucleotide encoding one or more Cas9 effector protein, a polynucleotide encoding one or more guide polynucleotides, and a polynucleotide encoding a tracrRNA.

In embodiments, the delivery particle further comprises a lipid, a sugar, a metal or a protein. In embodiments, the delivery particle is a lipid envelope. In embodiments, the delivery particle is a sugar-based particle, for example, GalNAc. In embodiments, the delivery particle is a nanoparticle. Examples of nanoparticles are described herein. Preparation of delivery particles is further described in U.S. Patent Publication Nos. 2011/0293703, 2012/0251560, and 2013/0302401; and U.S. Pat. Nos. 5,543,158, 5,855,913, 5,895,309, 6,007,845, and 8,709,843, each of which is incorporated by reference herein in its entirety.

### Vesicles

In embodiments, the present disclosure provides a vesicle comprising a Cas9 effector protein as described herein. In embodiments, the present disclosure also provides a vesicle comprising a system comprising a Cas9 effector protein as described herein.

In embodiments where the vesicle comprises a system as described herein, the Cas9 effector protein and the guide polynucleotide are in a complex. In embodiments, the complex further comprises a polynucleotide comprising a tracrRNA sequence.

A “vesicle” is a small structure within a cell having a fluid enclosed by a lipid bilayer. Examples of vesicles are provided herein. In embodiments, the vesicle comprises a Cas9 effector protein and a guide polynucleotide. In embodiments, the vesicle comprises a Cas9 effector protein and a guide polynucleotide, wherein the Cas9 effector protein and the guide polynucleotide are in a complex. In embodiments, the vesicle comprises a polynucleotide encoding a Cas9 effector protein, a polynucleotide encoding a guide polynucleotide, and a polynucleotide comprising a tracrRNA. In embodiments, the vesicle comprises a Cas9 effector protein, a guide polynucleotide, and a tracrRNA. In embodiments, the vesicle comprises a polynucleotide encoding one or more Cas9 effector protein, a polynucleotide encoding one or more guide polynucleotides, and a polynucleotide encoding a tracrRNA.

In embodiments, the vesicle is an exosome or a liposome. In embodiments, the Cas9 effector protein is delivered into the cell via an exosome. Exosomes are endogenous nano-vesicles (i.e., having a diameter of about 30 to about 100 nm) that transport RNAs and proteins, and which can deliver RNA to the brain and other target organs. Engineered exosomes for delivery of exogenous biological materials into target organs is described, for example, by Alvarez-Erviti et al., Nature Biotechnology 29: 341 (2011), El-Andaloussi et al., Nature Protocols 7: 2112-2116 (2012), and Wahlgren et al., Nucleic Acids Research 40(17): e130 (2012), each of which is incorporated by reference herein in its entirety.

In embodiments, Cas9 effector protein is delivered into the cell via a liposome. Liposomes are spherical vesicle structures having at least one lipid bilayer and can be used as a vehicle for administration of nutrients and pharmaceutical drugs. Liposomes are often composed of phospholipids, in particular phosphatidylcholine, but also other lipids such as egg phosphatidylethanolamine. Types of liposomes include, but are not limited to, multilamellar vesicle, small unilamellar vesicle, large unilamellar vesicle, and cochleate vesicle. See, e.g., Spuch and Navarro, “Liposomes for Targeted Delivery of Active Agents against Neurodegenerative Diseases (Alzheimer's Disease and Parkinson's Disease), Journal of Drug Delivery 2011, Article ID 469679 (2011). Liposomes for delivery of biological materials such as CRISPR-Cas components are described, for example, by Morrissey et al., Nature Biotechnology 23(8): 1002-1007 (2005), Zimmerman et al., Nature Letters 441: 111-114 (2006), and Li et al., Gene Therapy 19: 775-780 (2012), each of which is incorporated by reference herein in its entirety.

### Viral Vectors

In embodiments, the present disclosure provides a viral vector comprising a Cas9 effector protein as described herein. In embodiments, the present disclosure also provides a viral vector comprising a system comprising a Cas9 effector protein as described herein.

In embodiments where the viral vector comprises a system as described herein, the Cas9 effector protein and the guide polynucleotide are in a complex. In embodiments, the complex further comprises a polynucleotide comprising a tracrRNA sequence.

In embodiments, the viral vector is an adenovirus particle, an adeno-associated virus particle or a herpes simplex virus particle. In embodiments, the viral vector is of an adenovirus, a lentivirus, or an adeno-associated virus. Examples of viral vectors are provided herein. Viral transduction with adeno-associated virus (AAV) and lentiviral vectors (where administration can be local, targeted or systemic) have been used as delivery methods for in vivo gene therapy. In embodiments of the present disclosure, the Cas effector protein is expressed intracellularly by transduced cells.

In embodiments, the viral vector comprises a Cas9 effector protein and a guide polynucleotide. In embodiments, the viral vector comprises a Cas9 effector protein and a guide polynucleotide, wherein the Cas9 effector protein and the guide polynucleotide are in a complex. In embodiments, the viral vector comprises a polynucleotide encoding a Cas9 effector protein, a polynucleotide encoding a guide polynucleotide, and a polynucleotide comprising a tracrRNA. In embodiments, the viral vector comprises a Cas9 effector protein, a guide polynucleotide, and a tracrRNA. In embodiments, the viral vector comprises a polynucleotide encoding one or more Cas9 effector protein, a polynucleotide encoding one or more guide polynucleotides, and a polynucleotide encoding a tracrRNA.

### Methods for Providing Site-Specific Modification of a Target Sequence

In embodiments, the present disclosure provides a method for providing site-specific modification of a target sequence in a eukaryotic cell, the method comprising:
a) introducing into the cell:
   i) a nucleotide encoding a Cas9 effector protein comprising:
   A) a first nuclear localization signal attached to the N-terminus of the Cas9 effector protein; and
   B) a second nuclear localization signal attached to the C-terminus of the Cas9 effector protein; and
   ii) a nucleotide encoding a guide polynucleotide that forms a complex with the Cas9 effector protein and comprises a guide sequence, wherein the guide sequence is capable of hybridizing with a target sequence in host polynucleotide;
b) generating cohesive ends in the host polynucleotide with the Cas9 effector protein and the guide polynucleotide; and
c) ligating
   i) the cohesive ends of (b) together, or
   ii) a 3′ end of a polynucleotide sequence of interest to one cohesive end, and a 5′ end of the polynucleotide sequence to one cohesive end; thereby modifying the target sequence.

A “modification” of a target sequence encompasses single-nucleotide substitutions, multiple-nucleotide substitutions, insertions (i.e., knock-in) and deletions (i.e., knock-out) of a nucleic acid, frameshift mutations, and other nucleic acid modifications.

In embodiments, the modification is a deletion of at least part of the target sequence. A target sequence can be cleaved at two different sites and generate complementary cohesive ends, and the complementary cohesive ends can be re-ligated, thereby removing the sequence portion in between the two sites.

In embodiments, the modification is a mutation of the target sequence. Site-specific mutagenesis in eukaryotic cells is achieved by the use of site-specific nucleases that promote homologous recombination of an exogenous polynucleotide template (also called a “donor polynucleotide” or “donor vector”) containing a mutation of interest. In embodiments, a sequence of interest (SoI) comprises a mutation of interest.

In embodiments, the modification is inserting a sequence of interest (SoI) into the target sequence. The SoI can be introduced as an exogenous polynucleotide template. In embodiments, the exogenous polynucleotide template comprises cohesive ends. In embodiments, the exogenous polynucleotide template comprises cohesive ends complementary to cohesive ends in the target sequence.

The exogenous polynucleotide template can be of any suitable length, such as about or at least about 10, 15, 20, 25, 50, 75, 100, 150, 200, 250, 500 or 1000 or more nucleotides in length. In embodiments, the exogenous polynucleotide template is complementary to a portion of a polynucleotide comprising the target sequence. When optimally aligned, the exogenous polynucleotide template overlaps with one or more nucleotides of a target sequence (e.g., about or at least about 1, 5, 10, 15, 20, 25, 30, 35, 40, 45, 50, 60, 70, 80, 90, or 100 or more nucleotides). In embodiments, when the exogenous polynucleotide template and a polynucleotide comprising the target sequence are optimally aligned, the nearest nucleotide of the exogenous polynucleotide template is within about 1, 5, 10, 15, 20, 25, 50, 75, 100, 200, 300, 400, 500, 100, 1500, 2000, 2500, 5000, 10000 or more nucleotides from the target sequence.

In embodiments, the exogenous polynucleotide is DNA, such as, e.g., a DNA plasmid, a bacterial artificial chromosome (BAC), a yeast artificial chromosome (YAC), a viral vector, a linear piece of single-stranded or double-stranded DNA, an oligonucleotide, a PCR fragment, a naked nucleic acid, or a nucleic acid complexed with a delivery vehicle such as a liposome.

In embodiments, the exogenous polynucleotide is inserted into the target sequence using an endogenous DNA repair pathway of the cell. Endogenous DNA repair pathways include the Non-Homologous End Joining (NHEJ) pathway, Microhomology-Mediated End Joining (MMEJ) pathway, and the Homology-Directed Repair (HDR) pathway. NHEJ, MMEJ, and HDR pathways repair double-stranded DNA breaks. In NHEJ, a homologous template is not required for repairing breaks in the DNA. NHEJ repair can be error-prone, although errors are decreased when the DNA break comprises compatible overhangs. NHEJ and MMEJ are mechanistically distinct DNA repair pathways with different subsets of DNA repair enzymes involved in each of them. Unlike NHEJ, which can be precise as well as error-prone, MMEJ is always error-prone and results in both deletion and insertions at the site under repair. MMEI-associated deletions are due to the micro-homologies (2-10 base pairs) at both sides of a double-strand break. In contrast, HDR requires a homologous template to direct repair, but HDR repairs are typically high-fidelity and less error-prone. In embodiments, the error-prone nature of NHEJ and MMEJ repairs is exploited to introduce non-specific nucleotide substitutions in the target sequence. In embodiments, the Cas9 effector protein cuts the target sequence in a manner that facilitates HDR repair.

During the repair process, an exogenous polynucleotide template comprising the SoI can be introduced into the target sequence. In embodiments, an exogenous polynucleotide template comprising the SoI flanked by an upstream sequence and a downstream sequence is introduced into the cell, wherein the upstream and downstream sequences share sequence similarity with either side of the site of integration in the target sequence. In embodiments, the exogenous polynucleotide comprising the SoI comprises, for example, a mutated gene. In embodiments, the exogenous polynucleotide comprises a sequence endogenous or exogenous to the cell. In embodiments, the SoI comprises polynucleotides encoding a protein, or a non-coding sequence such as, e.g., a microRNA. In embodiments, the SoI is operably linked to a regulatory element. In embodiments, the SoI is a regulatory element. In embodiments, the SoI comprises a resistance cassette, e.g., a gene that confers resistance to an antibiotic. In embodiments, the SoI comprises a mutation of the wild-type target sequence. In embodiments, the SoI disrupts or corrects the target sequence by creating a frameshift mutation or nucleotide substitution. In embodiments, the SoI comprises a marker. Introduction of a marker into a target sequence can make it easy to screen for targeted integrations. In embodiments, the marker is a restriction site, a fluorescent protein, or a selectable marker. In embodiments, the SoI is introduced as a vector comprising the SoI.

The upstream and downstream sequences in the exogenous polynucleotide template are selected to promote homologous recombination between the target sequence and the exogenous polynucleotide. The upstream sequence is a nucleic acid sequence that shares sequence similarity with the sequence upstream of the targeted site for integration (i.e., the target sequence). Similarly, the downstream sequence is a nucleic acid sequence that shares sequence similarity with the sequence downstream of the targeted site for integration. Thus, in embodiments, the exogenous polynucleotide template comprising the SoI is inserted into the target sequence by homologous recombination at the upstream and downstream sequences. In embodiments, the upstream and downstream sequences in the exogenous polynucleotide template have at least 70%, at least 75%, at least 80%, at least 85%, at least 90%, at least 95%, at least 96%, at least 97%, at least 98%, at least 99%, or 100% sequence identity with the upstream and downstream sequences of the targeted genome sequence, respectively. In embodiments, the upstream or downstream sequence has about 20 to 2000 base pairs, or about 50 to 1750 base pairs, or about 100 to 1500 base pairs, or about 200 to 1250 base pairs, or about 300 to 1000 base pairs, or about 400 to about 750 base pairs, or about 500 to 600 base pairs. In embodiments, the upstream or downstream sequence has about 50, about 100, about 250, about 500, about 100, about 1250, about 1500, about 1750, about 2000, about 2250, or about 2500 base pairs.

In embodiments, the modification in the target sequence is inactivation of expression of the target sequence in the cell. For example, upon the binding of a CRISPR complex to the target sequence, the target sequence is inactivated such that the sequence is not transcribed, the coded protein is not produced, or the sequence does not function as the wild-type sequence does. For example, a protein or microRNA coding sequence may be inactivated such that the protein is not produced.

In embodiments, a regulatory sequence can be inactivated such that it no longer functions as a regulatory sequence. Examples of a regulatory sequence include a promoter, a transcription terminator, an enhancer, and other regulatory elements described herein. The inactivated target sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). In embodiments, the inactivation of a target sequence results in “knockout” of the target sequence.

In embodiments of the method, the first nuclear localization signal is a monopartite nuclear localization signal. In embodiments, the first nuclear localization signal is a bipartite nuclear localization signal. In embodiments, the second nuclear localization signal is a monopartite nuclear localization signal. In embodiments, the second nuclear localization signal is a bipartite nuclear localization signal.

In embodiments of the method, the first and second nuclear localization signals can both be monopartite, both be bipartite or can be a mixture of monopartite and bipartite. In embodiments, the first nuclear localization signal is a bipartite nuclear localization signal and the second nuclear localization signal is a monopartite nuclear localization signal. In embodiments, the first nuclear localization signal is a monopartite nuclear localization signal and the second nuclear localization signal is a bipartite nuclear localization signal. In embodiments, the first nuclear localization signal is a monopartite nuclear localization signal and the second nuclear localization signal is a monopartite nuclear localization signal. In embodiments, the first nuclear localization signal is a bipartite nuclear localization signal and the second nuclear localization signal is a bipartite nuclear localization signal.

In embodiments of the method, the monopartite nuclear localization signal is a monopartite nuclear localization signal known in the art. In embodiments, the monopartite nuclear localization signal is one of the monopartite nuclear localization signals listed in Table 1 above (SEQ ID NOs: 1-6), or combinations thereof.

In embodiments of the method, the bipartite nuclear localization signal is a bipartite nuclear localization signal known in the art. In embodiments, the bipartite nuclear localization signal is a classical bipartite nuclear localization signal. In embodiments, the bipartite nuclear localization signal is one of the bipartite nuclear localization signals listed in Table 2 above (SEQ ID NOs: 7-9), or combinations thereof.

In embodiments of the method, the first nuclear localization signal is classic bipartite nuclear localization signal (SEQ ID NO: 7) and the second nuclear localization signal is SV40 Large T-Antigen nuclear localization signal (SEQ ID NO: 1).

In embodiments of the method, the first nuclear localization signal is directly attached to the Cas9 effector protein. In embodiments, the first nuclear localization signal is attached to the Cas9 effector protein via a linker. In embodiments, the second nuclear localization signal is directly attached to the Cas9 effector protein. In embodiments, the second nuclear localization signal is attached to the Cas9 effector protein via a linker.

In embodiments where a linker is used, the linker is a peptide linker having from 2 to 30 residues. In embodiments, linker is a peptide linker having from 2 to 20 residues. In embodiments, linker is a peptide linker having from 2 to 15 residues. In embodiments, linker is a peptide linker having from 2 to 10 residues. In embodiments, linker is a peptide linker having from 2 to 5 residues. In embodiments, the linker is a substituted or unsubstituted C₂-C₂₀ alkyl, alkene or alkynyl chain.

In embodiments of the method, the Cas9 effector protein comprises more than one copy of a nuclear localization signal on its N-terminus. In embodiments, the Cas9 effector protein comprises more than one type of nuclear localization signal on its N-terminus. In embodiments, the Cas9 effector protein comprises more than one copy of a nuclear localization signal on its C-terminus. In embodiments, the Cas9 effector protein comprises more than one type of nuclear localization signal on its C-terminus.

In embodiments of the method, the protein comprises two copies of the first nuclear localization signal. In embodiments, the protein comprises three copies of the first nuclear localization signal. In embodiments, the protein comprises two copies of the second nuclear localization signal. In embodiments, the protein comprises three copies of the second nuclear localization signal.

In embodiments of the method, the Cas9 portion of the Cas9 protein comprising a first and a second nuclear localization signal can be derived from any Cas9 effector domain known in the art. In embodiments, the Cas9 effector protein is derived from a bacterial species having a Type II-B CRISPR system. Examples of suitable Type II-B Cas9 proteins are described above. In embodiments, the Cas9 portion comprises a domain that matches the TIGR03031 protein family with an E-value cut-off of 1E-5. In embodiments, the site-specific nuclease comprises a domain that matches the TIGR03031 protein family with an E-value cut-off of 1E-10.

In embodiments of the method, the Cas9 effector protein comprises a polypeptide sequence having at least 95% identity to any one of SEQ ID NOs: 10-97 as shown in FIG. 1 and FIG. 2. In embodiments, the Cas9 effector protein comprises a polypeptide sequence having at least 90% identity to any one of SEQ ID NOs: 10-97 as shown in FIG. 1 and FIG. 2. In embodiments, the Cas9 effector protein comprises a polypeptide sequence having at least 98% identity to any one of SEQ ID NOs: 10-97 as shown in FIG. 1 and FIG. 2. In embodiments, the Cas9 effector protein comprises a polypeptide sequence having at least 99% identity to any one of SEQ ID NOs: 10-97 as shown in FIG. 1 and FIG. 2. In embodiments, the Cas9 effector protein comprises a polypeptide selected from one of SEQ ID NOs: 10-97 as shown in FIG. 1 and FIG. 2.

In embodiments of the method, the Cas9 effector protein comprises a polypeptide sequence having at least 95% identity to SEQ ID NO: 71. In embodiments, the Cas9 effector protein comprises a polypeptide sequence having at least 90% identity to SEQ ID NO: 71. In embodiments, the Cas9 effector protein comprises a polypeptide sequence having at least 98% identity to SEQ ID NO: 71. In embodiments, the Cas9 effector protein comprises a polypeptide sequence having at least 99% identity to SEQ ID NO: 71. In embodiments, the Cas9 effector protein comprises SEQ ID NO: 71.

In embodiments of the method, the Cas9 effector protein comprises a polypeptide sequence at least 90% identical to SEQ ID NO: 98. In embodiments, the Cas9 effector protein comprises a polypeptide sequence at least 95% identical to SEQ ID NO: 98. In embodiments, the Cas9 effector protein comprises a polypeptide sequence at least 98% identical to SEQ ID NO: 98. In embodiments, the Cas9 effector protein comprises a polypeptide sequence at least 99% identical to SEQ ID NO: 98. In embodiments, the Cas9 effector protein comprises SEQ ID NO: 98.

In other embodiments of the method, the Cas9 portion of the Cas9 effector protein comprises a dCas9, i.e., a deactivated or “dead” Cas9 lacking DNA double strand break activity. In embodiments, the dCas9 can be fused with other active domains, such as transcriptional regulators, epigenetic regulator proteins or fluorescent proteins as described elsewhere herein. In embodiments where a dCas9 is fused to another active domain, the nuclear localization signals described herein are present at the N-terminus and C-terminus of the overall Cas9 effector protein construct.

In other embodiments of the method, the Cas9 portion of the Cas9 effector protein comprises a Cas9 nickase, i.e., a Cas9 protein that only cleaves one strand of the DNA double strand. In embodiments, the Cas9 nickase can be fused with other active domains, such as transcriptional regulators, epigenetic regulator proteins or fluorescent proteins as described elsewhere herein. In embodiments where a Cas9 nickase is fused to another active domain, the nuclear localization signals described herein are present at the N-terminus and C-terminus of the overall Cas9 effector protein construct.

In embodiments, the method comprises use of a guide polynucleotide as described herein. In embodiments of the method, the guide polynucleotide is an RNA.

In embodiments of any of the above systems, the guide sequence is from 19 to 30 bases in length. In embodiments, the guide sequence is from 19 to 25 bases in length. In embodiments, the guide sequence is from 21 to 26 bases in length.

In embodiments of any of the above systems, the guide polynucleotide further comprises a tracrRNA sequence as described herein. In embodiments of the systems disclosed herein, the Cas9 effector protein, guide polynucleotide, and tracrRNA are capable of forming a complex.

In embodiments of the method, the Cas9 effector protein generates cohesive ends. In embodiments, the cohesive ends generated by the Cas9 effector protein comprise a 5′ overhang. In embodiments, the cohesive ends generated by the Cas9 effector protein comprise a 3′ overhang. In embodiments, the cohesive ends comprise a single-stranded polynucleotide overhang of 1 to 10 nucleotides. In embodiments, the cohesive ends comprise a single-stranded polynucleotide overhang of 2 to 6 nucleotides. In embodiments, the cohesive ends comprise a single-stranded polynucleotide overhang of 3 to 5 nucleotides.

In embodiments of the method, the eukaryotic cell is an animal or human cell. In embodiments, the eukaryotic cell is an animal cell as described herein. In embodiments, the eukaryotic cell is a human cell. In embodiments, the eukaryotic cell is a human cell as described herein. In embodiments, the eukaryotic cell is a plant cell. In embodiments, the eukaryotic cell is a plant cell as described herein.

In embodiments of the method, the modification is deletion of at least part of the target sequence. In embodiments, the modification is mutation of the target sequence. In embodiments, the modification is inserting a sequence of interest into the target sequence. In embodiments, the modification is a modification as described herein.

In embodiments of the method, the modification is provided with reduced off-target effects. In embodiments of the method, the modification is provided with reduced off-target effects compared to off-target effects provided with S. pyogenes Cas9 (SpCas9).

The present disclosure also provides a method for providing site-specific modification of a target sequence in a eukaryotic cell with reduced off-target effects, the method comprising:
a) introducing into the cell: i) a nucleotide encoding a Cas9 effector protein comprising: A) a first nuclear localization signal attached to the N-terminus of the Cas9 effector protein; and B) a second nuclear localization signal attached to the C-terminus of the Cas9 effector protein; and ii) a nucleotide encoding a guide polynucleotide that forms a complex with the Cas9 effector protein and comprises a guide sequence, wherein the guide sequence is capable of hybridizing with a target sequence in host polynucleotide;
b) generating cohesive ends in the host polynucleotide with the Cas9 effector protein and the guide polynucleotide; and
c) ligating i) the cohesive ends of (b) together, or ii) a 3′ end of a polynucleotide sequence of interest to one cohesive end, and a 5′ end of the polynucleotide sequence to one cohesive end; thereby modifying the target sequence with reduced off-target effects.

In embodiments of the method, the modification is provided with reduced off-target effects compared to off-target effects provided with S. pyogenes Cas9 (SpCas9). In embodiments of the method, the modification is provided with reduced off-target effects compared to off-target effects provided with wild-type S. pyogenes Cas9 (SpCas9).

### Methods for Reducing Degradation of a Cas9 Effector Protein

In embodiments, the present disclosure provides a method for reducing degradation of Cas9 effector protein in a cell comprising:
a) attaching a first nuclear localization signal to the N-terminus of the Cas9 effector protein; and
b) attaching a second nuclear localization signal to the C-terminus of the Cas9 effector protein.

In embodiments, the attaching can be performed as described herein. In embodiments, nucleic acid sequences encoding for the nuclear localization signals are placed upstream and downstream from a nucleic acid sequence encoding the Cas9 effector protein using standard molecular biology methods such as restriction enzyme digestion and ligation, so that a nucleic acid is formed that encodes the Cas9 effector protein comprising a nuclear localization signal on its N-terminus and C-terminus. This nucleic acid can then be subsequently expressed in a cell, e.g., a eukaryotic cell. In other embodiments, the Cas9 effector protein comprising nuclear localization signals on its N-terminus and C-terminus is fully or partially synthesized using solid-phase protein synthesis methods.

In embodiments of the method, the first nuclear localization signal is a monopartite nuclear localization signal. In embodiments, the first nuclear localization signal is a bipartite nuclear localization signal. In embodiments, the second nuclear localization signal is a monopartite nuclear localization signal. In embodiments, the second nuclear localization signal is a bipartite nuclear localization signal.

In embodiments of the method, the first and second nuclear localization signals can both be monopartite, both be bipartite or can be a mixture of monopartite and bipartite. In embodiments, the first nuclear localization signal is a bipartite nuclear localization signal and the second nuclear localization signal is a monopartite nuclear localization signal. In embodiments, the first nuclear localization signal is a monopartite nuclear localization signal and the second nuclear localization signal is a bipartite nuclear localization signal.

In embodiments, the first nuclear localization signal is a monopartite nuclear localization signal and the second nuclear localization signal is a monopartite nuclear localization signal. In embodiments, the first nuclear localization signal is a bipartite nuclear localization signal and the second nuclear localization signal is a bipartite nuclear localization signal.

In embodiments of the method, the monopartite nuclear localization signal is a monopartite nuclear localization signal known in the art. In embodiments, the monopartite nuclear localization signal is one of the monopartite nuclear localization signals listed in Table 1 above (SEQ ID NOs: 1-6), or combinations thereof.

In embodiments of the method, the bipartite nuclear localization signal is a bipartite nuclear localization signal known in the art. In embodiments, the bipartite nuclear localization signal is a classical bipartite nuclear localization signal. In embodiments, the bipartite nuclear localization signal is one of the bipartite nuclear localization signals listed in Table 2 above (SEQ ID NOs: 7-9), or combinations thereof.

In embodiments of the method, the first nuclear localization signal is classic bipartite nuclear localization signal (SEQ ID NO: 7) and the second nuclear localization signal is SV40 Large T-Antigen nuclear localization signal (SEQ ID NO: 1).

In embodiments of the method, the first nuclear localization signal is directly attached to the Cas9 effector protein. In embodiments, the first nuclear localization signal is attached to the Cas9 effector protein via a linker. In embodiments, the second nuclear localization signal is directly attached to the Cas9 effector protein. In embodiments, the second nuclear localization signal is attached to the Cas9 effector protein via a linker.

In embodiments where a linker is used, the linker is a peptide linker having from 2 to 30 residues. In embodiments, linker is a peptide linker having from 2 to 20 residues. In embodiments, linker is a peptide linker having from 2 to 15 residues. In embodiments, linker is a peptide linker having from 2 to 10 residues. In embodiments, linker is a peptide linker having from 2 to 5 residues. In embodiments, the linker is a substituted or unsubstituted C₂-C₂₀ alkyl, alkene or alkynyl chain.

In embodiments of the method, the Cas9 effector protein comprises more than one copy of a nuclear localization signal on its N-terminus. In embodiments, the Cas9 effector protein comprises more than one type of nuclear localization signal on its N-terminus. In embodiments, the Cas9 effector protein comprises more than one copy of a nuclear localization signal on its C-terminus. In embodiments, the Cas9 effector protein comprises more than one type of nuclear localization signal on its C-terminus.

In embodiments of the method, the protein comprises two copies of the first nuclear localization signal. In embodiments, the protein comprises three copies of the first nuclear localization signal. In embodiments, the protein comprises two copies of the second nuclear localization signal. In embodiments, the protein comprises three copies of the second nuclear localization signal.

In embodiments of the method, the Cas9 portion of the Cas9 protein comprising a first and a second nuclear localization signal can be derived from any Cas9 effector domain known in the art. In embodiments, the Cas9 effector protein is derived from a bacterial species having a Type II-B CRISPR system. Examples of suitable Type II-B Cas9 proteins are described above. In embodiments, the Cas9 portion comprises a domain that matches the TIGR03031 protein family with an E-value cut-off of 1E-5. In embodiments, the site-specific nuclease comprises a domain that matches the TIGR03031 protein family with an E-value cut-off of 1E-10.

In embodiments of the method, the Cas9 effector protein comprises a polypeptide sequence having at least 95% identity to any one of SEQ ID NOs: 10-97 as shown in FIG. 1 and FIG. 2. In embodiments, the Cas9 effector protein comprises a polypeptide sequence having at least 90% identity to any one of SEQ ID NOs: 10-97 as shown in FIG. 1 and FIG. 2. In embodiments, the Cas9 effector protein comprises a polypeptide sequence having at least 98% identity to any one of SEQ ID NOs: 10-97 as shown in FIG. 1 and FIG. 2. In embodiments, the Cas9 effector protein comprises a polypeptide sequence having at least 99% identity to any one of SEQ ID NOs: 10-97 as shown in FIG. 1 and FIG. 2. In embodiments, the Cas9 effector protein comprises a polypeptide selected from one of SEQ ID NOs: 10-97 as shown in FIG. 1 and FIG. 2.

In embodiments of the method, the Cas9 effector protein comprises a polypeptide sequence having at least 95% identity to SEQ ID NO: 71. In embodiments, the Cas9 effector protein comprises a polypeptide sequence having at least 90% identity to SEQ ID NO: 71. In embodiments, the Cas9 effector protein comprises a polypeptide sequence having at least 98% identity to SEQ ID NO: 71. In embodiments, the Cas9 effector protein comprises a polypeptide sequence having at least 99% identity to SEQ ID NO: 71. In embodiments, the Cas9 effector protein comprises SEQ ID NO: 71.

In embodiments of the method, the Cas9 effector protein comprises a polypeptide sequence at least 90% identical to SEQ ID NO: 98. In embodiments, the Cas9 effector protein comprises a polypeptide sequence at least 95% identical to SEQ ID NO: 98. In embodiments, the Cas9 effector protein comprises a polypeptide sequence at least 98% identical to SEQ ID NO: 98. In embodiments, the Cas9 effector protein comprises a polypeptide sequence at least 99% identical to SEQ ID NO: 98. In embodiments, the Cas9 effector protein comprises SEQ ID NO: 98.

All references cited herein, including patents, patent applications, papers, textbooks and the like, and the references cited therein, to the extent that they are not already, are hereby incorporated herein by reference in their entirety.

### Examples

### Example 1—Cas9 Is a Substrate for Lysosomal Degradation

Cas9 protein from the sequence gut metagenome MH0245 (MHCas9)—as described in WO2019099943, which is hereby incorporated by reference herein—was cloned into a plasmid encoding three copies of SV40 monopartite nuclear localization signal (NLS; SEQ ID NO: 1) to form 3×SV40-MHCas9, a Cas9 protein with three SV40 NLS attached to its N-terminus.

The plasmid was transfected into HEK293T cells. Cells were cultured in DMEM+10% FBS medium for 24 hours before addition to the cell culture of either: 1) proteasome inhibitor MG132 at a concentration of 5 μM; 2) lysosomal vATPase inhibitor bafilomycin A1 at a concentration of 20 nM; or 3) nuclear export inhibitor leptomycin B at a concentration of 10 nM. Untreated cells were used as a control. The cells were harvested followed by total protein extraction.

HEK293T cells were seeded at a density of 25,000 cells per well on a 96-well plate the day prior to transfection. 20 hours following seeding, the cells were transfected with the plasmids described above. 48 hours after transfection, 100 μL of media was added to the cells. Cells were harvested 60 hours following transfection.

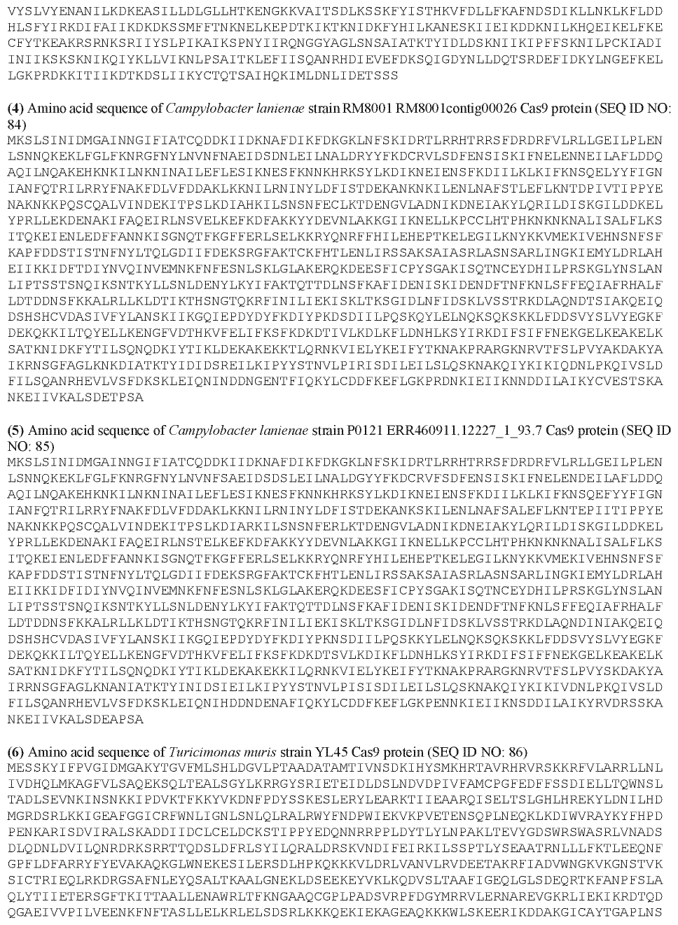

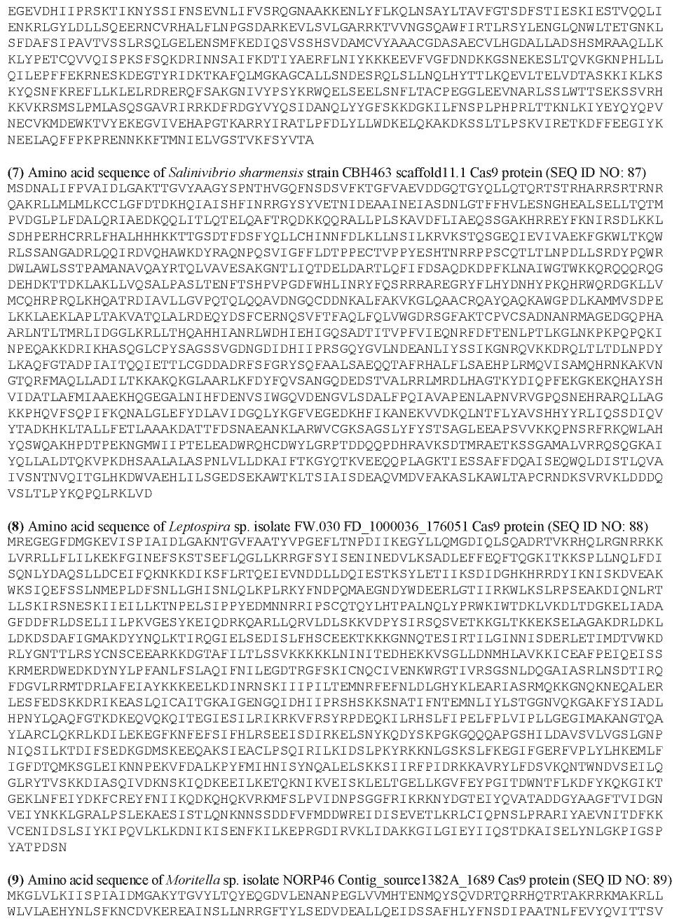

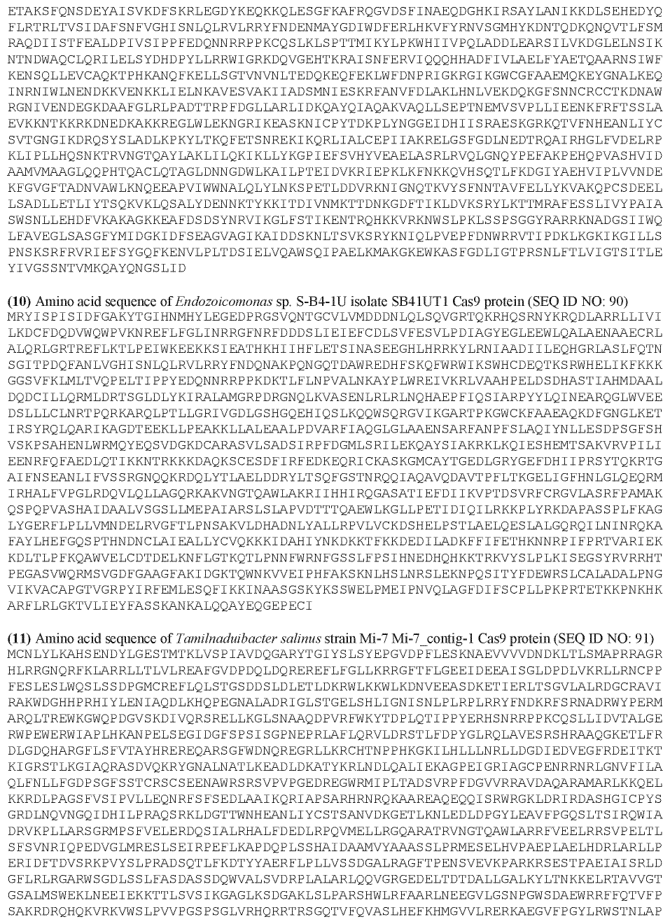

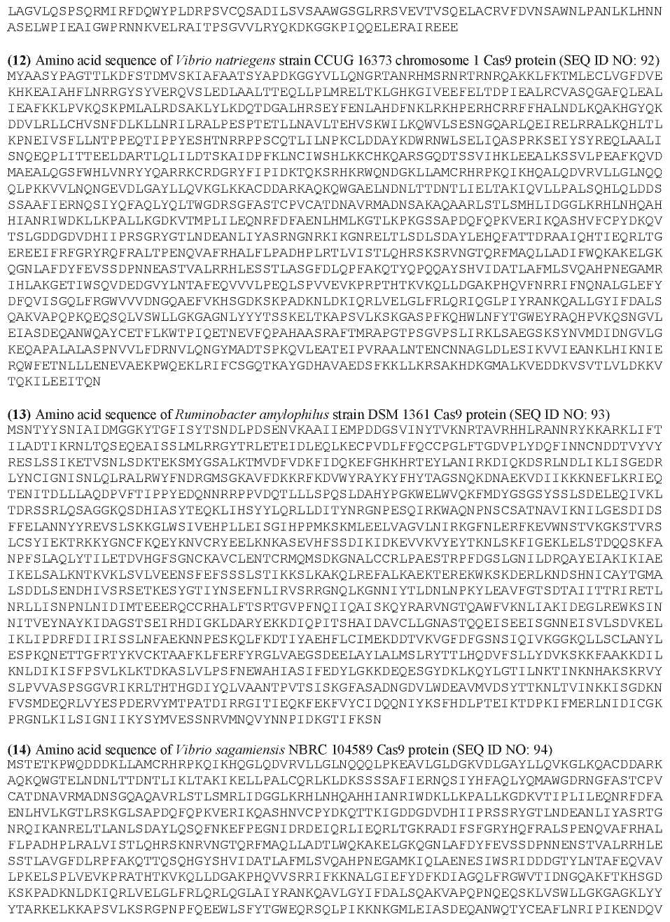

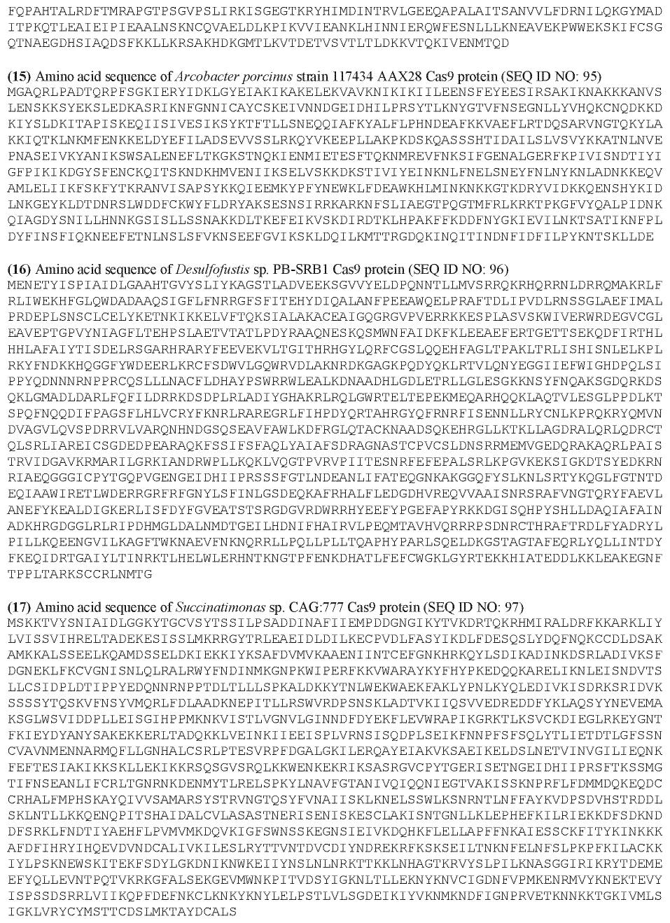

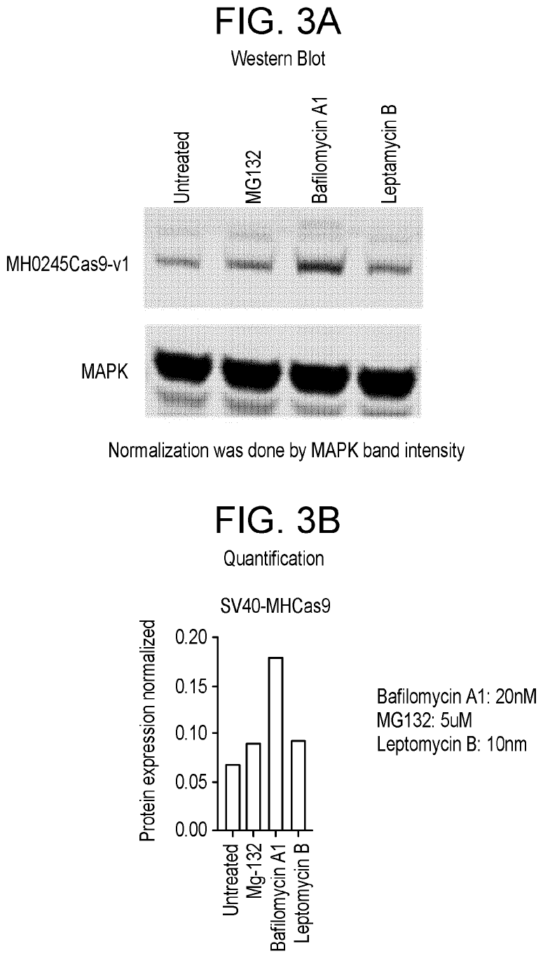

After harvest, western blots were used to analyze total levels of 3×SV40-MHCas9 compared to blots of mitogen-activated protein kinase (MAPK) for normalization of band intensity. The blots are shown in FIG. 3A. The western blots were quantified and normalized protein expression was plotted as shown in FIG. 3B.

As can be seen in FIG. 3B, blocking lysosomal function with bafilomycin A1 lead to increased MHCas9 levels, suggesting that MHCas9 was being degraded in the lysosome.

### Example 2—Addition of NLS to Cas9 Prevents Degradation

MHCas9 as described in Example 1 was cloned into plasmids encoding nuclear localization signals to form four different Cas9 effector protein constructs: 1) 3×SV40-MHCas9 as described in Example 1; 2) an MHCas9 having a single SV40 NLS at the C-terminus (MHCas9-NLSSV40); 3) an MHCas9 having three SV40 NLS at the N-terminus and a single SV40 NLS at the C-terminus (3XNLSSV40-MHCas9-NLSSV40); and 4) an MHCas9 having a single bipartite NLS (SEQ ID NO: 7) at the N-terminus and a single SV40 NLS at the C-terminus (bpNLS-MHCas9-SLSSV40). The plasmids expressed green fluorescent protein (GFP) which was detected for normalization of transfection.

HEK293T cells were transfected and grown as described in Example 1 but were not treated with any inhibitors. Cells were harvested and western blots performed, with tubulin detected as a gel loading control and GFP used for normalization of transfection amounts.

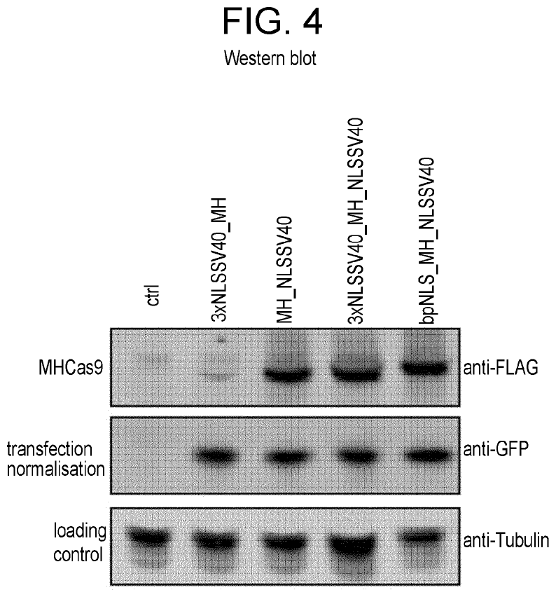

The blots are shown in FIG. 4. As can be seen, the additional of a NLS on the C-terminus of Cas9 increases the stability of the protein in vivo. The bpNLS-MHCas9-SLSSV40 protein was chosen for further study and named SpOT-ON.

### Example 3—Cas9 Constructs With NLS on Both Terminals Avoid Lysosomal Degradation

To test the effect of NLS on other Cas9 proteins, S. pyogenes Cas9 (SpCas9) was cloned into the same vector as construct 4 in Example 2 to form a bpNLS-SpCas9-NLSSV40 construct.

Cells expressing SpOT-ON and bpNLS-SpCas9-NLSSV40 were either left untreated or grown in the presence of the inhibitors MG132, bafilomycin A1 of leptomycin at the same concentrations used in Example 1. Cells were harvested and western blots were performed using MAPK for normalization of band intensity as described in Example 1. Blots for SpOT-ON are shown in FIG. 5A and blots for bpNLS-SpCas9-NLSSV40 are shown in FIG. 5B.

As can be seen in the blots, similar levels of protein are detected regardless of whether the sample is treated with an inhibitor or treated with an inhibitor that did not significantly slow degradation in Example 1 (MG132 and leptomycin B). The enhanced nuclear important provided by the additional NLS signals likely prevents protein degradation in the cytoplasm. Further, the additional of NLS signals at the N- and C-termini leads to enhanced stability for both MHCas9 and SPCas9, suggesting that this technique should be generally applicable to enhance stability of all types of Cas9 proteins.

### Example 4—SpOT-ON Has Similar DNA Cleavage Activity to Unmodified Cas9

The DNA cleavage activity of SpOT-ON was compared to Cas9 lacking a NLS and was found to be similar.

Cleavage activity of SpOT-On and Streptococcus pyogenes Cas9 protein (SpyCas9) were measured in vitro. Cas9 ribonucleoproteins (RNPs) targeting a 20 nt protospacer were mixed with fluorescently labelled target DNA and loading control lacking protospacer adjacent motifs (PAMs). Reactions were incubated at 37 C, aliquots were taken at different time-points, quenched and resolved using capillary electrophoresis. The fraction of DNA digested was quantified, normalized to the loading control and zero timepoint, and then plotted against time. Both enzymes digested the target DNA to the same extent. Analysis of the data (not shown) determined a rate constant (k) for: SpyCas9, k=0.224 and SpOT-ON, k=0.004.

The results showed that SpOT-On Cas9 was capable of digesting targeted DNA to the same extent as SpyCas9 in vitro. However, as seen in the different rate constants, this cleavage happens slower with SpOT-On Cas9 than with SpyCas9.

### Example 5—SpOT-ON Has Similar Editing Activity to Unmodified Cas9

The gene editing activity of SpOT-ON was compared to Cas9 lacking a NLS and was found to be similar.

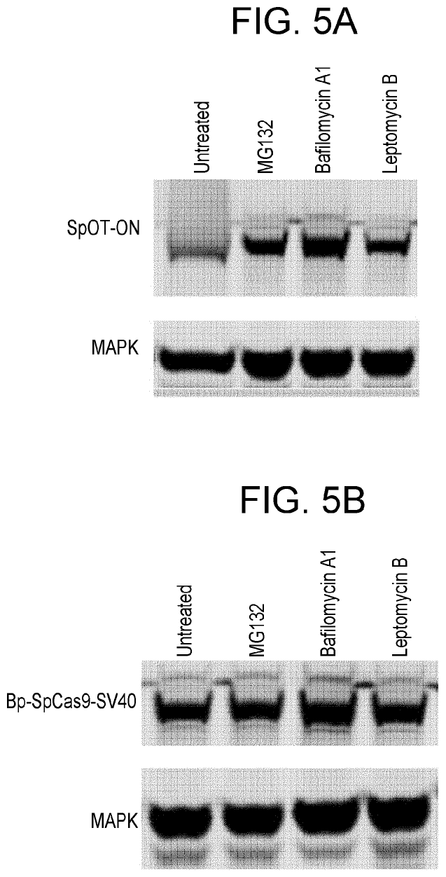

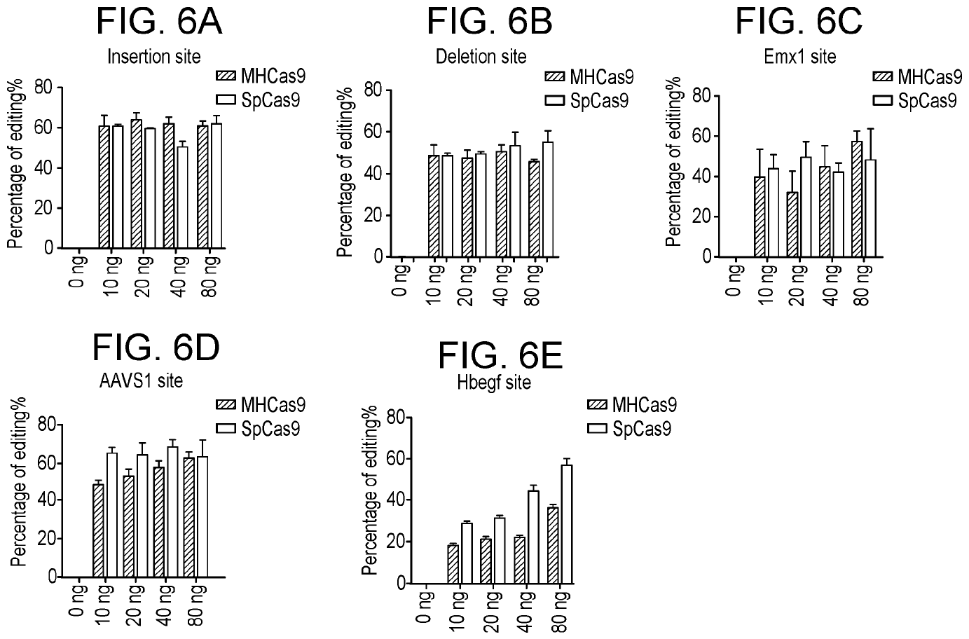

Gene editing activity was compared for SpOT-ON and SpCas9. HEK293T cells were transfected by expression vectors expressing the Cas9 variant and the guideRNA for HEK3, HEK4, EMX1 and FANCF. CD34 was used as the insertion site and STAT1 was used as the deletion site. Cells were cultured for 72 hours and then lysed to obtain DNA. Deep amplicon sequencing was performed to evaluate editing that had occurred. As can be seen in FIG. 6, editing efficiency was similar for SpOT-ON and SpCas9.

### Example 6—Determining Optimal Protospacer Length for SpOT-ON

It was hypothesized that the slow cleavage of DNA by SpOT-On Cas9 seen in Example 4 was caused by suboptimal sgRNA design, in particular protospacer length. The in vitro cleavage experiment of Example 4 was repeated for SpOT-On Cas9 RNPs formed with a series of sgRNAs with varying protospacer targeting sequence length targeting the same sequence.

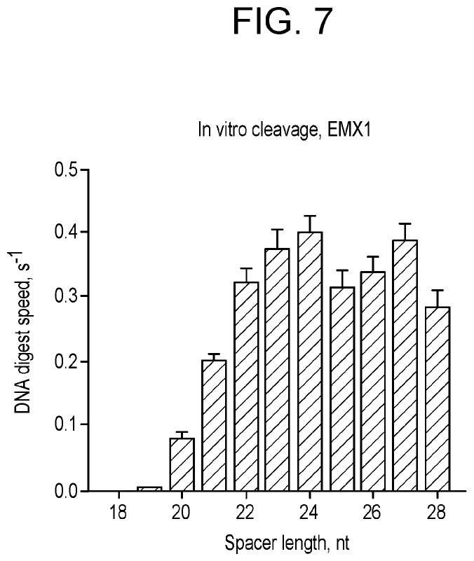

In order to optimize reaction efficiency, cleavage activities for guide RNAs having varying spacer lengths were determined using the methods described in Example 4 and plotted. The bars represent computed speed constant and error bars represent standard error of fitting. Results are shown in FIG. 7.

As can be seen in FIG. 7, SpOT-On Cas9 targeting shorter protospacers (18-20 nt) is less efficient in DNA cleavage. However, RNPs with longer guides digest DNA 10-50 times faster, suggesting that an at least 21 nucleotide target sequence is required for optimal activity of SpOT-On Cas9.

Further studies were performed to determine the optimal protospacer length in vivo. Cleavage activity was tested in vivo at two different target sites: EMX1 and CD34.

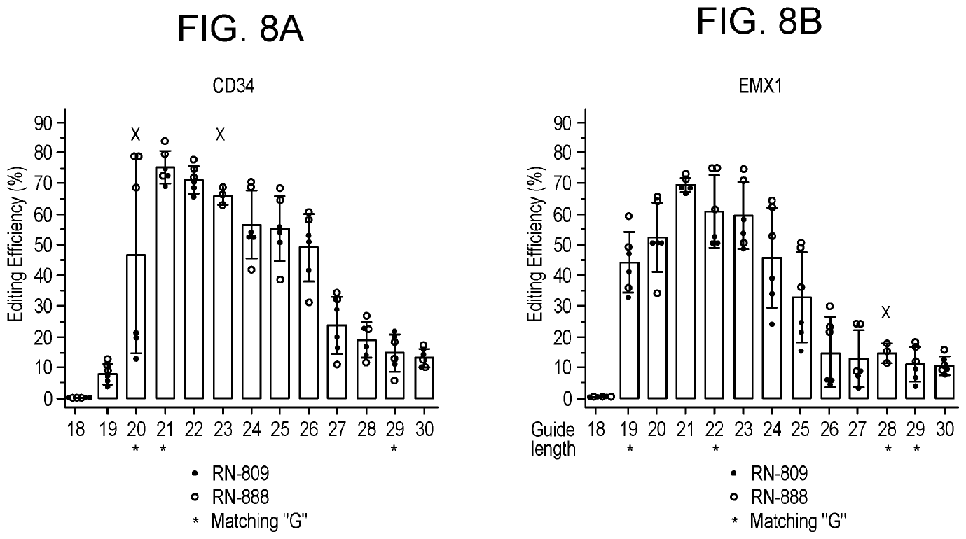

HEK293T cells were seeded at a density of 25,000 cells per well on a 96-well plate the day prior to transfection. 20 hours following seeding, the cells were transfected with the plasmids described above. 48 hours after transfection, 100 μL of media was added to the cells. Cells were harvested 60 hours following transfection using QuickExtract DNA extraction solution (Lucigen). Deep targeted amplicon sequencing was performed. The bar-graphs shown in FIG. 8 show the mean percentage of mutated reads in mapped reads. Number of replicates n=3 (cells were separated into three stocks, then transfected and analyzed separately).

As can be seen in FIG. 8, optimal protospacer length for SpOT-ON is between 19-23 nucleotides, with 21 nucleotides showing peak activity.

### Example 7—SpOT-ON Shows Reduced Off-Target DNA Editing

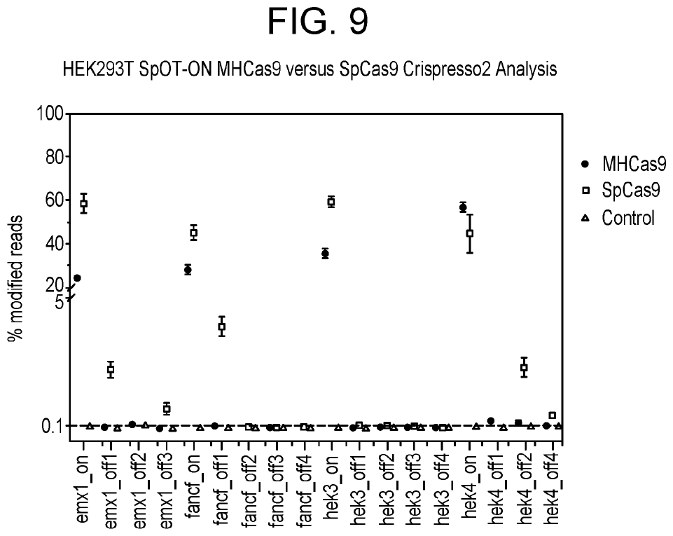

Gene editing activity was compared for SpOT-ON and SpCas9 using methods similar to those described in Example 5. HEK293T cells were transfected by expression vectors expressing the Cas9 variant and the guideRNA for HEK3, HEK4, EMX1 and FANCF. Cells were cultured for 72 hours and then lysed to obtain DNA. Deep amplicon sequencing was performed to evaluate editing that had occurred. Analysis of off-target editing was performed using Crispresso2 pooled analysis. The 14 off-target sites analyzed were those determined by Tsai et al. (Nat Biotechnol. 2015 February; 33(2): 187-197). A plot of the off-target analysis is shown in FIG. 9.

As can be seen in FIG. 9, SpOT-ON showed a greatly reduced percentage of editing at the off-target sites than SpCas9, showing that SpOT-ON is better at discriminating on and off-target sequences.

### Example 8—Analysis of Off-Target DNA Editing

Further studies were performed to investigate how mismatches in the substrate DNA affect the kinetics of DNA cleavage. DNA substrates carrying single base pair substitutions in the target sequence were generated to study specificity of SpyCas9 and SpOT-ON.

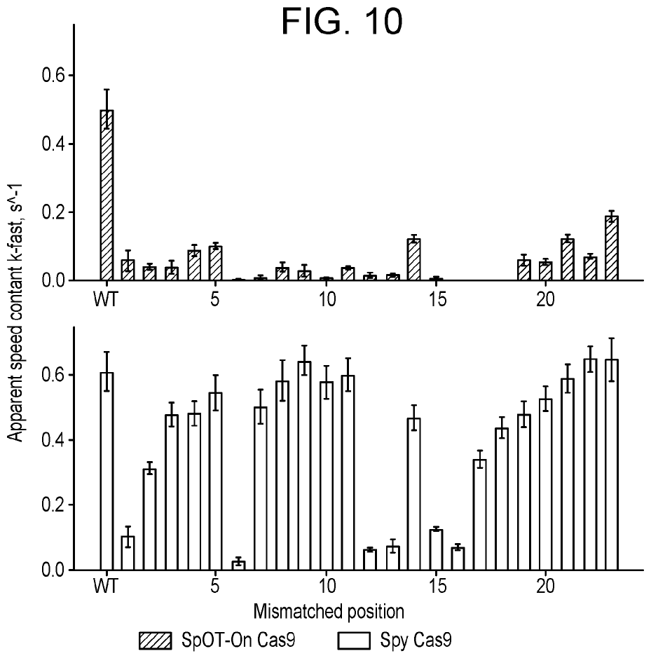

Activity of Cas9 enzymes was measured for perfectly matched and mismatched DNA substrates at positions 1, 2 and 3 from PAM. The experiments were performed as described in the above Examples with optimal guides. Cleavage speed constants for each DNA substrate were calculated and are plotted in FIG. 10.

As shown in FIG. 10, Cas9 enzymes digest mismatched DNA substrates slower than perfectly matched ones. A mismatch immediately adjacent to the PAM results in a dramatic reduction in cleavage speeds for both SpyCas9 and SpOT-On Cas9. More distal mismatches slow down Spy Cas9 activity only marginally whereas SpOT-On Cas9 was inhibited at least 10 times. These data suggests that SpOT-On Cas9 is a more specific enzyme than Spy Cas9 in vitro, potentially explaining low off-target genome editing activity of SpOT-On Cas9 in vivo.

### Example 9—Mismatch Tolerance In Vivo

The in vivo mismatch tolerance of SpOT-ON, mismatch editing of EMX1 was tested with a 23 nucleotide guide RNA in HEK293T cells.

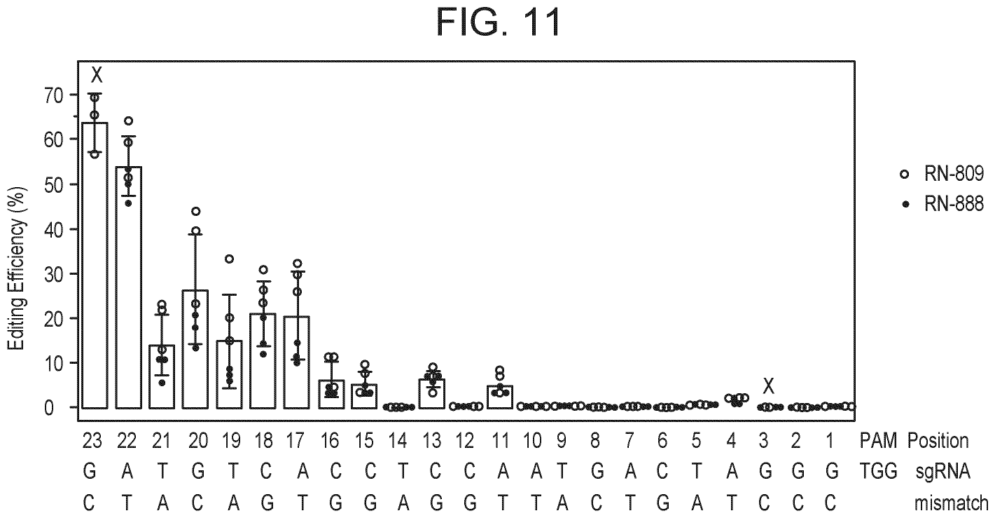

HEK293T cells were seeded at a density of 25,000 cells per well on a 96-well plate the day prior to transfection. 20 hours following seeding, the cells were transfected with the plasmids described above. 48 hours after transfection, 100 μL of media was added to the cells. Cells were harvested 60 hours following transfection using QuickExtract DNA extraction solution (Lucigen). Deep targeted amplicon sequencing was performed. Bar-graphs in FIG. 11 show the mean percentage of mutated reads in mapped reads. Number of replicates n=3 (cells were separated into three stocks, then transfected and analyzed separately).

As can be seen from FIG. 11, mismatches at position 1-10 (with 1 closest to the PAM) are not tolerated and result in no or very low editing (>0.7%). Mismatches between position 11-21 showed medium editing efficiency of up to 20%. A mismatch at position 22 resulted in similar editing efficiency than the sgRNA without mismatch (˜55%).

### Example 10—DNA Editing and Analysis of Cut Site

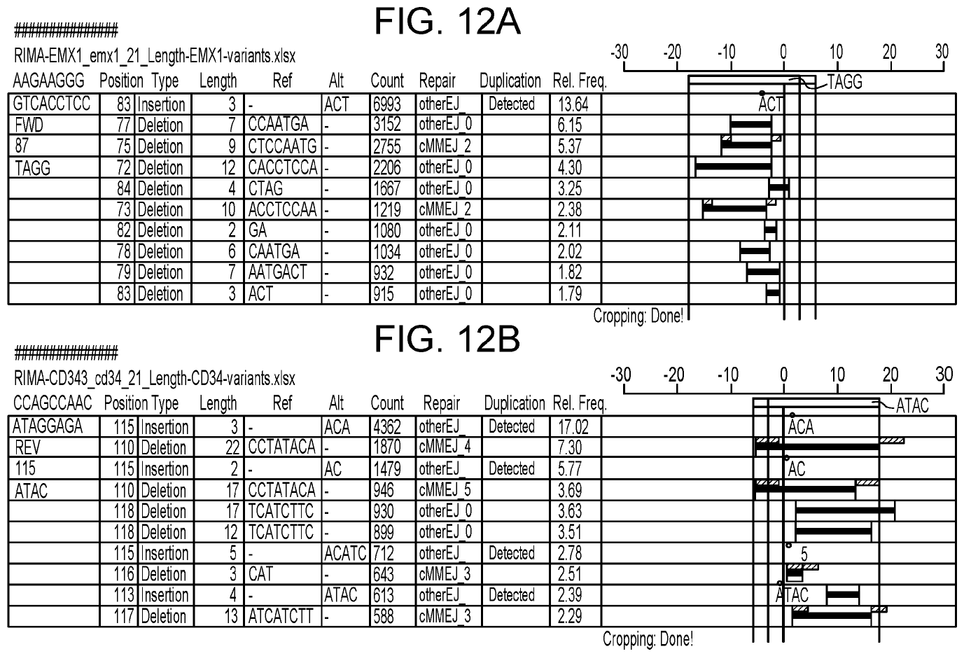

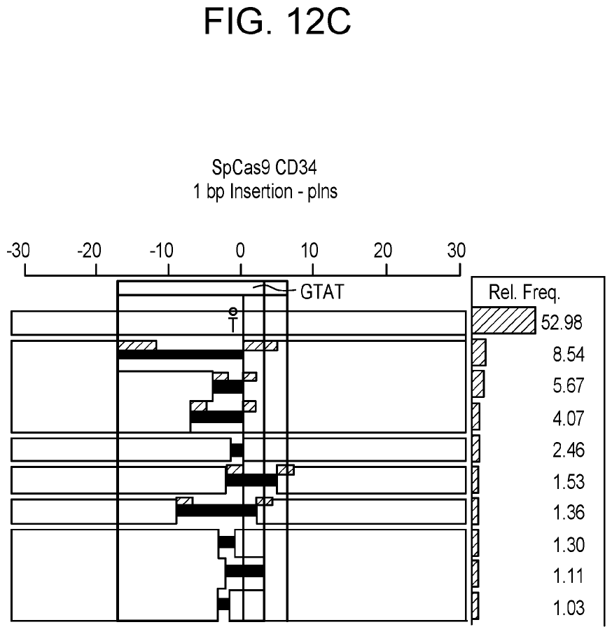

DNA editing at the EMX and CD34 loci was further analyzed for qualitative assessment of the DNA repair outcome. Cells were seeded and grown as described in Example 9. NGS results of Amplicon-sequencing analyzed using RIMA are shown in FIG. 12A for EMX1, FIG. 12B for CD34 and FIG. 12C for a CD34 control with SpCas9.

These results demonstrated that SpOT-ON cuts DNA to generate a 3 nucleotide overhang in HEK293T cells.

### Example 11—Knock-In Experiments

Experiments were performed to evaluate the efficiency of directional non-homologous end joining (NHEJ) mediated knock-in of oligos with blunt ends or different overhangs at two target sites: CD34 and STAT1. DNA PK (M9831/VX-984) inhibitor was added to half the samples at a final concentration of 1 μM as an NHEJ inhibitor to demonstrate that NHEJ was occurring. SpOT-ON Cas9 and SpCas9 were compared. Cells were seeded and grown at described in Example 9. DNA was analyzed using deep targeted amplicon sequencing.

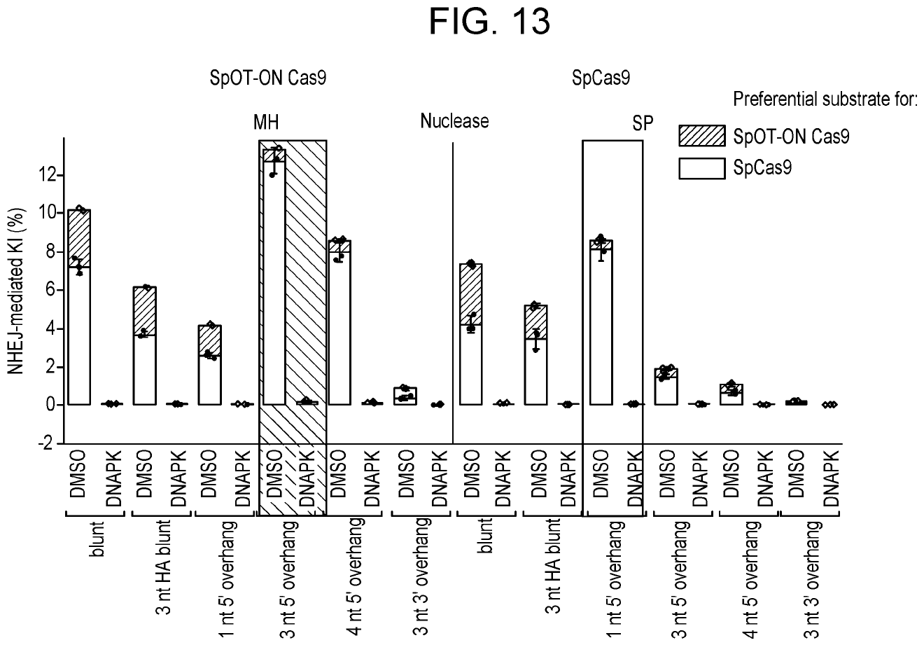

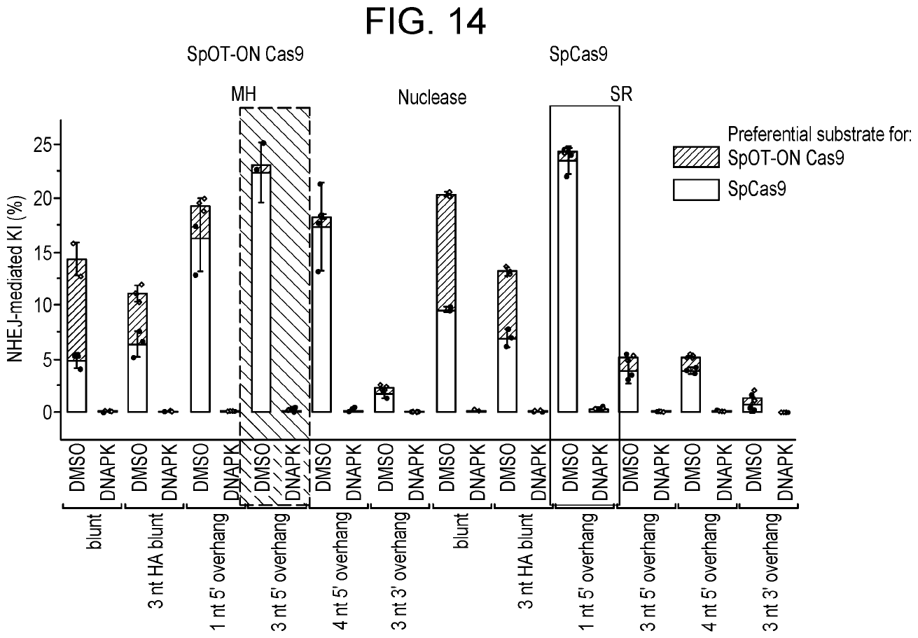

Results for knock-in at the CD34 locus are shown in FIG. 13. Results for knock-in at the STAT1 locus are shown in FIG. 14. As can be seen in FIG. 13 and FIG. 14, SpOT-ON shows best activity with its preferential substrate of a 3 nucleotide 5′ overhang (grey box), while SpCas9 shows best activity with it preferential substrate of a 1 nucleotide 5′ overhang (white box). Plots are shown for both potential directionalities of the insert, with dark grey representing forward (expected) insertion and light grey representing reverse insertion. As seen in the DNAPK columns, DNA-PK inhibitor treatment completely inhibits oligo donor insertion, proving that knock-in is NHEJ-mediated.

As is seen from the data, blunt ended dsDNA oligos are incorporated in the forward and reverse direction, after introducing a double-strand break with SpCas9 or SpOT-ON Cas9. Insertion via NHEJ is still seen when short homology arms of 3 nucleotides are introduced at both ends of the oligo.

SpOT-ON Cas9 enables targeted integration of dsDNA oligos with 5′ overhangs with 1 bp, 3 nucleotide and 4 nucleotide overhangs efficiently, with the highest efficiency for 3 nucleotide overhangs in a directional manner. SpCas9 shows high efficiency of dsDNA integration with 1 nucleotide overhangs, whereas dsDNA with 3 nucleotide or 4 nucleotide overhangs are integrated with low efficiency. These results suggest that SpOT-ON Cas9 DSB mainly leads to 3 nucleotide 5′ overhangs and SpCas9 shows staggered cuts with 1 nucleotide overhangs.

The efficiency of in cooperation of dsDNA oligos with 3′ overhangs (3 nucleotide) is lower than 5% for SpCas9 and SpOT-ON Cas9. This further supports the theory that of 5′ overhangs are generated.

### Example 12 Comparison of Different SpOT-ON Enzymes

Experiments were performed to improve the genome editing efficiency of SpOT-ON by introducing single amino acid substitutions at the residues in the vicinity of the DNA binding site. The SpOT-ON residues nearby the DNA binding site were defined by modelling the SpOT-ON structure with Alpha Fold2 (Jumper et al, Nature 2021) (see Table FIG. 16A). A subset of the residues for follow-up studies and mutagenesis was selected. For example, negative charges (aspartic acid (ASP or D) or glutamic acid (GLU or E)) were removed and/or positive charges (arginine (ARG or R) or lysine (LYS or K)) were introduced to increase the binding of the SpOT-ON complex to its target DNA. The SpOT-ON variants were generated by mutagenesis and transfected to cells as described in Example 5. Table 3 shows mutations that were tested for their impact on SpOT-ON activity. Overall, mutations in the vicinity of PAM-interacting regions (CTD domain) as well as in the REC3 domain of SpOT-ON show improved activity of the enzyme.

TABLE 4

| | Spot-ON | |
|---|---|---|
| Example | mutant | activity |
| 1 | R1336A | 0 |
| 2 | R1336K | 0 |
| 3 | R1389A | 0 |
| 4 | R1389K | 0 |
| 5 | R668A | + |
| 6 | N1164R | +++ |
| 7 | N1265R | ++++ |
| 8 | N1300R | ++++ |
| 9 | N1412R | ++++ |
| 10 | N347R | +++ |
| 11 | N348R | + |
| 12 | N562R | + |
| 13 | N565R | + |
| 14 | N618R | + |
| 15 | N651A | +++ |
| 16 | N651R | + |
| 17 | D1266R | ++++ |
| 18 | D309R | +++ |
| 19 | D345R | +++ |
| 20 | D487R | +++ |
| 21 | D607R | +++ |
| 22 | D30A | 0 |
| 23 | Q1129R | +++ |
| 24 | Q1381A | +++ |
| 25 | Q1381A | +++ |
| 26 | Q1381R | +++ |
| 27 | Q624R | + |
| 28 | Q661A | +++ |
| 29 | Q661R | + |
| 30 | Q713R | +++ |
| 31 | Q734R | +++ |
| 32 | E1032G | +++ |
| 33 | E1032R | +++ |
| 34 | E1409A | +++ |
| 35 | E1409R | + |
| 36 | E436R | ++++ |
| 37 | E610R | + |
| 38 | E611R | +++ |
| 39 | E691R | +++ |
| 40 | E697R | +++ |
| 41 | G1245R | + |
| 42 | G1335R | ++++ |
| 43 | H777A | + |
| 44 | 11242S | + |
| 45 | L125R | +++ |
| 46 | L125Y | + |
| 47 | L1162S | + |
| 48 | L1264S | +++ |
| 49 | L1299S | +++ |
| 50 | K1031R | +++ |
| 51 | K443R | + |
| 52 | K490R | +++ |
| 53 | K615R | +++ |
| 54 | K656R | +++ |
| 55 | F1035R | + |
| 56 | F620R | + |
| 57 | F636R | +++ |
| 58 | F670Y | + |
| 59 | S1243R | + |
| 60 | S1334A | +++ |
| 61 | S1334A | +++ |
| 62 | S1334R | ++++ |
| 63 | S1380R | ++++ |
| 64 | S1410R | +++ |
| 65 | S1413R | +++ |
| 66 | S634R | +++ |
| 67 | S638A | + |
| 68 | S638R | +++ |
| 69 | S711R | +++ |
| 70 | S1006R | +++ |
| 71 | S1017R | ++++ |
| 72 | T1267A | +++ |
| 73 | T1267R | ++++ |
| 74 | T1333A | + |
| 75 | T1333R | + |
| 76 | T551R | +++ |
| 77 | T639A | + |
| 78 | T639R | + |
| 79 | T640R | + |
| 80 | T666R | + |
| 81 | T897R | + |
| 82 | Y1338A | +++ |
| 83 | Y1338R | +++ |
| 84 | Y343R | + |
| 85 | Y566R | + |
| 86 | V1273S | +++ |
| 87 | V1274S | +++ |
| 88 | V486R | +++ |
| 89 | V644R | +++ |
| 90 | V660R | + |
| 91 | V660Y | + |
| 92 | V667R | + |
| 93 | V667S | + |
| 94 | V736R | ++++ |
| 95 | V736Y | ++++ |
| | WILD-TYPE | ++ |

What is claimed is:

1. A Cas9 effector protein comprising:
a) a first nuclear localization signal attached to the N-terminus of the Cas9 effector protein; and
b) a second nuclear localization signal attached to the C-terminus of the Cas9 effector protein.

2. The protein of claim 1, wherein the first nuclear localization signal is a monopartite nuclear localization signal.

3. The protein of claim 1, wherein the first nuclear localization signal is a bipartite nuclear localization signal.

4. The protein of any of claims 1 to 3, wherein the second nuclear localization signal is a monopartite nuclear localization signal.

5. The protein of any of claims 1 to 3, wherein the second nuclear localization signal is a bipartite nuclear localization signal.

6. The protein of claim 1, wherein the first nuclear localization signal is a bipartite nuclear localization signal and the second nuclear localization signal is a monopartite nuclear localization signal.

7. The protein of any of claims 2, 4 or 6, wherein the monopartite nuclear localization signal is SV40 Large T-Antigen, nucleoplasmin, EGL-13, c-Myc, TUS-protein nuclear localization signal, or combinations thereof.

8. The protein of any of claims 3, 5 or 6, wherein the bipartite nuclear localization signal is classical bipartite nuclear localization signal.

9. The protein of claim 6, wherein the first nuclear localization signal is classic bipartite nuclear localization signal and the second nuclear localization signal is SV40 Large T-Antigen nuclear localization signal.

10. The protein of any of claims 1 to 9, wherein the first nuclear localization signal is directly attached to the Cas9 effector protein.

11. The protein of any of claims 1 to 9, wherein the first nuclear localization signal is attached to the Cas9 effector protein via a linker.

12. The protein of any of claims 1 to 11, wherein the second nuclear localization signal is directly attached to the Cas9 effector protein.

13. The protein of any of claims 1 to 11, wherein the second nuclear localization signal is attached to the Cas9 effector protein via a linker.

14. The protein of claim 11 or 13, wherein the linker is a peptide linker having from 2 to 30 residues.

15. The protein of any of claims 1 to 14, wherein the protein comprises two copies of the first nuclear localization signal.

16. The protein of any of claims 1 to 14, wherein the protein comprises three copies of the first nuclear localization signal.

17. The protein of any of claims 1 to 14, wherein the protein comprises two copies of the second nuclear localization signal.

18. The protein of any of claims 1 to 14, wherein the protein comprises three copies of the second nuclear localization signal.

19. The protein of any of claims 1 to 18, wherein the Cas9 effector protein comprises a polypeptide sequence having at least 95% identity to any one of SEQ ID NOs: 10-97.

20. The protein of any of claims 1 to 18, wherein the Cas9 effector protein comprises a domain that matches a TIGR03031 protein family with an E-value cut-off of 1E-5.

21. The protein of any of claims 1 to 18, wherein the Cas9 effector protein comprises a polypeptide sequence at least 90% identical to SEQ ID NO: 98.

22. The protein of claim 21, wherein the polypeptide contains one or more modifications selected from N1164R, N1265R, N1300R, N1412R, N347R, N651A, D1266R, D309R, D345R, D487R, D607R, Q1129R, Q1381A, Q1381A, Q1381R, Q661A, Q713R, Q734R, E1032G, E1032R, E1409A, E436R, E611R, E691R, E697R, G1335R, L125R, L1264S, L1299S, K1031R, K490R, K615R, K656R, F636R, S1334A, S1334A, S1334R, S1380R, S1410R, S1413R, S634R, S638R, S711R, S1006R, S1017R, T1267A, T1267R, T551R, Y1338A, Y1338R, V1273S, V1274S, V486R, V644R, V736R and V736Y.

23. A CRISPR-Cas system comprising:
a) a Cas9 effector protein comprising:
i) a first nuclear localization signal attached to the N-terminus of the Cas9 effector protein; and
ii) a second nuclear localization signal attached to the C-terminus of the Cas9 effector protein; and
b) a guide polynucleotide comprising a guide sequence and forms a complex with the Cas9 effector protein, wherein the guide sequence is capable of hybridizing with a target sequence in a eukaryotic cell.

24. A CRISPR-Cas system comprising:
a) a nucleic acid sequence encoding a Cas9 effector protein comprising:
i) a first nuclear localization signal attached to the N-terminus of the Cas9 effector protein; and
ii) a second nuclear localization signal attached to the C-terminus of the Cas9 effector protein; and
b) a nucleic acid sequence encoding a guide polynucleotide that comprises a guide sequence and forms a complex with the Cas9 effector protein, wherein the guide sequence is capable of hybridizing with a target sequence in a eukaryotic cell.

25. The system of claim 24, wherein the nucleotide sequences of (a) and (b) are under control of a eukaryotic promoter.

26. The system of claim 24, wherein the nucleic acid sequences of (a) and (b) are in a single vector.

27. A CRISPR-Cas system comprising one or more vectors comprising:
a) a regulatory element operably linked to one or more nucleotide sequences encoding a Cas9 effector protein comprising:
i) a first nuclear localization signal attached to the N-terminus of the Cas9 effector protein; and
ii) a second nuclear localization signal attached to the C-terminus of the Cas9 effector protein; and
b) a guide polynucleotide that comprises a guide sequence and forms a complex with the Cas9 effector protein, wherein the guide sequence is capable of hybridizing with a target sequence in a eukaryotic cell.

28. The system of claim 27, wherein the regulatory element is a eukaryotic regulatory element.

29. The system of any of claims 23 to 28, wherein the first nuclear localization signal is a monopartite nuclear localization signal.

30. The system of any of claims 23 to 28, wherein the first nuclear localization signal is a bipartite nuclear localization signal.

31. The system of any of claims 23 to 30, wherein the second nuclear localization signal is a monopartite nuclear localization signal.

32. The system of any of claims 23 to 30, wherein the second nuclear localization signal is a bipartite nuclear localization signal.

33. The system of any of claims 23 to 28, wherein the first nuclear localization signal is a bipartite nuclear localization signal and the second nuclear localization signal is a monopartite nuclear localization signal.

34. The system of any of claims 23 to 28, wherein the first nuclear localization signal and the second nuclear localization signal are each bipartite nuclear localization signals.

35. The system of any of claims 29, 31 or 33, wherein the monopartite nuclear localization signal is SV40 Large T-Antigen, nucleoplasmin, EGL-13, c-Myc, TUS-protein nuclear localization signal, or combinations thereof.

36. The system of any of claims 30, 32 or 33, wherein the bipartite nuclear localization signal is classical bipartite nuclear localization signal.

37. The system of claim 33, wherein the first nuclear localization signal is classic bipartite nuclear localization signal and the second nuclear localization signal is SV40 Large T-Antigen nuclear localization signal.

38. The system of any of claims 23 to 37, wherein the first nuclear localization signal is directly attached to the Cas9 effector protein.

39. The system of any of claims 23 to 37, wherein the first nuclear localization signal is attached to the Cas9 effector protein via a linker.

40. The system of any of claims 23 to 39, wherein the second nuclear localization signal is directly attached to the Cas9 effector protein.

41. The system of any of claims 23 to 39, wherein the second nuclear localization signal is attached to the Cas9 effector protein via a linker.

42. The system of claim 39 or 41, wherein the linker is a peptide linker having from 2 to 30 residues.

43. The system of any of claims 23 to 42, wherein the protein comprises two copies of the first nuclear localization signal.

44. The system of any of claims 23 to 42, wherein the protein comprises three copies of the first nuclear localization signal.

45. The system of any of claims 23 to 44, wherein the protein comprises two copies of the second nuclear localization signal.

46. The system of any of claims 23 to 44, wherein the protein comprises three copies of the second nuclear localization signal.

47. The system of any of claims 23 to 46, wherein the Cas9 effector protein is derived from a bacterial species having a Type II-B CRISPR system.

48. The system of any of claims 23 to 46, wherein the Cas9 effector protein comprises a polypeptide sequence having at least 95% identity to any one of SEQ ID NOs: 10-97.

49. The system of any of claims 23 to 47, wherein the Cas9 effector protein comprises a domain that matches a TIGR03031 protein family with an E-value cut-off of 1E-5.

50. The system of any of claims 23 to 49, wherein the Cas9 effector protein comprises a polypeptide sequence at least 90% identical to SEQ ID NO: 98.

51. The system of any of claims 23 to 50, wherein the guide polynucleotide is an RNA.

52. The system of claim 51, wherein the guide sequence is from 19 to 30 bases in length.

53. The system of claim 51, wherein the guide sequence is from 19 to 25 bases in length.

54. The system of claim 51, wherein the guide sequence is from 21 to 26 bases in length.

55. The system of any of claims 23 to 54, wherein the guide polynucleotide further comprises a tracrRNA sequence.

56. The system of any of claims 23 to 55, wherein the Cas9 effector protein generates cohesive ends.

57. The system of any of claim 56, wherein the cohesive ends comprise a single-stranded polynucleotide overhang of 1 to 10 nucleotides.

58. The system of any of claim 56, wherein the cohesive ends comprise a single-stranded polynucleotide overhang of 2 to 6 nucleotides.

59. The system of any of claim 57, wherein the cohesive ends comprise a single-stranded polynucleotide overhang of 3 to 5 nucleotides.

60. A eukaryotic cell comprising the protein of any of claims 1 to 22.

61. A eukaryotic cell comprising the system of any of claims 23 to 59.

62. A delivery particle comprising the protein of any of claims 1 to 22.

63. A delivery particle comprising the system of any of claims 23 to 59.

64. The delivery particle of claim 63, wherein the Cas9 effector protein and the guide polynucleotide are in a complex.

65. The delivery particle of claim 64, wherein the complex further comprises a polynucleotide comprising a tracrRNA sequence.

66. The delivery particle of any of claims 62 to 65, further comprising a lipid, a sugar, a metal, or a protein.

67. A vesicle comprising the protein of any of claims 1 to 22.

68. A vesicle comprising the system of any of claims 23 to 59.

69. The vesicle of claim 68, wherein the Cas9 effector protein and the guide polynucleotide are in a complex.

70. The vesicle of claim 68, further comprising a polynucleotide comprising a tracrRNA sequence.

71. The vesicle of any of claims 67 to 70, wherein the vesicle is an exosome or a liposome.

72. A viral vector comprising the protein of any of claims 1 to 22.

73. A viral vector comprising the system of any of claims 23 to 59.

74. The viral vector of claim 73, further comprising a nucleic acid sequence encoding a tracrRNA sequence.

75. The viral vector of any of claims 72 to 74, wherein the viral vector is an adenovirus particle, an adeno-associated virus particle or a herpes simplex virus particle.

76. A method for providing site-specific modification of a target sequence in a eukaryotic cell, the method comprising:
a) introducing into the cell:
i) a nucleotide encoding a Cas9 effector protein comprising:
A) a first nuclear localization signal attached to the N-terminus of the Cas9 effector protein; and
B) a second nuclear localization signal attached to the C-terminus of the Cas9 effector protein; and
ii) a nucleotide encoding a guide polynucleotide that forms a complex with the Cas9 effector protein and comprises a guide sequence, wherein the guide sequence is capable of hybridizing with a target sequence in host polynucleotide;
b) generating cohesive ends in the host polynucleotide with the Cas9 effector protein and the guide polynucleotide; and
c) ligating
i) the cohesive ends of (b) together, or
ii) a 3′ end of a polynucleotide sequence of interest to one cohesive end, and a 5′ end of the polynucleotide sequence of interest to one cohesive end; thereby modifying the target sequence.

77. The method of claim 76, wherein the first nuclear localization signal is a monopartite nuclear localization signal.

78. The method of claim 76, wherein the first nuclear localization signal is a bipartite nuclear localization signal.

79. The method of any of claims 76 to 78, wherein the second nuclear localization signal is a monopartite nuclear localization signal.

80. The method of any of claims 76 to 78, wherein the second nuclear localization signal is a bipartite nuclear localization signal.

81. The method of any of claims 76 to 80, wherein the first nuclear localization signal is a bipartite nuclear localization signal and the second nuclear localization signal is a monopartite nuclear localization signal.

82. The method of any of claims 77, 79 or 81, wherein the monopartite nuclear localization signal is SV40 Large T-Antigen, nucleoplasmin, EGL-13, c-Myc, TUS-protein nuclear localization signal, or combinations thereof.

83. The method of any of claims 78, 80 or 81, wherein the bipartite nuclear localization signal is classical bipartite nuclear localization signal.

84. The method of any of claims 76 to 83, wherein the first nuclear localization signal and the second nuclear localization signal are each a bipartite nuclear localization signal.

85. The method of claim 81, wherein the first nuclear localization signal is classic bipartite nuclear localization signal and the second nuclear localization signal is SV40 Large T-Antigen nuclear localization signal.

86. The method of any of claims 76 to 85, wherein the first nuclear localization signal is directly attached to the Cas9 effector protein.

87. The method of any of claims 76 to 85, wherein the first nuclear localization signal is attached to the Cas9 effector protein via a linker.

88. The method of any of claims 76 to 87, wherein the second nuclear localization signal is directly attached to the Cas9 effector protein.

89. The method of any of claims 76 to 87, wherein the second nuclear localization signal is attached to the Cas9 effector protein via a linker.

90. The method of claim 87 or 89, wherein the linker is a peptide linker having from 2 to 30 residues.

91. The method of any of claims 76 to 90, wherein the protein comprises two copies of the first nuclear localization signal.

92. The method of any of claims 76 to 90, wherein the protein comprises three copies of the first nuclear localization signal.

93. The method of any of claims 76 to 92, wherein the protein comprises two copies of the second nuclear localization signal.

94. The method of any of claims 76 to 92, wherein the protein comprises three copies of the second nuclear localization signal.

95. The method of any of claims 76 to 94, wherein the Cas9 effector protein is derived from a bacterial species having a Type II-B CRISPR system.

96. The method of any of claims 76 to 95, wherein the Cas9 effector protein comprises a polypeptide sequence having at least 95% identity to any one of SEQ ID NOs: 10-97.

97. The method of any of claims 76 to 95, wherein the Cas9 effector protein comprises a domain that matches a TIGR03031 protein family with an E-value cut-off of 1E-5.

98. The method of any of claims 76 to 97, wherein the guide polynucleotide is an RNA.

99. The method of claim 98, wherein the guide polynucleotide is from 19 to 30 bases in length.

100. The method of claim 98, wherein the guide polynucleotide is from 19 to 25 bases in length.

101. The method of claim 98, wherein the guide polynucleotide is from 21 to 26 bases in length.

102. The method of any of claims 76 to 101, wherein the guide polynucleotide further comprises a tracrRNA sequence.

103. The system of any of claims 76 to 102, wherein the Cas9 effector protein generates cohesive ends.

104. The method of any of claims 76 to 103, wherein the cohesive ends comprise a single-stranded polynucleotide overhang of 1 to 10 nucleotides.

105. The method of any of claims 76 to 104, wherein the cohesive ends comprise a single-stranded polynucleotide overhang of 2 to 6 nucleotides.

106. The method of any of claims 76 to 105, wherein the cohesive ends comprise a single-stranded polynucleotide overhang of 3 to 5 nucleotides.

107. The method of any of claims 76 to 106, wherein the cohesive ends are blunt ends.

108. The method of any of claims 76 to 106, wherein the cohesive ends have a 5′ single-stranded polynucleotide overhang.

109. The method of any of claims 76 to 106, wherein the cohesive ends have a 3′ single-stranded polynucleotide overhang.

110. The method of any one of claims 76 to 109, wherein the eukaryotic cell is an animal or human cell.

111. The method of any one of claims 76 to 109, wherein the eukaryotic cell is a human cell.

112. The method of any one of claims 76 to 109, wherein the eukaryotic cell is a plant cell.

113. The method of any one of claims 76 to 112, wherein the modification is deletion of at least part of the target sequence.

114. The method of any one of claims 76 to 112, wherein the modification is mutation of the target sequence.

115. The method of any one of claims 76 to 112, wherein the modification is inserting a sequence of interest into the target sequence.

116. A method for reducing degradation of Cas9 effector protein in a cell comprising
a) attaching a first nuclear localization signal to the N-terminus of the Cas9 effector protein; and
b) attaching a second nuclear localization signal to the C-terminus of the Cas9 effector protein.

117. The method of claim 116, wherein the first nuclear localization signal is a monopartite nuclear localization signal.

118. The method of claim 116, wherein the first nuclear localization signal is a bipartite nuclear localization signal.

119. The method of any of claims 116 to 118, wherein the second nuclear localization signal is a monopartite nuclear localization signal.

120. The method of any of claims 116 to 118, wherein the second nuclear localization signal is a bipartite nuclear localization signal.

121. The method of claim 116, wherein the first nuclear localization signal is a bipartite nuclear localization signal and the second nuclear localization signal is a monopartite nuclear localization signal.

122. The method of any of claims 117, 119, or 121, wherein the monopartite nuclear localization signal is SV40 Large T-Antigen, nucleoplasmin, EGL-13, c-Myc, TUS-protein nuclear localization signal, or combinations thereof.

123. The method of any of claims 118, 120, or 121, wherein the bipartite nuclear localization signal is classical bipartite nuclear localization signal.

124. The method of claim 121, wherein the first nuclear localization signal is classic bipartite nuclear localization signal and the second nuclear localization signal is SV40 Large T-Antigen nuclear localization signal.
